(19) United States
(12) Patent Application Publication
(10) Pub. No.: US 2005/0164324 A1
(43) Pub. Date: Jul. 28, 2005

(54) Systems, methods and kits for characterizing phosphoproteomes

(76) Inventor: Steven P. Gygi, Foxboro, MA (US)

Correspondence Address: George W. Neuner;Edwards & Angell, LLP, P. O. Box 55874, Boston, MA, 02205, US

(21) Appl. No.: 10/862,195

(22) Filed: Jun. 4, 2004

Related U.S. Application Data
(60) Provisional application No. 60/476,010, filed on Jun. 4, 2003.

(51) Int. Cl. C12Q 1/37; G01N 33/00

(52) U.S. Cl. 435/23; 436/86

(57) ABSTRACT

The invention provides systems, software, methods and kits for detecting and/or quantifying phosphorylatable polypeptides and/or acetylated polypeptides in complex mixtures, such as a lysate of a cell or cellular compartment (e.g., such as an organelle). The methods can be used in high throughput assays to profile phosphoproteomes and to correlate sites and amounts of phosphorylation with particular cell states.

### CROSS-REFERENCE TO RELATED APPLICATIONS

This application claims priority from U.S. Ser. No. 60/476,010 filed Jun. 4, 2003.

### GOVERNMENT GRANTS

This work was supported by NIH grants 5K22HG000041 and GM67945. The government may have certain rights in this invention.

### FIELD OF THE INVENTION

This invention provides methods, systems, software and kits for characterizing phosphoproteomes. In particular, the invention provides methods, systems, software and kits for identifying differential protein phosphorylation, for quantifying phosphorylated proteins and for identifying modulators of phosphorylated proteins.

### BACKGROUND OF THE INVENTION

Determining the site of a regulatory phosphorylation event can often unlock the specific biology surrounding a disease, elucidate kinase-substrate relationships, and provide a handle to study the regulation of an essential pathway. Although the events leading up to and directly following protein phosphorylation are the subject of intense research efforts, the large-scale identification and characterization of phosphorylation sites is an unsolved problem.

Methods for evaluating gene expression patterns that capture data relating to the abundance of proteins in a cell typically fail to provide information regarding post-translational modifications of proteins. Such information may be critical in determining the activity of expressed proteins. For example, many proteins are initially translated in an inactive form and upon modification, gain biological function. The addition of biochemical groups to translated polypeptides has effects on protein stability, oligomerization, protein secondary/tertiary structure, enzyme activity and more globally on signaling pathways in cells.

The activity of numerous proteins and association of proteins into functional complexes are frequently controlled by reversible protein phosphorylation (see, e.g., Graves, et al., Pharmacol. Ther. 82, 111-121, 1999; Koch, et al., Science 252, 668-674, 1991; Hunter, Semin. Cell Biol. 5, 367-376, 1994). Phosphorylation occurs by the addition of phosphate to polypeptides by specific enzymes known as protein kinases. Phosphate groups are added to, for example, tyrosine, serine, threonine, histidine, and/or lysine amino acid residues depending on the specificity of the kinase acting upon the target protein.

Reversible protein phosphorylation is a general event affecting countless cellular processes. The identification of phosphorylation sites is most commonly accomplished by mass spectrometry. Tandem mass spectrometry provides the ability to fragment the phosphopeptide to determine its sequence as well as pinpoint the specific serine, threonine, or tyrosine modified by a protein kinase. While protein sequence analysis by mass spectrometry is a mature technology with many papers reporting protein identifications in the thousands, the large-scale determination of phosphorylation sites is only just emerging. In fact, the two largest repositories of determined sites were both from yeast studies with 383 and 125 sites detected, respectively. Ficarro, S. B. et al., Nat Biotechnol 20, 301-5. (2002); Peng, J. et al., Nat Biotechnol 21, 921-6 (2003). In human cells, 64 sites were determined from a single sample. Ficarro, S. et al., J Biol Chem 278, 11579-89 (2003).

To date several disease states have been linked to the abnormal phosphorylation/dephosphorylation of specific proteins. For example, the polymerization of phosphorylated tau protein allows for the formation of paired helical filaments that are characteristic of Alzheimer's disease, and the hyperphosphorylation of retinoblastoma protein (pRB) has been reported to progress various tumors (see, e.g., Vanmechelen et al. Neurosci. Lett. 285:49-52, 2000, and Nakayama et al. Leuk. Res. 24:299-305, 2000).

The identification of phosphorylation sites on a protein is complicated by the facts that proteins are often only partially phosphorylated and that they are often present only at very low levels. Prior art methods for identifying phosphorylated proteins have included in vivo incorporation of radiolabeled phosphate and analysis of labeled proteins by electrophoresis and autoradiography, western blotting using antibodies specific for phosphorylated forms of target proteins, and the use of yeast systems to identify mutations in protein kinases and/or protein phosphatases. Generally, only highly expressed proteins are detectable using these techniques and it is difficult to readily identify the sequences of the modified proteins. Immunological methods can only detect phosphorylated proteins globally (e.g., an anti-phosphotyrosine antibody will detect all tyrosine-phosphorylated proteins).

The development of methods and instrumentation for mass spectrometry has significantly increased the sensitivity and speed of the identification of phosphorylated proteins. Several mass spectrometry based techniques have been employed for the mapping of phosphorylation sites. For example, Cao, et al, Rapid Commun. Mass Spectrom. 14: 1600-1606, 2000, report mapping phosphorylation sites of proteins using on-line immobilized metal affinity chromatography (IMAC)/capillary electrophoresis (CE)/electrospray ionization multiple stage tandem mass spectrometry (MS). The IMAC resin retains and preconcentrates phosphorylated proteins and peptides; CE separates the phosphopeptides of a mixture eluted from the IMAC resin, and MS provides information including the phosphorylation sites of each component.

Posewitz, et al., Anal. Chem. 71:2883-2892, 1999, reports using immobilized metal affinity chromatography in a microtip format to isolate phosphopeptides for direct analysis by matrix-assisted laser desorption/ionization time of flight and nanoelectrospray ionization mass spectrometry.

Enrichment analysis of phosphorylated proteins also has been used to probe the phosphoproteome (Chait et al., Nature Biotechnology 19: 379-382, 2001).

However, there are two major obstacles to phosphorylation site analysis, regardless of scale of the experiment. First, fragmentation of phosphopeptides by collision-induced dissociation in a tandem mass spectrometer commonly results in the production of a single dominant peak corresponding to a neutral loss of phosphoric acid (H₃PO₄, 98 daltons) from the phosphopeptide. The lack of informative fragmentation at the peptide backbone severely reduces the precision of database searching algorithms to identify the phosphopeptide. In addition, when a phosphopeptide is identified, it is often not possible to define the site to a particular serine, threonine, or tyrosine residue due to the lack of informative fragmentation².

Another major obstacle to phosphorylation analysis is the often poor stoichiometry of the phosphorylated protein compared to the nonphosphorylated protein compounded by the already low expression levels of most phosphoproteins. For this reason, phosphopeptides are not readily detected from the direct analysis of complex proteolyzed protein mixtures even when multidimensional chromatography is used. It is essential to employ some type of enrichment strategy to overcome the tremendous complexity that a proteolyzed lysate represents. Efforts to isolate phosphopeptides in the past have utilized either i) chemical modification of phosphate groups, ii) phosphate-specific mass spectrometry-based methods, or iii) affinity-based methods (antibody or metal ion chromatography). Regardless of the enrichment procedure, amino acid sequence analysis and site determination were accomplished by tandem mass spectrometry. Each technique has been successful for the analysis of a few proteins (<30), but only IMAC has shown the potential for the identification of more than a few sites from complex mixtures.

Thus, new and better methods for analysis of proteins and determining the site of a regulatory phosphorylation event continue to be sought.

### SUMMARY OF THE INVENTION

The ability to quickly screen for alterations in the phosphorylation state of proteins is important to characterize intra and inter cellular signaling events required for normal physiological responses. Identification and/or quantification of phosphorylatable proteins facilitates development of improved diagnostics for the detection of various disease states as well as providing candidate drug targets for developing treatment regimens.

The invention provides methods for screening for phosphorylatable polypeptides (e.g., including proteins and peptides) to determine sites of phosphorylation, numbers of phosphates present in a phosphorylated polypeptide, and/or the level of a phosphorylated or unphosphorylated form of a phosphorylatable polypeptide in a sample.

In one aspect, the method comprises separating a plurality of proteins according to at least one biological property, e.g., such as molecular weight, obtaining subsets of separated polypeptides, contacting the subsets with a protease activity to obtain peptides corresponding to each subset of separated polypeptides, and enriching for peptides comprising positive charges (e.g., from 1+ to 4+). Preferably, the enriched fraction so obtained is enriched for phosphorylated peptides.

In another aspect, the method comprises the identification of the N-terminal peptide of proteins after trypsin digestion. The trypsin digestion provides an acetylated N terminus of a peptide with a solution charge state of 1+ at pH 3.

In one aspect, separation according to the at least one biological property comprises separation according to molecular weight, such as by gel electrophoresis and subsets are obtained by cut a gel comprising electrophoresed proteins into sections and evaluating peptide digests of separated polypeptides within each gel section. In another aspect, separation according to the at least one biological property is based on binding affinity to a binding partner (e.g., such as by chromatography on an IMAC column). Separation also may be based on hydrophobicity, hydrophilicity, the presence of particular sequence domains and the like. However, in one aspect, separation of polypeptides is performed randomly, merely to reduce the complexity of the sample of polypeptides prior to further analysis.

In one particularly preferred aspect, enrichment is achieved by separating the peptides in each subset according to charge using strong cation exchange chromatography (SCX) at a pH of about 3 and selecting initial fractions eluted from the column. Preferably, data-dependent acquisition of MS³ spectra for improved phosphopeptide identification also is utilized.

Phosphorylation sites within the phosphorylated peptides can be identified using methods known in the art or described herein. In one aspect, such a method comprises obtaining a peptide to be analyzed, generating a first series of precursor ions corresponding to the peptide, and a second series of fragment ions obtained by fragmentation of selected precursor ions, and, detecting, among the fragment ions, a fragment ion having the signature predicted for a modified amino acid. In another aspect, the mass of a fragment ion is compared to the mass of a reference ion characteristic of a phosphorylated amino acid, thereby identifying the phosphorylation state of the peptide being analyzed. As the initial fractions provide greater than 100,000 different peptides, expression profiles of modified peptides can be determined rapidly and efficiently for proteomes of cells and cell compartments.

In a further aspect, the invention provides a method for comparing the phosphorylation state of one or more proteins in a plurality of samples and for identifying and/or individually quantitating phosphorylated proteins.

The invention also provides a method for generating a peptide internal standard for detecting and quantifying phosphorylated proteins. The method comprises identifying a peptide digestion product of a target polypeptide comprising at least one phosphorylation site, determining the amino acid sequence of a peptide digestion product comprising a phosphorylation site and synthesizing a peptide having the amino acid sequence. The peptide is labeled with a mass-altering label (e.g., by incorporating labeled amino acid residues during the synthesis process) and fragmented (e.g., by multi-stage mass spectrometry). Preferably, the label is a stable isotope. A peptide signature diagnostic of the peptide is determined, after one or more rounds of fragmenting, and the signature is used to identify the presence and/or quantity of a peptide of identical amino acid sequence in a sample and to detect the presence or absence of the modification. In one aspect, panels of peptide internal standards are generated corresponding to (i.e., diagnostic of) different modified forms of the same protein (i.e., proteins which are phosphorylated at more than one site and/or which comprise other types of modifications (e.g., glycosylation, ubiquitination, acetylation, farnesylation, and the like).

Peptide internal standards corresponding to different peptide subsequences of a single target protein also can be generated to provide for redundant controls in a quantitative assay. In one aspect, different peptide internal standards corresponding to the same target protein are generated and differentially labeled (e.g., peptides are labeled at multiple sites to vary the amount of heavy label associated with a given peptide).

In a further aspect, a panel of peptide internal standards corresponding to amino acid subsequences of at least one phosphorylatable protein in a molecular pathway is generated. Preferably, internal standards corresponding to a plurality of phosphorylatable peptides are generated. In one aspect, the panel further comprises peptide internal standard(s) corresponding to one or more protein kinases or phosphatases.

Molecular pathways, include, but are not limited to signal transduction pathways, cell cycle pathways, metabolic pathways, blood clotting pathways, and the like. In one aspect, the panel includes peptide standards which correspond to different phosphorylated forms of one or more proteins in a pathway and the panel is used to determine the presence and/or quantity of the activated or inactivated form of a pathway protein.

In a further aspect, the invention provides a method for identifying a treatment that modulates phosphorylation of an amino acid in a target polypeptide, comprising: subjecting a sample containing the target polypeptide to a treatment, determining the level of phosphorlyation of one or more amino acids in the target polypeptide, both before and after the treatment; identifying a treatment that results in a change of the level of modification of the one or more amino acids after the treatment. The treatment may comprise exposure to an agent (e.g., such as a drug) or exposure to a condition (e.g., such as pH, temperature, etc.)

In one aspect, a labeled peptide internal standard and target peptide (i.e., a peptide being detected in a sample) are fragmented (e.g., using multistage mass spectrometry) and the ratio of labeled fragments to unlabeled fragments; is determined. The quantity of the target polypeptide can be calculated using both the ratio and known quantity of the labeled internal standard. The mixtures of different polypeptides can include, but are not limited to, such complex mixtures as a crude fermenter solution, a cell-free culture fluid, a cell or tissue extract, blood sample, a plasma sample, a lymph sample, a cell or tissue lysate; a mixture comprising at least about 100 different polypeptides; at least about 1000 different polypeptides, at least about 100, 000 different polypeptides. or a mixture comprising substantially the entire complement of proteins in a cell or tissue. In one preferred aspect, the method is used to determine the presence of and/or quantity of one or more target polypeptides directly from one or more cell lysates, i.e., without separating proteins from other cellular components or eliminating other cellular components.

In a still further aspect of the invention, stable isotope labeling with amino acids in cell culture, or SILAC, is used. Cells representing two biological conditions are cultured in amino acid-deficient growth media supplemented with ¹²C- or ¹³C-labeled amino acids, e.g., Arg or Lys. The proteins in these two cell populations effectively become isotopically labeled as “light” or “heavy.” The cells are isolated, mixed in equal ratios and processed. the method further includes co-eluting the proteins by chromatographic separation into the mass spectrometer, gathering relative quantitative information for each protein by calculating the ratio of intensities of the two peaks produced in the peptide mass spectrum (MS scan), and acquiring sequence data for these peptides by fragment analysis in the product ion mass spectrum (MS/MS scan), thereby providing accurate protein identification.

In one aspect, the presence and/or quantity of target polypeptide in a mixture are diagnostic of a cell state. In another aspect, the cell state is representative of an abnormal physiological response, for example, a physiological response which is diagnostic of a disease. In a further aspect, the cell state is a state of differentiation or represents a cell which has been exposed to a condition or agent (e.g., a drug, a therapeutic agent, a potential toxin). In one aspect, the method is used to diagnose the presence or risk of a disease. In another aspect, the method is used to identify a condition or agent which produces a selected cell state (e.g., to identify an agent which returns one or more diagnostic parameters of a cell state to normal).

In a further aspect, the method comprises determining the presence and/or quantity of target peptides in at least two mixtures. In another aspect, one mixture is from a cell having a first cell state and the second mixture is from a cell having a second cell state. In a further aspect, the first cell is a normal cell and the second cell is from a patient with a disease. In still a further aspect, the first cell is exposed to a condition and/or treated with an agent and the second cell is not exposed and/or treated. Preferably, first and second mixtures are evaluated in parallel. The methods can be used to identify regulators of phosphorylation, e.g., such as kinases and phosphatases. The agent may be a therapeutic agent for treating a disease associated with an improper state of phosphorylation (e.g., abnormal sites or amounts of phosphorylation). Suitable agents include, but are not limited to, drugs, polypeptides, peptides, antibodies, nucleic acids (genes, cDNAs, RNA's, antisense molecules, ribozymes, aptamers and the like), toxins, and combinations thereof.

Alternatively, the two mixtures can be from identical samples or cells. In one aspect, a labeled peptide internal standard is provided in different known amounts in each mixture. In another aspect, pairs of labeled peptide internal standards are provided each comprising mass-altering labels which differ in mass, e.g., by including different amounts of a heavy isotope in each peptide.

The invention also provides a method of determining the presence of and/or quantity of a phosphorylation in a target polypeptide. Preferably, the label in the internal standard is part of a peptide comprising a modified amino acid residue or to an amino acid residue which is predicted to be modified in a target polypeptide. In one aspect, the presence of the modification reflects the activity of a target polypeptide and the assay is used to detect the presence and/or quantity of an active polypeptide. The method is advantageous in enabling detection of small quantities of polypeptide (e.g., about 1 part per million (ppm) or less than about 0.001% of total cellular protein).

The presence and/or quantity of phosphorylated proteins can be used to profile the function of a pathway in a particular cell. In one aspect, the pathway is one or more of a signal transduction pathway, a cell cycle pathway, a metabolic pathway, a blood clotting pathway and the like. The coordinate function of multiple pathways can be evaluated using a plurality of panels of standards.

The invention further provides reagents useful for performing the method described above. In one aspect, a reagent according to the invention comprises a peptide internal standard comprising a phosphorylation site labeled with a stable isotope. Preferably, the standard has a unique peptide fragmentation signature diagnostic of the phosphorylation state of the peptide. In one aspect, the peptide is phosphorylated. In another aspect, the peptide is unphosphorylated. In a further aspect, a pair of peptides is provided, a peptide internal standard corresponding to a phosphorylated peptide and a peptide internal standard corresponding to a peptide identical in sequence but not phosphorylated. In another aspect, the peptide is a subsequence of a known protein and can be used to identify the presence of and/or quantify the protein in sample, such as a cell lysate. In one aspect, the peptide internal standard comprises a label associated with a modified amino acid residue, such as a phosphorylated amino acid residue, a glycosylated amino acid residue, an acetylated amino acid residue, a famesylated residue, a ribosylated residue, and the like.

In another aspect, panels of peptide internal standards corresponding to different amino acid subsequences of single polypeptide are provided, including peptides comprising phosphorylation sites and peptides lacking phosphorylation sites.

In a further aspect, panels of peptide internal standards are provided which correspond to different proteins in a molecular pathway (e.g., a signal transduction pathway, a cell cycle pathway, a metabolic pathway, a blood clotting pathway and the like). In still a further aspect, peptide internal standards corresponding to different modified forms of one or more proteins in a pathway are provided.

In still a further aspect, panels of peptide internal standards are provided which correspond to proteins diagnostic of different diseases, allowing a mixture of peptide internal standards to be used to test for the presence of multiple diseases in a single assay.

The invention additionally provides kits comprising one or more peptide internal standards labeled with a stable isotope. In one aspect, a kit comprises peptide internal standards comprising different peptide subsequences from a single known protein. In another aspect, the kit comprises peptide internal standards corresponding to different variant forms of the same amino acid subsequence of a target polypeptide. In still another aspect, the kit comprises peptide internal standards corresponding to different known or predicted modified f6rms of a polypeptide. In a further aspect, the kit comprises peptide internal standards corresponding to sets of related proteins, e.g., such as proteins involved in a molecular pathway (a signal transduction pathway, a cell cycle, etc) and/or to different modified forms of proteins in the pathway. In still a further aspect, a kit comprises a labeled peptide internal standard as described above and software for performing multistage mass spectrometry.

The kit may also include a means for obtaining access to a database comprising data files which include data relating to the mass spectra of fragmented peptide ions generated from peptide internal standards. The means for obtaining access can be provided in the form of a URL and/or identification number for accessing a database or in the form of a computer program product comprising the data files. In one aspect, the kit comprises a computer program product which is capable of instructing a processor to perform any of the methods described above.

The present invention also provides a system and software for facilitating the analysis of phosphoproteomes. The invention provides a system that comprises a relational database which stores mass spectral data relating to phoshorylation states for a plurality of proteins in a proteome. The system further comprises a data analysis system for correlating phosphorylation states to one or more characteristics relating to the source of the proteome, e.g., a cell or tissue extract, a patient group, etc.

Such characteristics include, but are not limited to: the activity of a kinase in the cell or tissue extract, the activity of a phosphatase in the cell or tissue extract, presence/absence of a disease in the source of the sample (i.e., a patient from whom the sample is obtained); stage of a disease; risk for a disease; likelihood of recurrence of disease; a shared genotype at one or more genetic loci; exposure to an agent (e.g., such as a toxic substance or a potentially toxic substance, a carcinogen, a teratogen, an environmental pollutant, a therapeutic agent such as a candidate drug, a nucleic acid, protein, peptide, small molecule, etc.) or condition (temperature, pH, etc); a demographic characteristic (age, gender, weight; family history; history of preexisting conditions, etc.); resistance to agent, sensitivity to an agent (e.g., responsiveness to a drug) and the like.

In one aspect, the data management program comprises a data analysis program for identifying similarities of features of mass spectral signatures for one or more peptides in a plurality of peptides with mass spectral signatures for known peptides. In another aspect, the data analysis program identifies the amino acid sequences for one or more peptides in the plurality of peptides. In still another aspect, the plurality of peptides is a mixture of labeled peptides, a first set of peptides labeled with a first label and a second set of peptides labeled with a second label. In a further aspect, the first label has a first mass and the second label has a second, different mass. Preferably, the data analysis system comprises a component for determining the relative abundance of a first labeled peptide with a second labeled peptide.

In one aspect, the system is connectable to one or more external databases through a network server, such databases comprising genomic, proteomic, pharmacological data and the like.

The invention also provides a method for storing peptide data to a database. The method comprises acquiring mass spectrum signatures for one or more peptides in a plurality of peptides. The one or more peptides exist in a phosphorylated form in one or more cells having a cell state (e.g., a differentiation state, an association with a disease or response to an abnormal physiological condition, response to an agent, and the like). The signatures are stored in a database and correlated with the presence or absence of cell state. Preferably, pairs of signatures associated with both the phosphorylated and unphosphorylated states of the peptides are stored in the database. In one aspect, the mass spectrum signatures are obtained using mass analytical techniques, including, but not limited to: multistage mass spectroscopy, electron ionization mass analysis, fast atom/ion bombardment mass analysis, matrix-assisted laser desorption/ionization mass analysis and electrospray ionization mass analysis, and the like

Preferaby, mass spectral data is obtained by separating a peptide mixture according to mass and charge characteristics and subjecting separated peptides to one or more mass analyses where each peptide is fragmented and additional mass spectral signatures corresponding to fragmented peptides are produced.

The amino acid sequences of the peptides are determined using methods known in the art. See, e.g., U.S. Pat. No. 6,017,693 and U.S. Pat. No. 5,538,897. In one aspect, mass spectra from an experiment are input into a computer containing a database of sequence-associated spectrum. The computer then performs a search of the database and outputs results. Preferably, mass spectra are automatically queried against a database of spectral information to generate sequence information.

Differentially expressed phosphorylated peptides are correlated by the system with responses of a proteome to a stimulus, a condition, an agent (e.g., a therapeutic agent such as a drug, a toxic agent or potentially toxic agent, a carcinogen or potential carcinogen), a change in environment (e.g., nutrient level, temperature, passage of time), a disease state, malignancy, site-directed mutation, introduction of exogenous molecules (nucleic acids, polypeptides, small molecules, etc.) into a cell, tissue or organism from which the sample originated and other characteristics as described above.

### BRIEF DESCRIPTION OF THE FIGURES

The objects and features of the invention can be better understood with reference to the following detailed description and accompanying drawings.

FIGS. 1A-C illustrate a method according to one aspect of the invention and illustrates how strong cation exchange chromatography separates peptides by solution charge. FIG. 1A shows the separation of a complex peptide mixture by SCX chromatography with fraction collection every minute. Each fraction was analyzed by microcapillary LC-MS/MS techniques. FIG. 1B shows the number of unique peptides identified in each fraction by the Sequest algorithm for each solution charge state. FIG. 1C shows a mixed mode separation of polysulfoethyl-aspartamide based primarily on ionic charge but also on hydrophobicity.

FIG. 2 shows a flowchart for large-scale analysis of nuclear protein. A nuclear preparation from HeLa cells (10 mg) was separated on a single SDS-PAGE preparative gel. Twenty regions (slices) were removed from the gel and subjected to in-gel tryptic digestion. The 20 complex peptide samples were separated further by strong cation exchange (SCX) chromatography with fraction collection every minute. Each fraction (n=1000) was then subjected to analysis by nano-scale microcapillary LC-MS/MS.

FIG. 3 shows SCX chromatography separation of Slice 14 with respect to number of unique peptides identified per fraction. Upper panel shows the separation with UV detection at 214 nm. Fractions (200 microliters) were collected every minute. Each fraction was analyzed by LC-MS/MS with a 2-hr gradient. Peptides in each fraction were identified by Sequest (REF). Peptides identified having different solution charge states are shown in the lower panel.

FIG. 4A shows mass spectral data for and the amino acid sequence of a peptide obtained using a method according to the invention. The peptide is a subsequence of the human polypeptide KP58_HUMAN. FIG. 4B shows mass spectral data for and the amino acid sequence of a peptide obtained using a method according to the invention. The peptide is a subsequence of the polypeptide GP:AB033054. FIG. 4C shows mass spectral data for and the amino acid sequence of a peptide obtained using a method according to the invention. The peptide is a subsequence of the polypeptide WEE1_HUMAN. FIG. 4D shows mass spectral data for and the amino acid sequence of a peptide obtained using a method according to the invention. The peptide is a subsequence of the polypeptide PIR2:A38282. FIG. 4E shows mass spectral data for and the amino acid sequence of a peptide obtained using a method according to the invention. The peptide is a subsequence of the polypeptide PYRG_HUMAN. FIG. 4F shows mass spectral data for and the amino acid sequence of a peptide obtained using a method according to the invention. The peptide is a subsequence of the polypeptide GP:Y18004. FIG. 4G shows mass spectral data for and the amino acid sequence of a peptide obtained using a method according to the invention. The peptide is a subsequence of the polypeptide GP:AF161470. FIG. 4H shows mass spectral data for and the amino acid sequence of a peptide obtained using a method according to the invention. The peptide is a subsequence of the polypeptide S3B2_HUMAN. FIG. 4I shows mass spectral data for and the amino acid sequence of a peptide obtained using a method according to the invention. The peptide is a subsequence of the polypeptide GB:BC01 1630.

FIG. 5A shows neutral loss of each fraction obtained by SCX from slice 14 as described in Example 1. FIG. 5B shows control random loss of fractions, i.e., reflecting the level of variability or background in the analysis. FIG. 5C shows numbers of neutral losses (y-axis) vs. fraction number.

FIGS. 6A-C shows a scheme for phosphopeptide enrichment by strong cation exchange (SCX) chromatography. FIG. 6A shows, At pH 2.7, peptides produced by trypsin proteolysis generally have a solution charge state of 2⁺ while phosphopeptides have a charge state of only 1⁺. FIG. 6B shows solution charge state distribution of peptides (5-40 amino acids in length) produced by a theoretical digestion of the human protein database with trypsin (n=6.8×10⁸ peptides). Sixty-eight percent of the predicted peptides have a net charge of 2⁺. Any peptide in this category would shift to a 1⁺ charge state upon phosphorylation. FIG. 6C shows SCX chromatography separation at pH 2.7 for a complex peptide mixture of human proteins after trypsin digestion. The circled region is highly enriched for phosphopeptides.

FIGS. 7A-C show an analysis of human nuclear phosphorylation sites by LC/LC-MS/MS/MS. FIG. 7A shows Eight mg of nuclear extract from asynchronous HeLa cells were separated by SDS-PAGE. The entire gel was excised into 10 regions and proteolyzed with trypsin followed by phosphopeptide enrichment by strong cation exchange (SCX) liquid chromatography (LC). Early eluting fractions were subjected to amino acid sequence analysis by reverse-phase LC-MS/MS with data-dependent MS³ acquisition. 2,002 phosphorylation sites were identified by the Sequest algorithm, acquisition of MS³ spectra, and manual validation. FIG. 7B shows an example of a tandem mass (MS/MS) spectrum of a phosphopeptide showing a typical extensive neutral loss of phosphoric acid. FIG. 7C shows the MS/MS/MS (MS³) spectrum of the neutral loss precursor ion from panel B. Abundant fragmentation now resulted at peptide bonds permitting the unambiguous identification of this peptide from the protein, cell division cycle 2-related protein kinase 7, with a phosphorylated serine residue marked by an asterisk.

FIGS. 8A-F show classification of identified phosphorylation sites and amino acid frequencies surrounding phosphorylated serine and threonine residues. FIG. 8A shows a Venn Diagram representation of 1,833 precise sites of phosphorylation with respect to surrounding residues. Seventy seven percent of the detected phosphorylation sites could be assigned as either proline-directed or acidiphilic. FIG. 8B shows phosphorylation sites grouped by protein localization and function. The largest class of proteins detected was “unknown” (uncharacterized or hypothetical). “Other” represents known proteins not in other categories (mostly well-characterized cytosolic proteins). FIG. 8C is an intensity map showing the relative occurrence of residues flanking all phosphorylation sites. FIG. 8D is an intensity map showing the relative occurrence of residues flanking proline-directed ({pSer/pThr}—Pro ) phosphorylation sites. FIG. 8E is an intensity map showing the relative occurrence of residues flanking acidiphilic ({pSer/pThr}—Xxx—Xxx—{Asp/Glu/pSer}) sites. FIG. 8F is an intensity map showing the relative occurrence of residues flanking all other phosphorylation sites. To facilitate comparisons an intensity gradient of light to dark was used ranging from white (no occurrence) to black (high occurrence).

### DETAILED DESCRIPTION

The invention provides systems, software, methods and kits for detecting and/or quantifying phosphorylatable polypeptides and/or acetylated polypeptides in complex mixtures, such as a lysate of a cell or cellular compartment (e.g., such as an organelle). The methods can be used in high throughput assays to profile phosphoproteomes and to correlate sites and amounts of phosphorylation with particular cell states.

Unless defmed otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

“Protein”, as used herein, means any protein, including, but not limited to peptides, enzymes, glycoproteins, hormones, receptors, antigens, antibodies, growth factors, etc., without limitation. Presently preferred proteins include those comprised of at least 25 amino acid residues, more preferably at least 35 amino acid residues and still more preferably at least 50 amino acid residues.

As used herein, “a polypeptide” refers to a plurality of amino acids joined by peptide bonds. Amino acids can include D-, L-amino acids, and combinations thereof, as well as modified forms thereof. As used herein, a polypeptide is greater than about 20 amino acids. The term “polypeptide” generally is used interchangeably with the term “protein”; however, the term polypeptide also may be used to refer to a less than full-length protein (e.g., a protein fragment) which is greater than 20 amino acids.

As used herein, the term “peptide” refers to a compound of two or more subunit amino acids, and typically less than 20 amino acids. The subunits are linked by peptide bonds.

The terms “polypeptide”, and “protein” are generally used interchangeably herein to refer to a polymer of amino acid residues. As used herein a peptide is generally about 100 amino acids or less.

As used herein, a “target protein” or a “target polypeptide” is a protein or polypeptide whose presence or amount is being determined in a protein sample. The protein/polypeptide may be a known protein (i.e., previously isolated and purified) or a putative protein (i.e., predicted to exist on the basis of an open reading frame in a nucleic acid sequence).

As used herein, a “protease activity” is an activity that cleaves amide bonds in a protein or polypeptide. The activity may be implemented by an enzyme such as a protease or by a chemical agent, such as CNBr.

As used herein, “a protease cleavage site” is an amide bond which is broken by the action of a protease activity.

As used herein, the term “phosphorylation site” or “phospho site” refers to an amino acid or amino acid sequence of a natural binding.domain or a binding partner which is recognized by a kinase or phosphatase for the purpose of phosphorylation or dephosphorylation of the polypeptide or a portion thereof. A “site” additionally refers to the single amino acid which is phosphorylated or dephosphorylated. Generally, a phosphorylation site comprises as few as one but typically from about 1 to 10, about 1 to 50 amino acids, i.e., less than the total number of amino acids present in the polypeptide.

The term “agonist” as used herein, refers to a molecule that augments a particular activity, such as kinase-mediated phosphorylation or phosphatase-mediated dephosphorylation. The stimulation may be direct, or indirect, or by a competitive or non-competitive mechanism. The term “antagonist”, as used herein, refers to a molecule that decreases the amount of or duration of a particular activity, such as kinase-mediated phosphorylation or phosphatase-mediated dephosphorylation. The inhibition may be direct, or indirect, or by a competitive or non-competitive mechanism. Agonists and antagonists may include proteins, including antibodies, that compete for binding at a binding region of a member of the complex, nucleic acids including anti-sense molecules, carbohydrates, or any other molecules, including, for example, chemicals, metals, organometallic agents, etc.

The term “recombinant protein” refers to a protein which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

The term “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate.

As used herein, an “isolated organelle” or “isolated cellular compartment” refers to a membrane bound intracellular structure which is substantially removed from a cell such that a sample comprising an isolated organelle or isolated cellular compartment comprises less than 50%, less than 20%, and preferably, less than 10% cellular proteins other than those which are part of (e.g., lie within or on the membrane of the membrane bound intracellular membrane structure).

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules.

As used herein, a “labeled peptide internal standard” refers to a synthetic peptide which corresponds in sequence to the amino acid subsequence of a known protein or a putative protein predicted to exist on the basis of an open reading frame in a nucleic acid sequence and which is labeled by a mass-altering label such as a stable isotope. The boundaries of a labeled peptide internal standard are governed by protease cleavage sites in the protein (e.g., sites of protease digestion or sites of cleavage by a chemical agent such as CNBr). Protease cleavage sites may be predicted cleavage sites (determined based on the primary amino acid sequence of a protein and/or on the presence or absence of predicted protein modifications, using a software modeling program) or may be empirically determined (e.g., by digesting a protein and sequencing peptide fragments of the protein). In one aspect, a labeled peptide internal standard includes a modified amino acid residue.

“Percent identity” and “similarity” between two sequences can be determined using a mathematical algorithm (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). For example, the percent identity between two amino acid sequences can be determined using the Needleman and Wunsch algorithm (J. Mol. Biol. (48): 444453, 1970) which is part of the GAP program in the GCG software package (available at http://www.gcg.com), by the local homology algorithm of Smith & Waterman (Adv. Appl. Math. 2: 482, 1981), by the search for similarity methods of Pearson & Lipman (Proc. Natl. Acad. Sci. USA 85: 2444, 1988) and Altschul, et al. (Nucleic Acids Res. 25(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection (see, e.g., Ausubel et al., supra). Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Examplary gap weights using a Blossom 62 matrix or a PAM250 matrix, are 16, 14, 12, 10, 8, 6, or 4, while exemplary length weights are 1, 2, 3, 4, 5, or 6. The percent identity between two amino acid or nucleotide sequences also can be determined using the algorithm of E. Myers and W. Miller (CABIOS 4: 11-17, 1989) which has been incorporated into the ALIGN program (version 2.0), using a PAM120 weight residue table, a gap length penalty of 12 and a gap penalty of 4.

As used herein, “a peptide fragmentation signature” refers to the distribution of mass-to-charge ratios of fragmented peptide ions obtained from fragmenting a peptide, for example, by collision induced disassociation, ECD, LID, PSD, IRNPD, SID, and other fragmentation methods. A peptide fragmentation signature which is “diagnostic” or a “diagnostic signature” of a target protein or target polypeptide is one which is reproducibly observed when a peptide digestion product of a target protein/polypeptide identical in sequence to the peptide portion of a peptide internal standard, is fragmented and which differs only from the fragmentation pattern of the peptide internal standard by the mass of the mass-altering label. Preferably, a diagnostic signature is unique to the target protein (i.e., the specificity of the assay is at least about 95%, at least about 99%, and preferably, approaches 100%).

As used herein, the interchangeable terms “biological specimen” and “biological sample” refer to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate or extract prepared from a whole organism or a subset of its tissues, cells or component parts, or a fraction or portion thereof. The biological sample can be in any form, including a solid material such as a tissue, cells, a cell pellet, a cell extract, a biopsy, a biological fluid such as urine, blood, saliva, spinal fluid, amniotic fluid, exudate from a region of infection or inflammation, or a mouthwash containing buccal cells. In one aspect, a “biological sample” refers to a medium, such as a nutrient broth or gel in which an organism has been propagated, which contains cellular components, such as proteins or nucleic acid molecules.

As used herein, “modulation” refers to the capacity to either increase or decease a measurable functional property of biological activity or process (e.g., enzyme activity or receptor binding) by at least 10%, 15%, 20%, 25%, 50%, 100% or more; such increase or decrease may be contingent on the occurrence of a specific event, such as activation of a signal transduction pathway, and/or may be manifest only in particular cell types.

As used herein, the term “modulating the activity of a protein kinase or phosphatase” refers to enhancing or inhibiting the activity of a protein kinase or phosphatase. Such modulation may be direct (e.g. including, but not limited to, cleavage of—or competitive binding of another substance to the enzyme) or indirect (e.g. by blocking the initial production or activation of the kinase or phosphatase).

A “relational” database as used herein means a database in which different tables and categories of the database are related to one another through at least one common attribute and is used for organizing and retrieving data.

The term “external database” as used herein refers to publicly available databases that are not a relational part of the internal database, such as GenBank and Blocks.

As used herein, an “expression profile” refers to measurement of a plurality of cellular constituents that indicate aspects of the biological state of a cell. Such measurements may include, e.g., abundances or proteins or modified forms thereof.

As used herein, a “cell state profile” refers to values of measurements of levels of one or more proteins in the cell. Preferably, such values are obtained by determining the amount of peptides in a sample having the same peptide fragmentation signatures as that of peptide internal standards corresponding to the one or more proteins. A “diagnostic profile” refers to values that are diagnostic of a particular cell state, such that when substantially the same values are observed in a cell, that cell may be determined to have the cell state. For example, in one aspect, a cell state profile comprises the value of a measurement of phosphorylated p53 in a cell. A diagnostic profile would be a value that is significantly higher than the value determined for a normal cell and such a profile would be diagnostic of a tumor cell. A “test cell state profile” is a profile that is unknown or being verified.

“Diagnostic” means identifying the presence or nature of a biological state, such as a pathologic condition, e.g., cancer. Diagnostic methods differ in their sensitivity and specificity. The “sensitivity” of a diagnostic assay is the percentage of samples which test positive for the state (percent of “true positives”). Samples not detected by the assay are “false negatives.” Samples which are not from sources having the biological state and who test negative in the assay, are termed “true negatives.” The “specificity” of a diagnostic assay is 1 minus the false positive rate, where the “false positive” rate is defined as the proportion samples which are from sources which do not have the state which test positive. While a particular diagnostic method may not provide a definitive diagnosis of a biological state, it suffices if the method provides a positive indication that aids in diagnosis. The methods of the present invention preferably provide a specificity of at least 80%, more preferably at least 85%. The methods of the present invention preferably provide a sensitivity of at least 70%, more preferably at least 75%, and most preferably at least 80%.

As used herein, a processor that “receives a diagnostic profile” receives data relating to the values diagnostic of a particular cell state. For example, the processor may receive the values by accessing a database where such values are stored through a server in communication with the processor.

As used herein, “a binding partner” refers to a first molecule which can form a stable, and specific, non-covalent association with a second molecule to be bound, enabling isolation of the second molecule from a population of molecules including the second molecule. “Stable” refers to an association which is strong enough to permit complexes to form which may be isolated.

As used herein, an “antibody” refers to monoclonal or polyclonal, single chain, double chain, chimeric, humanized, or recombinant antibody, or antigen-binding portion thereof (e.g., F(ab′)2 fragments and Fab′ fragments).

As used herein, “computer readable media” or a “computer memory” refers to any media that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; digital video disc (DVDs), compact discs (CDs), hard disk drives (HDD), and magnetic tape and hybrids of these categories such as magnetic/optical storage media.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “in communication with” refers to the ability of a system or component of a system to receive input data from another system or component of a system and to provide an output response in response to the input data. “Output” may be in the form of data or may be in the form of an action taken by the system or component of the system.

As used herein, a “computer program product” refers to the expression of an organized set of instructions in the form of natural or programming language statements that is contained on a physical media of any nature (e.g., written, electronic, magnetic, optical or otherwise) and that may be used with a computer or other automated data processing system of any nature (but preferably based on digital technology). Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

Methods of Characterizing a Phosphoproteome

The invention provides methods for characterizing a phosphoproteome. The methods facilitate identification of phosphorylated proteins, identification of phosphorylation sites; quantitation of phosphorylation at one or more phosphorylation sites in a protein and determination of the biological function of phosphorylation. A phosphate group can modify serine, threonine, tyrosine, histidine, arginine, lysine, cysteine, glutamic acid and aspartic acid residues. The methods according to the invention are able to identify modifications at each of these groups and to distinguish between them.

In one aspect, the method comprises providing a sample comprising a plurality of polypeptides and separating the polypeptides according to at least one physical property. Samples that can be analyzed by method of the invention include, but are not limited to, cell homogenates; cell fractions; biological fluids, including, but not limited to urine, blood, and cerebrospinal fluid; tissue homogenates; tears; feces; saliva; lavage fluids such as lung or peritoneal ravages; and generally, any mixture of biomolecules, e.g., such as mixtures including proteins and one or more of lipids, carbohydrates, and nucleic acids such as obtained partial or complete fractionation of cell or tissue homogenates.

Sub-tissue distribution, such as in particular cells, organelles, fractions and so on also can be examined. The tissue is treated to release the individual component cell or cells; the cells are treated to release the individual component organelles and so on. Those partitioned samples then can serve as the protein source. To provide a more particularized origin of protein, specific kinds of cells can be purified from a tissue using known materials and methods. To provide proteins specific for an organelle, the organelles can be partitioned, for example, by selective digestion of unwanted organelles, density gradient centrifugation or other forms of separation, and then the organelles are treated to release the proteins therein and thereof. The cells or subcellular components are lysed as described hereinabove. Other specific techniques for isolating single cells or specific cells are known such as Emmert-Buck et al., “Laser Capture Microdissection” Science 274(5289): 998-1001 (1996).

Preferably, a proteome is analyzed. By a proteome is intended at least about 20% of total protein coming from a biological sample source, usually at least about 40%, more usually at least about 75%, and generally 90% or more, up to and including all of the protein obtainable from the source. Thus, the proteome may be present in an intact cell, a lysate, a microsomal fraction, an organelle, a partially extracted lysate, biological fluid, and the like. The proteome will be a mixture of proteins, generally having at least about 20 different proteins, usually at least about 50 different proteins and in most cases, about 100 different proteins, about 1000 different proteins, about 10,000 different proteins, about 100,000 different proteins, or more.

In one aspect, a proteome comprises substantially all of the proteins in a cell. In another preferred aspect, an organellar proteome is evaluated. For example, at least about at least about 50 different proteins and in most cases, about 100 different proteins, about 1000 different proteins, about 10,000 different proteins, about 100,000 different proteins, or more from an organelle such as a nucleus, mitochondria, chloroplast, golgi body, vacuole, or other intracellular compartment. In one preferred aspect, a complex mixture of cellular proteins is evaluated directly from a cell lysate, i.e., without any steps to separate and/or purify and/or eliminate cellular components or cellular debris. In another aspect, proteins are obtained from intracellular fractions corresponding comprising substantially purified preparations of intracellular organelles, e.g., such as cell nuclei, mitochondria, chloroplasts, golgi bodies, vacuoles, and the like.

Although the methods described herein are compatible with any biochemical, immunological or cell biological fractionation methods that reduce sample complexity and enrich for proteins of low abundance, it is a particular advantage of the method that it can be used to detect and quantitate peptides in complex mixtures of polypeptides, such as cell lysates. Unlike methods in the prior art, because the present invention detects diagnostic signatures that are highly selective for individual phosphorylatable peptides, the quantities of such peptides can be discerned even in a mixture of phosphorylated and unphosphorylated peptides of similar mass/charge ratios.

Generally, the sample will have at least about 0.01 mg of protein, at least about 0.05 mg, and usually at least about 1 mg of protein, at least about 10 mg of protein, at least about 20 mg of protein or more, typically at a concentration in the range of about 0.1-20 mg/ml. The sample may be adjusted to the appropriate buffer concentration and pH, if desired.

The physical property can include molecular weight, binding affinity for a ligand or receptor, hydrophobicity, hydrophilicity, and the like.

Preferred methods of separating polypeptides according to binding affinity include through the use of an array or substrate comprising a plurality of binding partners stably associated therewith (e.g., by attachment, deposition, etc.) for selectively binding to sample components. Suitable binding partners include, but are not limited to: cationic molecules; anionic molecules; metal chelates; antibodies; single- or double-stranded nucleic acids; proteins, peptides, amino acids; carbohydrates; lipopolysaccharides; sugar amino acid hybrids; molecules from phage display libraries; biotin; avidin; streptavidin; and combinations thereof. Generally, any molecule that has an affinity for desired sample components or which can selectively or specifically absorb a biological molecule can be used as a binding partner. Binding partners stably associated with the array may comprise a single type of molecule or functional group. In one aspect, the binding partner is a metal ion immobilized on an IMAC column.

In one preferred aspect, the plurality of polypeptides is separated at least according to molecular weight using liquid or gel-based separation on a 5-15% SDS polyacrylamide gel. For example, a cell lysate can be loaded onto a single lane gel and electrophoresed using methods known in the art to separate proteins.

In another aspect, polypeptides separated according to the at least one characteristic are divided into subsets. Inclusion in a particular subset may be based on a quality of the characteristic. For example, where the characteristic is molecular weight, polypeptides may be divided into subsets based on their molecular weights. Accordingly, polypeptides separated by gel electrophoresis may be divided into subsets by slicing the gel into fragments that are placed into separate containers (e.g., tubes) for subsequent analysis. The quality of the characteristic corresponding to each subset is recorded for later correlation with other characteristics of one or more members of the subset (e.g., such as phosphorylation state). An aliquot of a sample may be run on a parallel gel which is stained to ensure the presence/quality of proteins in the sample.

In another aspect, the subset is selected at random, merely to reduce the complexity of polypeptides within the subset in further analyses.

Polypeptides within each subset are then contact with one or more proteases to digest the polypeptides into peptides. Generally, the type of protease is not limiting. Suitable proteases include, but are not limited to one or more of: serine proteases (e.g., such as trypsin, hepsin, SCCE, TADG12, TADG14); metallo proteases (e.g., such as PUMP-1); chymotrypsin; cathepsin; pepsin; elastase; pronase; Arg-C; Asp-N; Glu-C; Lys-C; carboxypeptidases A, B, and/or C; dispase; thermolysin; cysteine proteases such as gingipains, and the like.

In one aspect of the invention, peptide fragments ending with Lys or Arg residues are produced. While trypsin is an exemplary protease, many different enzymes can be used to perform the digestion to generate peptide fragments ending with Lys or Arg residues, including but not limited to, Thrombin [EC 3.4.21.5], Plasmin [EC 3.4.21.7], Kallilkrein [EC 3.4.21.8], Acrosin [EC 3.4.21.10], and Coagulation factor Xa [EC 3.4.21.6], and the like. See, e.g., Dixon, et al., In Enzymes (3rd edition, Academic Press, New York and San Francisco, 1979).

Other enzymes known to reliably and predictably perform digestions to generate the polypeptide fragments as described in the instant invention are also within the scope of the invention. Proteases may be isolated from cells or obtained through recombinant techniques.

Chemical agents with a protease activity also can be used (e.g., such as CNBr).

Protease digestion is allowed to proceed so that peptide fragments are produced comprising N-terminal peptides, C-terminal peptides and internal peptides. The charge characteristics of the peptides will depend on the presence and nature of modifications of polypeptides from which the peptides derive.

Peptide products of this digestion are separated according to charge and enriched for phosphorylated peptides. In one aspect, peptides are also enriched for N-terminal and C-terminal peptides. N- and-C-terminal peptides can be used to generate standards for quantitating phosphorylated peptides obtained from the same protein sequence from which an N- and or C-terminal peptide derives. Alternatively or additionally, N- and C-terminal peptides can be used to validate the start and stop points of ORF's identified from genomic sequence data.

In one preferred aspect, phosphorylated peptides are enriched for by separating the plurality of peptides in a subset of polypeptides using strong cation exchange techniques.

Cation ion exchange chromatography (CEX) is a separation technique which exploits the interaction between positively charged groups on a peptide and negatively charged groups on a substrate. Because pH determines the charges on peptides, the pH of the medium in which CEX is carried out determines separation performance. CEX substrates can be grouped into 2 major types; those which maintain a negative charge on the substrate over a wide pH range (strong CEX substrates) and those which maintain a negative charge on the substrate over a narrow pH range (weak CEX). Strong cation exchange (SCX) substrates usually incorporate sulphonic acids derivatives as functional groups (e.g. Sulphonates, S-type or Sulphopropyl groups, SP-types). Suitable strong cation exchangers include, but are not limited to sulfonated cellulose, phosphorylated cellulose, sulfonated dextran, phosphorylated dextran, sulfonated polyacrylamide and phosphorylated polyacrylamide. Examples of suitable strong CEX substrates include S-Sepharose FF, SP- Sepharose FF, SP-Sepharose Big Beads (all Amersham Pharmacia Biotechnology), Fractogel EMD-SO (3)650 (M) (E.Merck, Germany), polysulfoethyl aspartamide (The Nest Group, Southborough, Mass.). In one particularly preferred aspect of the invention, the cationic substrate is poly(2-sulfoethyl aspartamide)-silica. Cation exchangers may be in a granular state, film state or liquid state, although a granular state is generally most practical, facilitating absorption and elution of peptides, while permitting reuse of the granules in a subsequent round of enrichment with a new subset of peptides. Methods of SCX are described in Peng, et al., J. Proteome Res. 2: 43-50, 2002.

Generally SCX columns comprise a methanol storage solvent for storage. The storage solvent should be flushed prior to use of the column to prevent salt precipitation. Preferably, the column is eluted with a strong buffer for at least one hour prior to its initial use. An exemplary buffer solution comprises 0.2 M monosodium phosphate and 0.3 M sodium acetate. Selectivity can be enhanced by varying the pH, ionic strength or organic solvent concentration in the mobile phase. For more strongly hydrophobic peptides, a non-ionic surfactant and/or acetonitrile comprise a suitable mobile phase modifier. Alternatively or additionally, the slope of a salt gradient used to elute peptides from the column can be modified.

At pH 3.0, amine finctional groups of peptides almost exclusively contribute to the solution charge state. The nominal charge of any peptide can be determined by adding up the number of lysine, arginine, and histidine residues, with one additional charge contributed by the N-terminus of the peptide. Tryptic peptides generally have solution charge states of 2+ because they terminate in lysine or arginine and have a free N-terninus. A solution charge state of 3+ is seen for tryptic peptides containing one histidine residue. Tryptic peptides carrying a single charge in solution at pH 3.0 are highly specialized, representing either the C-terminal peptide from a polypeptide, an N-terminal peptide that is blocked (e.g., acetylated), or a phosphorylated peptide. Peptides which elute with solution charge states of 4+ or more also represent specialized peptides, e.g., such as disulfide-linked tryptic peptides, missed cleavages, etc. SCX can be used to distinguish among these various charged states.

SCX chromatography has the advantage of removing proteases and binding peptides in the presence of accessory molecules that carry no positive charge at pH 3.0, the pH at which peptide elution typically occurs. Thus, peptide binding and elution can occur in the presence of molecules typically used in cellular extraction processes, such as SDS, detergent, urea, DTT, and the like.

In order to maximize the performance of the SCX substrate, the pH of the medium in which the separation is carried out is usually below the isoelectric point of the peptide to be bound. It is a discovery of the instant invention that at a pH of about 3, phosphorylated proteins and acetylated proteins are enriched for in initial fractions obtained from a SCX column. Accordingly, in one aspect, the method comprises selecting initial fractions enriched for modified peptides, e.g., peptides which elute preferably within the first about 100 fractions, within the first about 90 fractions, within the first about 80 fractions, within the first about 70 fractions, within the first about 60 fractions, within the first about 50 fractions, within the first about 40 fractions, about 35 fractions, within the first about 30 fractions, within the first about 25 fractions, within the first about 20 fractions, within the first about 15 fractions, within the first about 10 fractions, within the first about 5 fractions, within the first about 2 fractions, within the first about 1 fraction after contacting the column with an elution substance such as a salt solution or volatile basic.substance (e.g., , such as is ammonia, monomethylamine or dimethylamine). In one aspect, the initial fraction or a set of initial fractions (e.g., fractions 1-10, 1-1 5, 1-20, 1-25, 1-30, 1-35, 1-40, 1-45, 1-50, 1-60, 1-70, 1-80, 1-140, and any intervening increments thereof, comprise at least about 100,000 different peptides, at least about 160,000 different peptides, at least about 180,000 different peptides, at least about 190,000 different peptides, at least about 200,000 different peptides, at least about 220,000 different peptides, at least about 250, different peptides, at least about 260, 000 different peptides, at least about 280,000 different peptides, at least about 300,000 different peptides, at least about 320,000 different peptides, at least about 340,000 different peptides, at least about 360,000 different peptides, at least about 380,000 different peptides, at least about 400,000 different peptides, 420,000, at least about 440,000 different peptides, at least about 460,000 different peptides, or at least about 500,000 different peptides.

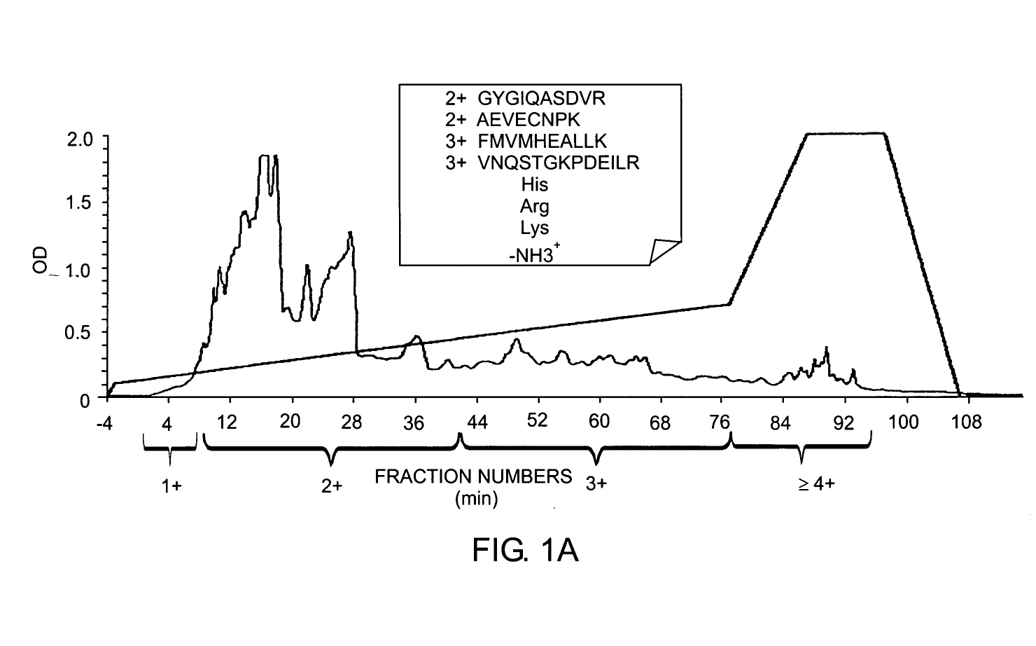

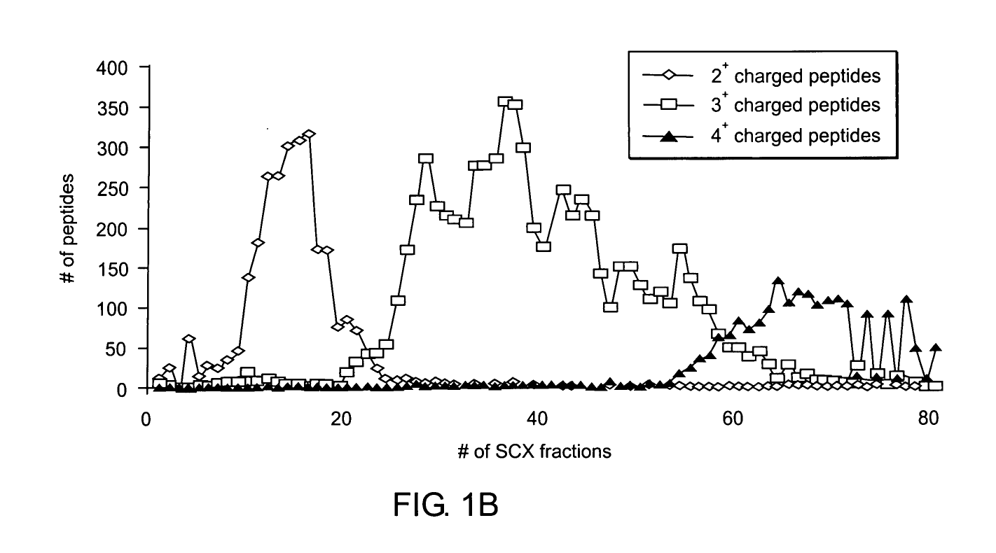

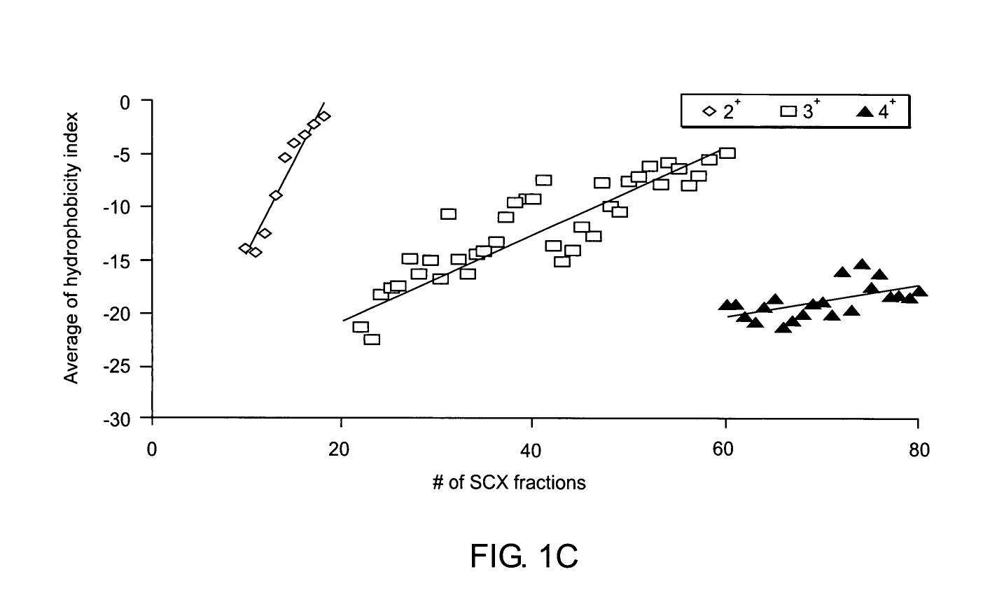

It was discovered further that, at pH 2.7, only lysines, arginines, histidines and the amino terminus of a peptide are charged. Trypsin proteolysis produces peptides with a C-terminal lysine or arginine. Thus, most tryptic peptides carry a net solution charge state of 2⁺ as shown in FIG. 1a. Because a phosphate group maintains a negative charge at acidic pH values, the net charge state of a phosphopeptide is generally only 1⁺. Interestingly, an exhaustive theoretical tryptic digest of the human protein database from NCBI produced peptides with 68% predicted to have a net charge of 2⁺ (FIG. 1b). Any of these peptides would have a net charge state of I+after a single phosphorylation event. Strong cation exchange (SCX) chromatography separates peptides based primarily on ionic charge. The SCX separation of a complex peptide mixture at pH 2.7 generated by trypsin proteolysis is shown in FIG. 1c. Phosphopeptides with a charge state of 1⁺ eluted earlier and were greatly enriched from the predominantly nonphosphorylated peptides.

The proteins eluted from the cation exchanger can be concentrated further for analysis by any suitable procedure. In one aspect, concentration is effected using reduced pressure or by heat concentration. Drying can be carried out, if necessary, after the concentration, by heat drying, spray drying or lyophilization.

Detection and Quantitation of Protein Modifications: Identifying Protein Phosphorylation Sites

In one aspect, phosphorylated peptides are evaluated to determine their identifying characteristics, e.g., such as mass, mass-to-charge (m/z) ratio, sequence, etc. Suitable peptide analyzers include, but are not limited to, a mass spectrometer, mass spectrograph, single-focusing mass spectrometer, static field mass spectrometer, dynamic field mass spectrometer, electrostatic analyzer, magnetic analyzer, quadropole analyzer, time of flight analyzer (e.g., a MALDI Quadropole time-of-flight mass spectrometer), Wien analyzer, mass resonant analyzer, double-focusing analyzer, ion cyclotron resonance analyzer, ion trap analyzer, tandem mass spectrometer, liquid secondary ionization MS, and combinations thereof in any order (e.g., as in a multi-analyzer system). Such analyzers are known in the art and are described in, for example, Mass Spectrometry for the Biological Sciences, Burlingame and Carr eds., Human Press, Totowa, N.J.).

In general, any analyzer can be used which can separate matter according to its anatomic and molecular mass. Preferably, the peptide analyzer is a tandem MS system (an MS/MS system) since the speed of an MS/MS system enables rapid analysis of low femtomole levels of peptide and can be used to maximize throughput.

In a preferred aspect, the peptide analyzer comprises an ionizing source for generating ions of a test peptide and a detector for detecting the ions generated. The peptide analyzer further comprises a data system for analyzing mass data relating to the ions and for deriving mass data relating to a phosphorylated peptide.

In one preferred aspect, peptides are analyzed by fragmenting the peptide. Fragmentation can be achieved by inducing ion/molecule collisions by a process known as collision-induced dissociation (CID) (also known as collision-activated dissociation (CAD)). Collision-induced dissociation is accomplished by selecting a peptide ion of interest with a mass analyzer and introducing that ion into a collision cell. The selected ion then collides with a collision gas (typically argon or helium) resulting in fragmentation. Generally, any method that is capable of fragmenting a peptide is encompassed within the scope of the present invention. In addition to CID, other fragmentation methods include, but are not limited to, surface induced dissociation (SID) (James and Wilkins, Anal. Chem. 62: 1295-1299, 1990; and Williams, et al., J. Amer. Soc. Mass Spectrom. 1: 413416, 1990), blackbody infrared radiative dissociation (BIRD); electron capture dissociation (ECD) (Zubarev, et al., J. Am. Chem. Soc. 120: 3265-3266, 1998); post-source decay (PSD), LID, and the like.

The fragments are then analyzed to obtain a fragment ion spectrum. One suitable way to do this is by CID in multistage mass spectrometry (MSⁿ). Traditionally used to characterize the structure of a peptide and/or to obtain sequence information, it is a discovery of the present invention, that MSⁿ provides enhanced sensitivity in methods for quantitating absolute amounts of proteins.

Preferably, peptides are analyzed by at least two stages of mass spectrometry to determine the fragmentation pattern of the peptide. More preferably, the fragmentation pattern of phosphorylated and unphosphorylated forms of the peptide is determined. Most preferably, a peptide signature is obtained in which peptide fragments corresponding to phosphorylated and unphosphorylated forms have significant differences in m/z ratios to enable peaks corresponding to each fragment to be well separated. Still more preferably, signatures are unique, i.e., diagnostic of a peptide being identified and comprising minimal overlap with fragmentation patterns of peptides with different amino acid sequences. If a suitable fragment signature is not obtained at the first stage, additional stages of mass spectrometry are performed until a unique signature is obtained.

The peptide analyzer additionally comprises a data system for recording and processing information collected by the detector. The data system can respond to instructions from processor in communication with the separation system and also can provide data to the processor. Preferably, the data system includes one or more of: a computer, an analog to digital conversion module; and control devices for data acquisition, recording, storage and manipulation. More preferably, the device further comprises a mechanism for data reduction, i.e., to transform the initial digital or analog representation of output from the analyzer into a form that is suitable for interpretation, such as a graphical display (e.g., a display of a graph, table of masses, report of abundances of ions, etc.).

The data system can perform various operations such as signal conditioning (e.g., providing instructions to the peptide analyzer to vary voltage, current, and other operating parameters of the peptide analyzer), signal processing, and the like. Data acquisition can be obtained in real time, e.g., at the same time mass data is being generated. However, data acquisition also can be performed after an experiment, e.g., when the mass spectrometer is off line.

The data system can be used to derive a spectrum graph in which relative intensity (i.e., reflecting the amount of protonation of the ion) is plotted against the mass to charge ratio (m/z ratio) of the ion or ion fragment. An average of peaks in a spectrum can be used to obtain the mass of the ion (e.g., peptide) (see, e.g., McLafferty and Turecek, 1993, Interpretation of Mass Spectra, University Science Books, Calif.).

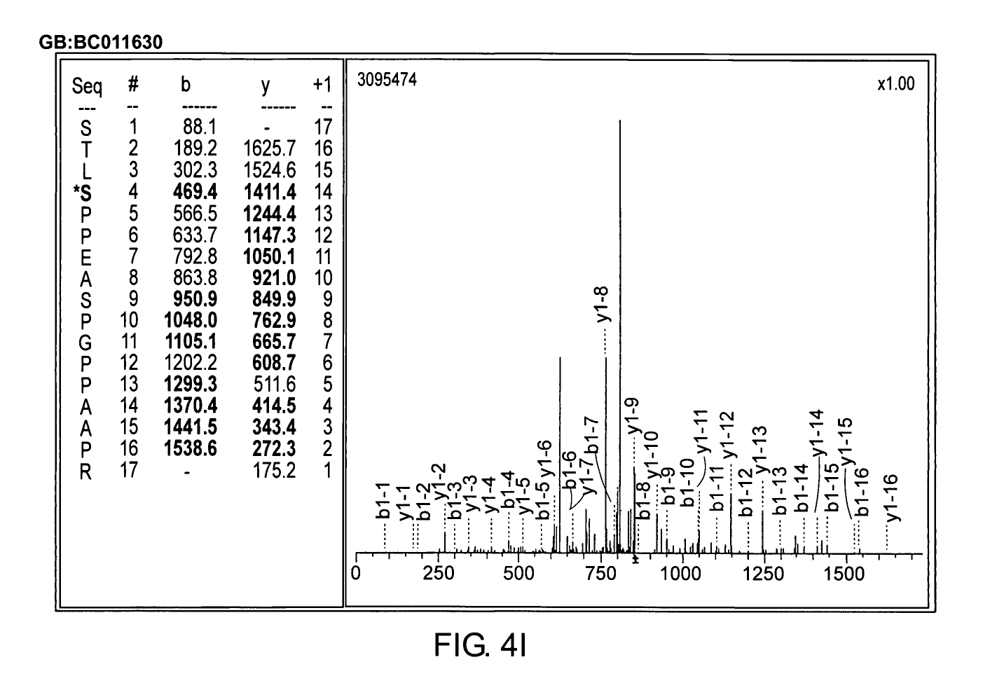

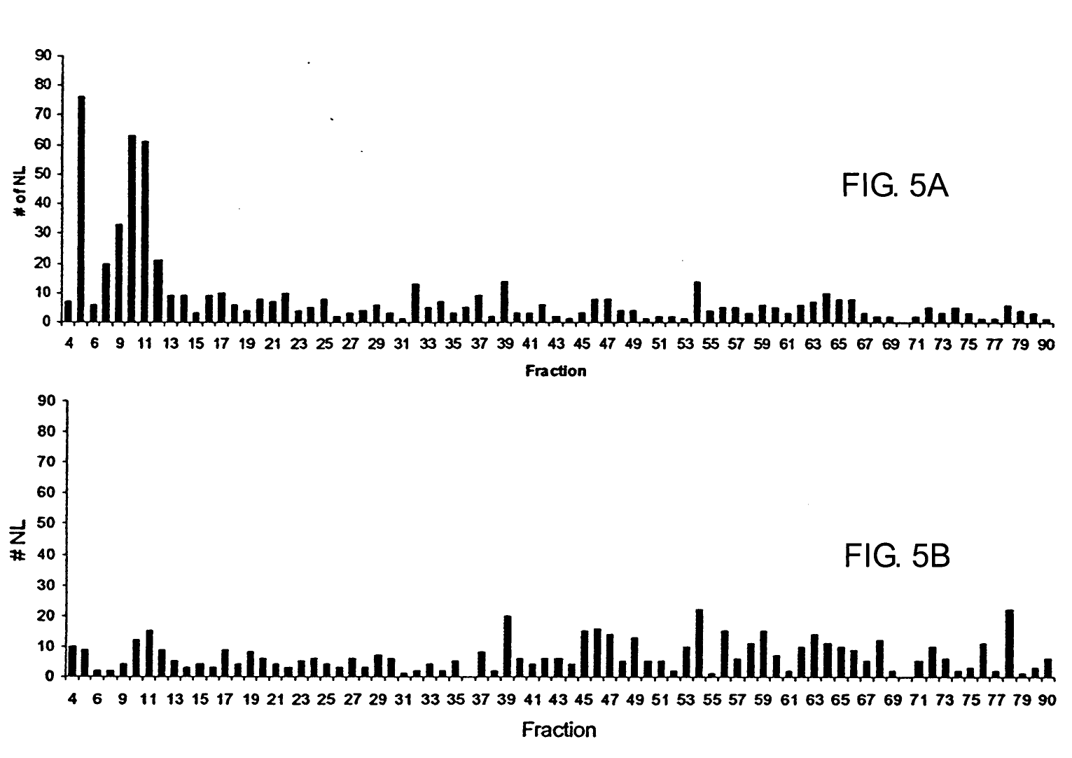

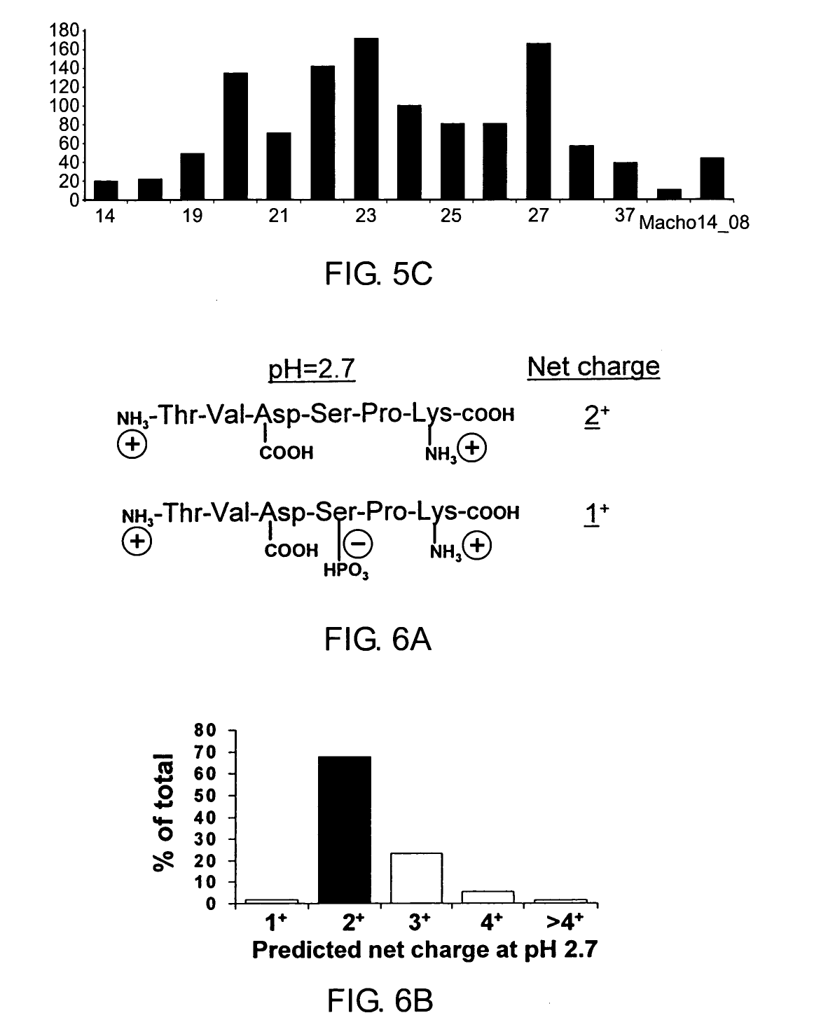

Mass spectral peaks may be used to identify protein modifications. The decomposition of a precursor ion results in a product ion and a neutral loss. Neutral Loss is the loss of a fragment that is not charged and thus not detectable by a mass spectrometer. The mass of phosphate (80) is lost as a neutral loss from a peptide. When a phosphopeptide enters a mass spectrometer, the first thing lost is the phosphate (as a neutral loss), which gives a characteristic spectrum, particularly in an ion-trap mass spectrometer. Thus neutral loss of phosphate can act as a benchmark for the presence of phosphopeptides. The control neutral loss is a random mass (in FIG. 5B, 101), and is roughly flat as expected because it represents loss arising only from noise. As can be seen in FIGS. 5A-C, neutral loss events arise more frequently in the earliest fractions collected when performing SCX according to the methods described herein.

Mass spectra can be searched against a database of reference peptides of known mass and sequence to identify a reference peptide which matches a phosphorylated peptide (e.g., comprises a mass which is smaller by the amount of mass attributable to a phosphate group). The database of reference peptides can be generated experimentally, e.g., digesting non-phosphorylated peptides and analyzing these in the peptide analyzer. The database also can be generated after a virtual digestion process, in which the predicted mass of peptides is generated using a suite of programs such as PROWL (e.g., available from ProteoMetrics, LLC, New York; N.Y.). A number of database search programs exist which can be used to correlate mass spectra of test peptides with amino acid sequences from polypeptide and nucleotide databases (i.e., predicted polypeptide sequences), including, but not limited to: the SEQUEST program (Eng, et al., J. Am. Soc. Mass Spectrom. 5: 976-89; U.S. Pat. No. 5,538,897; Yates, Jr., III, et al., 1996, J. Anal. Chem. 68(17): 534-540A), available from Finnegan Corp., San Jose, Calif.

Data obtained from fragmented peptides can be mapped to a larger peptide or polypeptide sequence by comparing overlapping fragments. Preferably, a phosphorylated peptide is mapped to the larger polypeptide from which it is derived to identify the phosphorylation site on the polypeptide. Sequence data relating to the larger polypeptide can be obtained from databases known in the art, such as the nonredundant protein database compiled at the Frederick Biomedical Supercomputing Center at Frederick, Md.

In one aspect, the amount and location of phosphorylation is compared to the presence, absence and/or quantity of other types of polypeptide modifications. For example, the presence, absence, and/or quantity of: ubiquitination, sulfation, glycosylation, and/or acetylation can be determnined using methods routine in the art (see, e.g., Rossomando, et al., 1992, Proc. Natl. Acad. Sci. USA 89: 5779-578; Knight et al., 1993, Biochemistry 32: 2031-2035; U.S. Pat. No. 6,271,037 and PCT/US03/07527). The amount and locations of one or modifications can be correlated with the amount and locations of phosphorylation sites. Preferably, such a determination is made for multiple cell states.

Data-Dependent Acquisition Of MS³ Spectra For Improved Phosphopeptide Identification

In the context of peptide mass spectrometry an MS² spectrum and MS³ spectrum represent, respectively, the measurement of fragment ions derived from a single peptide, and fragment ions derived from a single peptide fragment. Thus, if an MS² spectrum of a phosphopeptide results in a dominant phosphate-specific fragment ion, an MS³ spectrum from that dominant fragment ion can result in a more useful fragmentation pattern.

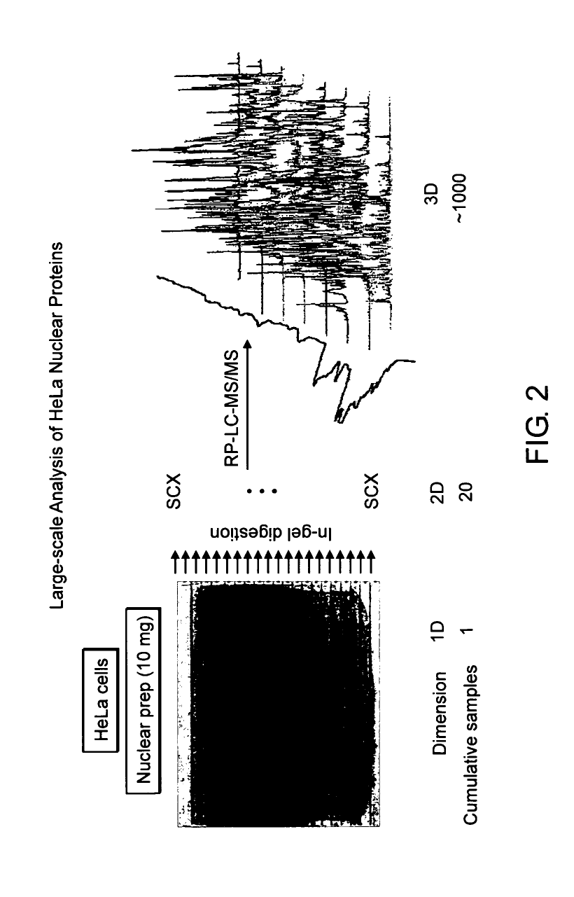

An MS³ spectrum was collected when the following conditions were met. i) The MS² spectrum revealed a significant loss of phosphoric acid (49 or 98 Da) upon fragmentation. ii) The neutral loss event was the most intense peak in the MS² spectrum. Meeting these two criteria is common for phosphopeptides but extremely unlikely for nonphosphorylated peptides. In this way, MS³ spectra were not acquired unless a phosphopeptide was suspected. An example of such a spectrum is shown in FIG. 2b. Upon fragmentation, this phosphopeptide produced mainly a single intense peak at 49 Da less than the precursor ion m/z ratio. This was recognized by software and an MS³ scan was collected by isolating and fragmenting the neutral loss fragment ion from the MS² spectrum. The result was a much richer fragmentation spectrum from which the phosphopeptide sequence could be determined including the modified residue (a serine) because the loss of phosphoric acid converted the serine residue to a dehydroalanine.

The amount of time required to collect both the MS² and MS³ spectra was less than 3 seconds.

Applications

The cell-division-cycle of the eukaryotic cell is primarily regulated by the state of phosphorylation of specific proteins, the functional state of which is determined by whether or not the protein is phosphorylated. This is determined by the relative activity of protein kinases which add phosphate and protein phosphatases which remove the phosphates from these proteins. Lack of function or improper function of either kinases or phosphatases may lead to abnormal physiological responses, such as uncontrolled cell division.

Additionally, many polypeptides such as growth factors, differentiation factors and hormones mediate their pleiotropic actions by binding to and activating cell surface receptors with an intrinsic protein tyrosine kinase activity. Changes in cell behavior induced by extracellular signaling molecules such as growth factors and cytokines require execution of a complex program of transcriptional events. To activate or repress transcription, transcription factors must be located in the nucleus, bind DNA, and interact with the basal transcription apparatus. Accordingly, extracellular signals that regulate transcription factor activity may affect one or more of these processes. Most commonly, regulation is achieved by reversible phosphorylation.

Accordingly, methods of identifying and quantifing phosphorylated proteins, polypeptides, and peptides according to the invention can be used to diagnose abnormal cellular responses including misregulated cell proliferation (e.g., cancer), to determine the activity of growth factors, differentiation factors, hormones, cytokines, transcription factors, signaling molecules and the like. Preferably, the methods are used to correlate activity with a cell state (such as a disease or a state which is responsive to an agent or condition to which a cell is exposed).

Phosphorylated proteins often comprises sequence motifs which when phosphorylated or dephosphorylated promote interaction with target proteins that modulate the activity (i.e., increase or decrease) of either the phosphorylated polypeptide or the target polypeptide. Non-limiting examples of such sequences include FLPVPEYINQSV, a sequence found in human ECF receptor, and AVGNPEYLNTVQ, a sequence found in human EGF receptor, both of which are autophosphorylated growth factor receptors which stimulate the biochemical signaling pathways that control gene expression, cytoskeletal architecture and cell metabolism, and which interact with the Sen-5 adaptor protein; the p53 sequence EPPLSQEAFADLWKK that when phosphorylated prevents the interaction, and subsequent inactivation of p53 by MDM2. In one aspect, the methods of the invention are used to characterize the frequency of such sequence motifs in a phosphoproteome correlating with a particular cell state. In another aspect, the methods of the invention are used to identify and characterize novel sequence motifs and to further correlate the phosphorylation of such motifs with the activity of a known or novel kinase.

Knowledge of phosphorylation sites can be used to identify compounds that modulate particular phosphorylated polypeptides (either preventing or enhancing phosphorylation, as appropriate, to normalize the phosphorylation state of the polypeptide). Thus, in one aspect, the method described above may further comprise contacting a first cell with a compound and comparing phosphorylation sites/amounts identified in the first cell with phosphorylation sites/amounts in a second cell not contacted with the compound. Suitable cells that may be tested include, but are not limited to: neurons, cancer cells, immune cells (e.g., T cells), stem cells (embryonic and adult), undifferentiated cells, pluripotent cells, and the like. In one preferred aspect, patterns of phosphorylation are observed in cultured cells, capable of transformation to an oncogenic state.

The invention additionally provides a method of screening for a candidate modulator of enzymatic activity of a kinase or a phosphatase, the method comprising contacting a test sample comprising a kinase or phosphatase and a plurality of proteins including a protein comprising a peptide sequence identified as described above, contacting the plurality of proteins with an agent comprising a protease activity, thereby generating a plurality of peptide digestion products, and quantitating the amount of phosphorylated peptide in the sample. The level of phosphorylated peptide in the test sample is compared to levels in a control sample comprising known activities of the kinase/phosphatase to identify candidate modulators which either decrease or increase the activities relative to the baseline established by the control sample and/or which alters the site of phosphorylation in a polypeptide. In one aspect, the method is used to identify an agonist of a kinase or phosphatase. In another aspect, the method is used to identify an antagonist of a phosphatase or kinase.

Compounds which can be evaluated include, but are not limited to: drugs; toxins; proteins; polypeptides; peptides; amino acids; antigens; cells, cell nuclei, organelles, portions of cell membranes; viruses; receptors; modulators of receptors (e.g., agonists, antagonists, and the like); enzymes; enzyme modulators (e.g., such as inhibitors, cofactors, and the like); enzyme substrates; hormones; nucleic acids (e.g., such as oligonucleotides; polynucleotides; genes, cDNAs; RNA; antisense molecules, ribozymes, aptamers), and combinations thereof. Compounds also can be obtained from synthetic libraries from drug companies and other commercially available sources known in the art (e.g., including, but not limited, to the LeadQuest® library) or can be generated through combinatorial synthesis using methods well known in the art.

Compounds identified as modulating agents are used in methods of treatment of pathologies associated with abnormal sites/levels of phosphorylation. For administration to a patient, one or more such compounds are generally formulated as a pharmaceutical composition. Preferably, a pharmaceutical composition is a sterile aqueous or non-aqueous solution, suspension or emulsion, which additionally comprises a physiologically acceptable carrier (i.e., a non-toxic material that does not interfere with the activity of the active ingredient). More preferably, the composition also is non-pyrogenic and free of viruses or other microorganisms. Any suitable carrier known to those of ordinary skill in the art may be used. Representative carriers include, but are not limited to: physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

Routes and frequency of administration, as well doses, will vary from patient to patient. In general, the pharmaceutical compositions is administered intravenously, intraperitoneally, intramuscularly, subcutaneously, intracavity or transdermally. Between I and 6 doses is administered daily. A suitable dose is an amount that is sufficient to show improvement in the symptoms of a patient afflicted with a disease associated an aberrant phosphorylation state. Such improvement may be detected by monitoring appropriate clinical or biochemical endpoints as is known in the art. In general, the amount of modulating agent present in a dose, or produced in situ by DNA present in a dose (e.g., where the modulating agent is a polypeptide or peptide encoded by the DNA), ranges from about 1 μg to about 100 mg per kg of host. Suitable dose sizes will vary with the size of the patient, but will typically range from about 10 mL to about 500 mL for 10-60 kg animal. A patient can be a mammal, such as a human, or a domestic animal.

In another aspect, the phosphorylation states (e.g., sites and amount of phosphorylation) of first and second cells are evaluated. In one aspect, the second cell differs from the first cell in expressing one or more recombinant DNA molecules, but is otherwise genetically identical to the first cell. Alternatively, or additionally, the second cell can comprise mutations or variant allelic forms of one or more genes. In one aspect, DNA molecules encoding regulators of a phosphorylatable protein can be introduced into the second cell (e.g., such as a kinase or a phosphatase) and alterations in the phosphorylation state in the second cell can be determined. DNA molecules can be introduced into the cell using methods routine in the art, including, but not limited to: transfection, transformation, electroporation, electrofusion, microinjection, and germline transfer.

Stable isotope labeling with amino acids in cell culture, or SILAC, also is a valuable proteomic technique. Ong, S.E., et al. (2002), Methods 29, 124-130;. Ong, et al. (2003). J. Proteome Res. 2, 173-181. Using SILAC in combination with the methods of the present invention can provide a powerful identification tool. Cells representing two biological conditions can be cultured in amino acid-deficient growth media supplemented with ¹²C- or ¹³C-labeled amino acids. The proteins in these two cell populations effectively become isotopically labeled as “light” or “heavy.” Upon isolation of proteins from these cells, samples can then be mixed in equal ratios and processed using conventional techniques for tandem mass spectrometry. Because corresponding light and heavy peptides from the same protein will coelute during chromatographic separation into the mass spectrometer, relative quantitative information can be gathered for each protein by calculating the ratio of intensities of the two peaks produced in the peptide mass spectrum (MS scan). Furthermore, sequence data can be acquired for these peptides by fragment analysis in the product ion mass spectrum (MS/MS scan) and used for accurate protein identification. Finally, when more than one peptide is identified from the same protein, the quantification is redundant, providing increased confidence in both the identification and quantification of the protein.

System for Analysis of Phosphoproteomes

The present invention also provides a system and software for facilitating the analysis of phosphoproteomes. The invention provides a system that comprises a relational database which stores mass spectral data relating to phoshorylation states for a plurality of proteins in a proteome. The system further comprises a data management program for correlating phosphorylation states to the source of the proteome, e.g., a cell or tissue extract, a patient group, etc.

In one aspect, the data management program comprises a data analysis program for identifying similarities of features of mass spectral signatures for one or more peptides in a plurality of peptides with mass spectral signatures for known peptides. In another aspect, the data analysis program identifies the peptide sequences for one or more peptides in the plurality of peptides. In still another aspect, the plurality of peptides is a mixture of labeled peptides, a first set of peptides labeled with a first label and a second set of peptides labeled with a second label. In a further aspect, the first label has a first mass and the second label has a second, different mass. Preferably, the data analysis system comprises a component for determining the relative abundance of a first labeled peptide with a second labeled peptide. The system is connectable to one or more external databases through a network server.

The invention also provides a method for storing peptide data to a database. The method comprises acquiring mass spectral signatures for one or more peptides in a plurality of peptides. The one or more peptides exist in a phosphorylated form in one or more cells having a cell state (e.g., a differentiation state, an association with a disease or response to an abnormal physiological condition, response to an agent, and the like). The signatures are stored in a database and correlated with the presence or absence of cell state. Preferably, pairs of signatures associated with both the phosphorylated and unphosphorylated states of the peptides are stored in the database. In one aspect, the mass spectrum signatures are obtained from mass analytical techniques, as described above.

The relational database may comprise a plurality of table or fields that may be interrelated via associations to facilitate searching the database. The database may comprise an object-oriented database, flat file database, data structures comprising linked lists, binary trees and the like. In one aspect, the database comprises a reference collection of mass spectral signatures corresponding to pairs of phosphorylated and unphosphorylated peptides comprising otherwise identical amino acid residues.

Preferably, the system further comprises a data management system. The data management system comprises a data analysis module which preferably interacts with instrumentation (e.g., such as a mass spectrometer) used to determine data features of the phosphorylated peptides obtained from strong cation exchange as described above. The data analysis system identifies peptide constituents from fractions obtained from SCX enriched for phosphorylated peptides and processes the data to obtain sequence information. Functions of the data analysis system include organizing data output, transforming or changing the format of data output, and performing statistical treatment of data. Preferably, the data analysis system interacts with the system database to organize, categorize and store data output comprising peptide signatures of phosphorylatable peptides.

In one aspect, the data analysis system preferably executes computer program code to identify peptides by comparison of mass spectral data with the database of mass spectral signatures. One such program for determining the identity of a peptide by matching tandem mass spectrum data with stored peptide spectra is the SEQUEST peptide identification program developed at the University of Washington (http://www.washington.edu). Information on the SEQUEST program and system can be found on the Internet at http://thompson.mbt.washington.edu-.

Peptide-correlated output files containing the putative identities of the peptides determined from the spectral data analysis are then returned to the data analysis system for further processing such as correlation with a biological state relating to the proteome from which the peptides were derived (e.g., such as a disease state).

In one aspect, the data analysis system communicates with the system database by way of a communication medium, such as a network server. For example, the system comprises functionality for sending and receiving data through a suitable means, such as a TCP/IP based protocol. The communication medium may additionally provide accessibility to other external databases, e.g., such as genomic databases, pharmacological databases, patient databases, proteomic databases, and the like, such as GenBank, SwissProt, Entrez, PubMed, and the like, to acquire other information which may be associated with the peptides which may be added to the system database.

In another aspect, the data analysis system base identifies peaks or intensity curves corresponding to resolved peptides in a mass spectrum obtained from proteome analysis. The data analysis system further quantitates the amount of a phosphorylatable peptide associated with a particular mass spectral peak. Preferably, the system compares peak data corresponding to the same peptide in a plurality of different proteomes associated with different cell states. The results of such calculations are stored in the system database.

Data obtained from such analyses can be stored in fields of tables comprising the relational database and used to identify differences in the phosphoproteomes of two or more biological samples. In one aspect, for a cell state determined by the differential expression of at least one phosphorylatable protein, a data file corresponding to the cell state will minimally comprise data relating to the mass spectra observed after peptide fragmentation of a peptide internal standard diagnostic of the protein. Preferably, the data file will include a data field for a value corresponding to the level of protein in a cell having the cell state.

For example, a tumor cell state is associated with the overexpression of p53 (see, e.g., Kern, et al., 2001, Int. J. Oncol. 21(2): 243-9). The data file will comprise mass spectral data observed after fragmentation of a labeled peptide internal standard corresponding to a subsequence of p53. Preferably, the data file also comprises a value relating to the level of p53 in a tumor cell. The value may be expressed as a relative value (e.g., a ratio of the level of p53 in the tumor cell to the level of p53 in a normal cell) or as an absolute value (e.g., expressed in nM or as a % of total cellular proteins). Most preferably, the data file comprises data relating to the phosphorylation state of the peptide (e.g., presence and amount of phosphorylation). Accordingly, in another aspect, one or more data fields may exist defining one or more phosphorylation sites for a protein, as well as data fields for defining an amount of protein in the sample phosphorylated at a given site.

These tables can be generated using database programming language known in the art, including, but not limited to, SQL or MySQL, in order to permit the fields and information stored in these Tables to be flexibly associated. Preferably, organization of data in the database permits search, query, and processing routines implemented by the data analysis system to associate mass spectrum peaks with one or more attributes of a protein such as amino acid sequence, phosphorylation state, mass, mass-to-charge ratio, amount of protein in a sample, and also preferably with one or more characteristics of a sample from which the mass spectrum peaks derive.

Such characteristics include characteristics relating to the sample source, including, but not limited to: presence of a disease; absence of a disease; progression of a disease; risk for a disease; stage of disease; likelihood of recurrence of disease; a genotype; a phenotype; exposure to an agent or condition; a demographic characteristic; resistance to agent, and sensitivity to an agent (e.g., responsiveness to a drug). In one aspect, the agent is selected from the group consisting of a toxic substance, a potentially toxic substance, an environmental pollutant, a candidate drug, and a known drug. The demographic characteristic may be one or more of age, gender, weight; family history; and history of preexisting conditions.

The use of the relational database provides a means of interrelating data obtained from a plurality of different proteome evaluations. Preferably, database records are configured for automated searching and extraction of data in response to queries for proteins having similar data fields. In one aspect, data analysis includes determining a correlation coefficient or confidence score which is used to order the results based on the degree of confidence with which the peptide identification and/or comparison is made. Correlation coefficients may then be stored in the database. While correlation coefficients are usually scalar numbers between 0.0 and 1.0, correlation data may alternatively comprise correlation matrices, p-values, or other similarity metrics

Object-oriented databases, which are also within the scope of the invention. Such databases include the capabilities of relational databases but are capable of storing many different data types including images of mass spectral peaks. See, e.g., Cassidy, High Performance Oracle8 SQL Programming and Tuning, Coriolis Group (March 1998), and Loney and Koch, Oracle 8: The Complete Reference (Oracle Series), Oracle Press (September 1997), the contents of which are hereby incorporated by reference into the present disclosure.

Neural network analysis of a spectrum can be performed to aid in the identification of proteomic differences and to determine correlations between these differences and one or more sample characteristic. In a neural network processing program, information is analyzed by methods such as pattern recognition or data classification. The neural network is an adaptive system that “learns” or creates associations based on previously encountered data input. Preferably rules and output of neural network analysis are also stored within the database, permitting the database to grow dynamically as more and more phosphoproteomes are evaluated.

Classification models and other pattern recognition methods can be used to identify phosphorylatable proteins that are diagnostic of at least one characteristic of a sample source. Classification models can be trained using the output from analysis of multiple samples to classify phosphorylated proteins into classes in which different phosphorylated proteins are weighted according to their ability to be diagnostic of a characteristic of a sample from which the proteins derive (e.g., such as the presence of a disease in a sample source). Classification methods may be either supervised or unsupervised. Supervised and unsupervised classification processes are known in the art and reviewed in Jain, IEEE Transactions on Pattern Analysis and Machine Intelligence 22 (1): 4-37, 2000, for example. Data mining systems utilizing such classification methods are known in the art.

Computer program code for data analysis may be written in programming language known in the art. Preferred languages include C/C++, and JAVA®. In one aspect, methods of this invention are programmed in software packages which allow symbolic entry of equations, high-level specification of processing, and statistical evaluations.

In one aspect, the system comprises an operating system in communication with each of the computer memory comprising the database and the computer memory comprising the data analysis system (the two may be the same or different). The operating system may be any system known in the art such as UNIX or WINDOWS. Preferably, the system further includes any hardware and software necessary for generating a graphical user interface on at least one user device connectable to the network using a communications protocol, such as a TCIP/IP protocol. In one aspect, the at least one user device is a wireless device.

The user device does not need to have computing power comparable to that of the database server and/or the data analysis server (the two may be the same or different servers); however, preferably, the user device is capable of displaying multiple graphical windows to a user.

The invention also provides a method for correlating a cell state associated with the expression profile of a phosphorylatable protein with the expression of a test protein using system as described above. The expression profile of the phosphorylatable protein comprises information relating to at least the phosphorylation state of at least one phosphorylation site of the phosphorylatable protein in a sample. The profile further may comprise information relating to one or more of: levels of the phosphorylatable protein and information relating to a modification of at least one other modifiable site (e.g., such as information relating to phosphorylation at a second phosphorylation site). The method is implemented by a system processor in communication with a database and data analysis system as described above. Preferably, the system processor is further in communication with a graphical user interface allowing a user to selectively view information relating to a diagnostic fragmentation signature and to obtain information about a cell state. The interface may comprise links allowing a user to access different portions of the database by selecting the links (e.g. by moving a cursor to the link and clicking a mouse or by using a keystroke on a keypad). The interface may additionally display fields for entering information relating to a sample being evaluated.

Reagents and Kits

The invention additionally provides kits for rapid and quantitative analysis of phosphoproteins in a sample. In one aspect, a kit comprises pairs of peptides identical except for the presence of phosphorylation at one or more amino acid residues of the peptides. Preferably, one or both members of the pair comprises a label. In one aspect, the label comprises a stable isotope. Suitable isotopes include, but are not limited to, ²H, ¹³C, ¹⁵N, ¹⁷O, ¹⁸O, or ³⁴S. In another aspect, pairs of peptide internal standards are provided, comprising identical peptide portions but distinguishable labels, e.g., peptides may be labeled at multiple sites to provide different heavy forms of the peptide. Pairs of peptide internal standards corresponding to phosphorylated and unphosphorylated peptides also can be provided.

In one aspect, a kit comprises peptide internal standards comprising different peptide subsequences from a single protein. In another aspect, the kit comprises peptide internal standards corresponding to sets of related proteins, e.g., such as proteins involved in a molecular pathway (a signal transduction pathway, a cell cycle, etc), or which are diagnostic of particular disease states, developmental stages, tissue types, genotypes, etc. Peptide internal standards corresponding to a set may be provided in separate containers or as a mixture or “cocktail” of peptide internal standards.

In one aspect, a plurality of peptide internal standards representing a MAPK signal transduction pathway is provided. Preferably, the kit comprises at least two, at least about 5, at least about 10 or more, of peptide internal standards corresponding to any of MAPK, GRB2, mSOS, ras, raf, MEK, p85, KHS1, GCK1, HPK1, MEKK 1-5, ELK1, c-JUN, ATF-2, 3APK, MLK1-4, PAK, MKK, p38, a SAPK subunit, hsp27, and one or more inflammatory cytokines.

In another aspect, a set of peptide internal standards is provided which comprises at least about two, at least about 5 or more, of peptide internal standards which correspond to proteins selected from the group including, but not limited to, PLC isoenzymes, phosphatidylinositol 3-kinase (PI-3 kinase), an actin-binding protein, a phospholipase D isoform, (PLD), and receptor and nonreceptor PTKs.

In another aspect, a set of peptide internal standards is provided which comprises at least about 2, at least about 5, or more, of peptide internal standards which correspond to proteins involved in a JAK signaling pathway, e.g., such as one or more of JAK 1-3, a STAT protein, IL-2, TYK2, CD4, IL-4, CD45, a type I interferon (IFN) receptor complex protein, an IFN subunit, and the like.

In a further aspect, a set of peptide internal standards is provided which comprises at least about 2, at least about 5, or more of peptide internal standards which correspond to cytokines. Preferably, such a set comprises standards selected from the group including, but not limited to, pro-and anti-inflammatory cytokines (which may each comprise their own set or which may be provided as a mixed set of peptide internal standards).

In still another aspect, a set of peptide internal standards is provided which comprises a peptide diagnostic of a cellular differentiation antigen or CD. Such kits are useful for tissue typing.

Peptide internal standards may include peptides corresponding to one or more of the peptides listed in the tables herein.

In one aspect, the peptide internal standard comprises a label associated with a phosphorylated amino acid. In another aspect, a pair of reagents is provided, a peptide internal standard corresponding to a modified peptide and a peptide internal standard corresponding to a peptide, identical in sequence but not modified.

In another aspect, one or more control peptide internal standards are provided. For example, a positive control may be a peptide internal standard corresponding to a constitutively expressed protein, while a negative peptide internal standard may be provided corresponding to a protein known not to be expressed in a particular cell or species being evaluated. For example, in a kit comprising peptide internal standards for evaluating a cell state in a human being, a plant peptide internal standard may be provided.

In still another aspect, a kit comprises a labeled peptide internal standard as described above and software for analyzing mass spectra (e.g., such as SEQUEST).

Preferably, the kit also comprises a means for providing access to a computer memory comprising data files storing information relating to the diagnostic fragmentation signatures of one or more peptide internal standards. Access may be in the form of a computer readable program product comprising the memory, or in the form of a URL and/or password for accessing an internet site for connecting a user to such a memory. In another aspect, the kit comprises diagnostic fragmentation signatures (e.g., such as mass spectral data) in electronic or written form, and/or comprises data, in electronic or written form, relating to amounts of target proteins characteristic of one or more different cell states and corresponding to peptides which produce the fragmentation signatures.

The kit may further comprise expression analysis software on computer readable medium, which is capable of being encoded in a memory of a computer having a processor and capable of causing the processor to perform a method comprising: determining a test cell state profile from peptide fragmentation patterns in a test sample comprising a cell with an unknown cell state or a cell state being verified; receiving a diagnostic profile characteristic of a known cell state; and comparing the test cell state profile with the diagnostic profile.

In one aspect, the test cell state profile comprises values of levels of phosphorylated peptides in a test sample that correspond to one or more peptide internal standards provided in the kit. The diagnostic profile comprises measured levels of the one or more peptides in a sample having the known cell state (e.g., a cell state corresponding to a normal physiological response or to an abnormal physiological response, such as a disease).

Preferably, the software enables a processor to receive a plurality of diagnostic profiles and to select a diagnostic profile that most closely resembles or “matches” the profile obtained for the test cell state profile by matching values of levels of proteins determined in the test sample to values in a diagnostic profile, to identify substantially all of a diagnostic profile which matches the test cell state profile.

In another aspect, the kit comprises one or more antibodies which specifically react with one or more peptides listed in the tables herein. In one aspect, a kit is provided which comprises an antibody which recognizes the phosphorylated form of a peptide listed in Table I but which does not recognize the unphosphorylated form. Preferably, the antibody does not universally recognize phosphorylated proteins, i.e., the antibody also specifically recognizes the amino acid sequence of the peptide rather than recognizing all peptides comprising phosphotyrosine. In one aspect, pairs of antibodies are provided - an antibody which recognizes the phosphorylated form of a peptide and not the unphosphorylated form and an antibody which recognizes the unphosphorylated form. In another aspect, the invention provides an array of antibodies specific for different phosphorylation states of a plurality of proteins in a phosphoproteome. The array can be used to monitor kinase activity and/or phosphatase activity in a phosphoproteome and as a means of evaluating the activity of one or more proteins in a cellular pathway such as a signal transduction pathway. The presence of phosphorylated proteins and level of reactivity of the antibodies can be used to monitor the site specificity and amount of phosphorylation in a sample.

Panels of antibodies can be used simultaneously to perform the analysis (e.g., by using antibodies comprising distinguishable labels). Panels of antibodies also can be used in parallel or in sequential assays. Therefore, in one preferred aspect, a kit according to the invention comprises a panel of antibodies comprising antibodies specific for phosphorylated peptidestpolypeptides phosphorylated at one or more sites.

The presence, absence, level, and/or site-specificity of other types of modifications, such as ubiquitination, also can be determined along with the presence, absence, level and/or site specificity of phosphorylation.

### EXAMPLES

The invention will now be further illustrated with reference to the following example. It will be appreciated that what follows is by way of example only and that modifications to detail may be made while still falling within the scope of the invention.

### Example 1

Tandem mass spectrometry (MS/MS) provides the means to determine the amino acid sequence identity of peptides directly from complex mixtures (Peng and Gygi, J. Mass Spectrometry 36: 1083-1091, 2001). In addition, the precise sites of modifications (e.g., acetylation, phosphorylation, etc.) to amino acid residues within the peptide sequence can be determined.

Organelle-specific proteomics provides the ability to i) more comprehensively determine the components by enriching for proteins of lower abundance, ii) study mature (fuinctional) protein, and iii) evaluate proteomics within the boundaries of cellular compartmentalization. In the present example, the isolation, separation, and large-scale amino acid sequence analysis of the HeLa cell nucleus is described. Nuclear proteins were separated by preparative SDS-PAGE. Twenty gel slices were proteolyzed with trypsin and separated by off-line strong cation exchange (SCX) chromatography and fraction collection. Each fraction was subsequently analyzed via an automated vented column approach (Licklider, et al., Anal. Chem. 74: 3076-3083, 2001) by nano-scale microcapillary LC-MS/MS in a 2-hour gradient. The analysis of slices 9 and 14 is discussed further below.

### SDS-PAGE Separation Of Nuclear Protein.

HeLa cells were harvested and nuclear protein obtained as described (McCraken, et. al., Genes and Dev. 11: 3306-3318, 1997). Ten mg of nuclear protein was separated on a 10% polyacrylamide preparative gel with a 4 cm stack. The gel was then lightly stained with Coomassie and cut into 20 slices for in-gel digestion with trypsin as described. Following digestion, complex peptide extracts were dried in a speed-vac and stored at −80° C.

### SCX Chromatography With Fraction Collection

For the SCX chromatography (Alpert and Andrews, J. Chromatogr. 443: 85-96, 1988), a commercially packed 2.1 mm×150 mm polysulfoethyl aspartamide column (PolyLC, Columbia, Md.) was used with an in-line guard column of the same material. Buffer A was 5 mM KH₂PO₄/25% acetonitrile (ACN), pH 2.7; Buffer B was the same as A with 350 mM KCl added. Following setup of the HPLC with the correct buffers and column, the flow rate was set to 200 μl/min, and a blank gradient was acquired followed by an analysis of standard peptides. A shallow gradient in the area from 5% to 35% buffer B was implemented. The acidified peptide sample was loaded onto the column and 200 μl fractions were collected every minute. Eighty fractions were collected from the SCX analysis of both Slice 9 and 14. Following this stage of analysis, fractions were reduced in volume to -50-100 μl by centrifugal evaporation in order to remove most of the acetonitrile permitting peptides to adsorb to the RP column.

### RP Chromatography Of SCX Chromatography Fractions And Identification Of Protein

All fractions from slice 9 and 14 were analyzed in a completely automated fashion using a-vented column approach (Licklider, et al., 2001, supra). Sample was loaded via an Endurance autosampler (Michrom BioResources, Inc) onto a 75 micron i.d. V-column. A gradient was developed by a Surveyor HPLC (ThermoFinnigan) with on-line elution into an ion trap mass spectrometer (LCQ-DECA, ThermoFinnigan) as described (Peng and Gygi, 2001, supra). Approximately 4000 MS/MS spectra were collected from each 2 hr analysis. All tandem mass spectra were searched against the human database (ftp://ftp.ncbi.nih.gov/blast/db/FASTA/) with the Sequest algorithm (Eng, et al., J. Am. Soc. Mass Spectrometry 5: 976-989, 1994).

Peptides were searched with no enzyme specificity and oxidized methionines and modified cysteines were considered. Peptide matches were filtered according to the following criteria: a returned peptide must be 1) fully tryptic, 2) have an Xcorr of 2.0, 1.8, and 3.0 or greater for singly, doubly, and triply charged peptides respectively, and 3) have a delta-correlation of 0.08 or greater. Next, peptides meeting this criteria were examined for redundancy within the database using a new algorithm named Dredge. Dredge makes a second pass through the database in an attempt to untangle the relationship between peptide sequence and protein identity. In addition, Dredge calculates the minimum (and maximum) number of proteins from which the peptide set identified could have originated. The minimum number of proteins is the value reported here. Non-unique peptides (peptides belonging to one or more proteins) were assigned to the protein with the largest number of peptides. Finally, proteins identified by only a single peptide were manually verified (Peng, et al., 2003, A proteomics approach to understanding protein ubiquitination. Nat. Biotech. In press.; Peng, et al., J. Proteome Res. 2: 43-50, 2002).

Massive separation of nuclear proteins was obtained. More than 2000 proteins were identified from the analysis of two gel regions. Additionally, modified peptides (i.e., phosphorylated and acetylated proteins) were also found in abundance. The analysis of the remaining regions should provide nearly universal coverage of nuclear proteins.

TABLE 1
Summary Of The Analysis Of Slice 9 And Slice 14
From The SDS-PAGE Gel.

| | | | |
|---|---|---|---|
| # fractions | 60 | 80 | 140 |
| # MS/MS | 189,000.0 | 266,000 | 455,000 |
| # Total peptides | 10256 | 49591 | 59857 |
| # Unique proteins | 939 | 1963 | 2902 |
| Average MW | 97.3 | 49.7 | N/A |

### Example 2

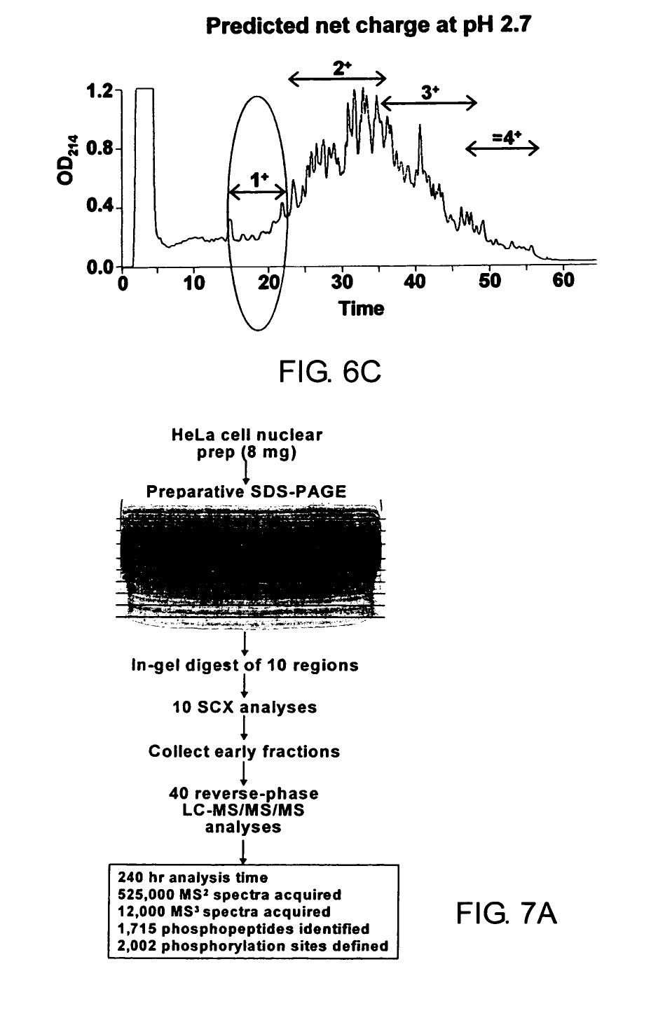

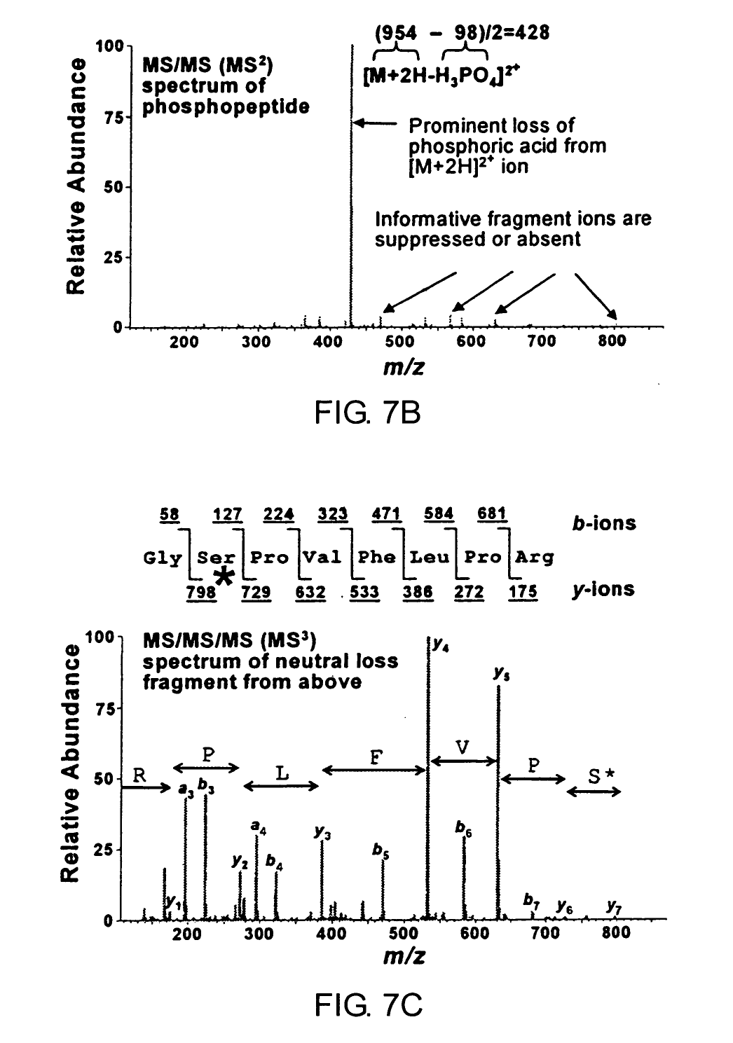

In this experiment, the characterization of phosphoproteins from asynchronous HeLa cells was performed. Because of the complexity of the sample, the proteins present in a nuclear fraction were examined and a preparative SDS-PAGE separation was applied to allow milligram quantities of starting protein (FIG. 6A). The entire gel was excised into 10 regions and proteolyzed with trypsin followed by phosphopeptide enrichment by SCX chromatography. Early-eluting fractions were subjected to further analysis by reverse-phase liquid chromatography with on-line sequence analysis by tandem mass spectrometry (LC-MS/MS).

More than 12,000 MS³ spectra were also acquired during the course of the experiment and used to help compliment database searches and manual interpretation of phosphorylation sites.

In total, 2,002 different phosphorylation sites were identified by the Sequest algorithm and each site was manually confirmed using in-house software by three different people. Matches were only deemed correct when they met exacting criteria such as the presence of intense proline-directed fragment ions, possession of the correct net solution charge state and good agreement in molecular weight of the parent protein and the region excised from the gel. The entire list of 2,002 sites is provided in Table 4.

### Methods

### HeLa Cell Nuclear Preparation, Preparative SDS-PAGE Separation and In-Gel Proteolysis

HeLa cell nuclear preparation was as described. Dignam, J. D., et al., Nucleic Acids Res 11, 1475-89 (1983). Protein (8 mg) was separated by a preparative SDS-PAGE gradient (5-15%) gel. The gel was stopped when the buffer front reached 4 cm and stained with coomassie. The entire gel was then cut into ten regions, diced into small pieces (˜1 mm³), and placed in 15 ml falcon tubes. In-gel digestion with trypsin proceeded as described but with larger volumes. Shevchenko, A., et al., Analytical Chemistry 68, 850-8 (1996). Extracts were completely dried in a speed vac and stored at −20° C.

### Strong Cation Exchange (SCX) Chromatography

Extracted peptides were redissolved in 500 μl SCX Solvent A immediately prior to analysis. Tryptic peptides were separated at pH 2.7 by SCX chromatography using a 3.0 mm×20 cm column (Poly-LC) containing 5 μm polysulfoethyl aspartamide beads with a 200 Å pore size as described. Peng, J., et al., J Proteome Res 2, 43-50 (2003). This column provided the best retention of singly-charged phosphopeptides. Fractions were collected every minute during a 60 minute gradient. Four fractions spanning the early-eluting peptides were desalted offline and completely dried. Rappsilber, J., et al., Anal Chem 75, 663-70 (2003).

### Mass Spectrometry

Early-eluting fractions were subsequently analyzed by reverse-phase LC-MS/MS using 75 μm inner diameter×12 cm self-packed fused-silica C18 capillary columns as described. Peptides were eluted for each analysis using a 6-hr gradient in which the ions were detected, isolated and fragmented in a completely automated fashion on an LCQ DECA XP ion trap mass spectrometer (Thermo Finnigan, San Jose, Calif.). In addition, software to allow for the acquisition of a data-dependent MS³ scan was produced and implemented through a collaboration with ThermoFinnigan. An MS³ spectrum was automatically collected when the most intense peak from the MS² spectrum corresponded to a neutral loss event of 98 m/z, 49 m/z.

### Database Correlation

All MS² and MS³ spectra were searched against the non-redundant human database from NCBI (downloaded Aug. 2003) using the Sequest algorithm. Eng, J., et al., J. Am. Soc. Mass Spectrom. 5, 976-989 (1994). Modifications were permitted to allow for the detection of oxidized methionine (+16), carboxyamidomethylated cysteine (+57), and phosphorylated serine, threonine and tyrosine (+80). All peptides matches were filtered and then manually validated with the aid of in-house software.

Classification And Bioinformatic Analysis Of Phosphorylation Sites

The ability of a protein kinase to carry out the phosphorylation reaction of a protein is highly related to the primary amino acid sequence surrounding the site of interest. Protein kinases can be separated into serine/threonine and tyrosine kinases, although dual specificity kinases exist. The sites detected from our nuclear preparation were entirely serine and threonine with no tyrosine phosphorylation detected. Tyrosine phosphorylation is generally thought to represent <1% of all cellular phosphorylation, but it is not clear what fraction of nuclear proteins are targets of tyrosine phosphorylation.

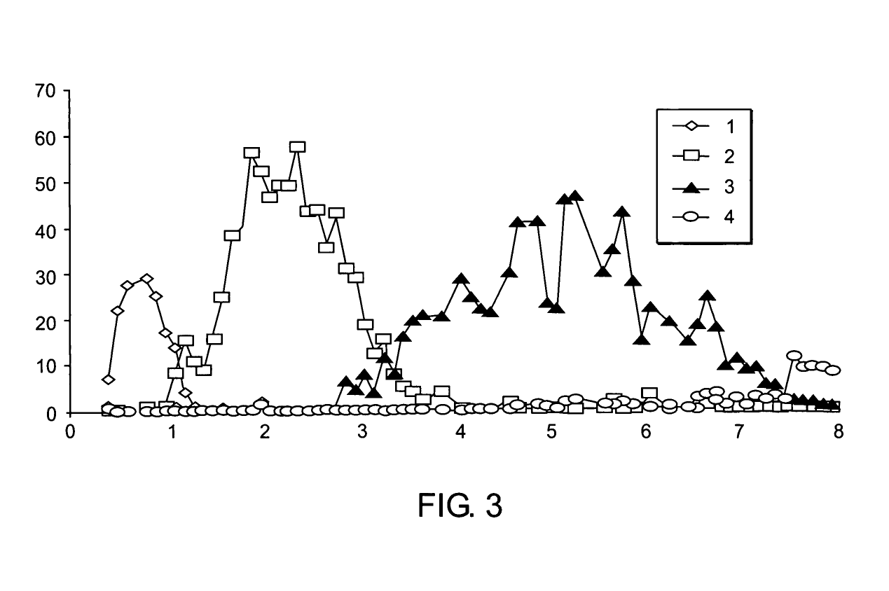

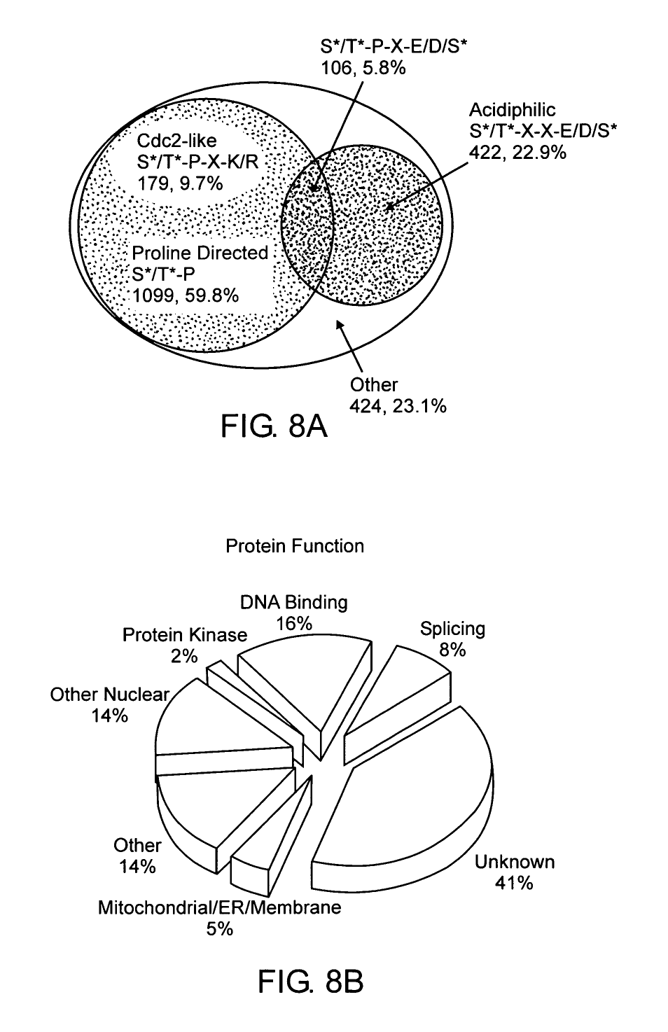

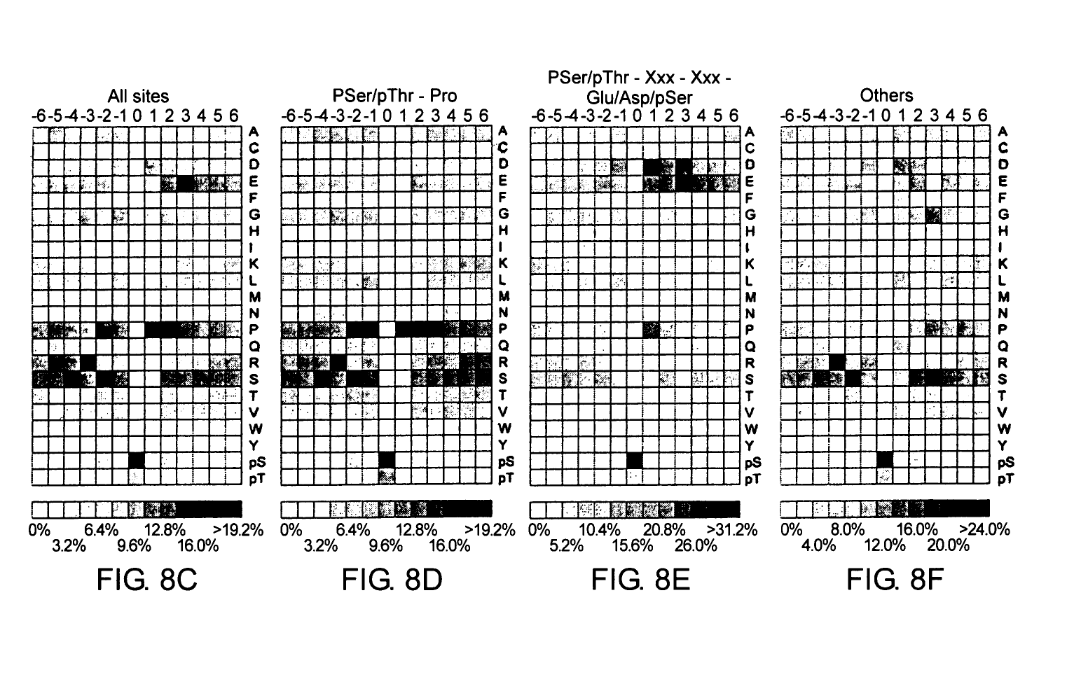

Serine/threonine protein kinases can be further subdivided based on substrate specificity which has been determined for a number of kinases by phosphorylation of soluble peptide libraries. Obenauer, J. C., et al., Nucleic Acids Res 31, 3635-41 (2003); O'Neill, T. et al., J Biol Chem 275, 22719-27 (2000). Major groups include proline-directed (e.g., Erk1, Cdk5, Cyclin B/Cdc2, etc.), basophilic (PKA, PKC, Slk1, etc.) and acidiphilic (CK 1 delta, CK 1 gamma, CK II) kinases. FIG. 3a shows that proline-directed and acidiphilic sites accounted for 77% of all detected phosphorylation. In addition, the sites detected can be categorized by their biological function (FIG. 8B). Consistent with our preparation, most sites detected were nuclear in origin or from other organelles known to be present in nuclear preparations (mitochondria, endoplasmic reticulum). Finally, numerous protein kinases and transcription factors were identified demonstrating the sensitivity of the analysis. Table 2 shows 62 phosphorylation sites from 28 protein kinases detected in this study. Only six of these sites had been described previously.

TABLE 2
Phosphorylation Sites Determined From Protein Kinases Detected In This Study.

| Protein Name | Gene name | Peptide⁴ |
|---|---|---|
| Cell division cycle 2-like 1 | AF067512¹ | EYGS*PLKAYT*PVVVTLWYR |
| Tousled-like kinase 1 | AF162666¹ | ISDYFEYQGGNGSS*PVR |
| Tousled-like kinase 2 | AF162667¹ | ISDYFEFAGGSAPGTS*PGR |
| PAS-kinase | AF387103¹ | GLSS*GWSSPLLPAPVCNPNK |
| Cell division cycle 2-like 5 | AJ297709¹ | GGDVS*PSPYSSSSWR |
| | | S*PS*PAGGGSSPYSR |
| | | S*PSYSR |
| | | SLS*PLGGR |
| Unknown protein kinase | AK001247¹ | EGDPVSLSTPLETEFGSPSELS*PR |
| | | LSPDPVAGSAVSQELREGDPVSL . . . SELS*PR |
| | | VFPEPTES*GDEGEELGLPLLSTR |
| Cdc2-related PITSLRE alpha 2-1 | E54024² | DLLSDLQDIS*DSER |
| Serine/threonine protein kinase | G01025² | VPAS*PLPGLER |
| Mitogen-and stress-activated protein kinase-1 | T13149² | LFQGYS*FVAPSILFK |
| Serine-protein kinase ATM | ATM_HUMAN³ | SLAFEEGS*QSTTISSLSEK |
| Cell division protein kinase 2 | CDK2_HUMAN³ | IGEGT*YGVVYK |
| Cell division cycle 2-related protein kinase 7 | CRK7_HUMAN³ | AIT*PPQQPYK |
| | | GS*PVFLPR |
| | | NSS*PAPPQPAPGK |
| | | QDDSPSGASYGQDYDLS*PSR |
| | | S*PGSTSR |
| | | SPS*PYSR |
| | | SVS*PYSR |
| | | TVDS*PK |
| Protein kinase C, delta type | KPCD_HUMAN³ | NLIDSMDQSAFAGFS*FVNPK |
| B-Raf proto-oncogene serine/threonine-protein kinase | RAB_HUMAN³ | GDGGSTTGLSAT*PPASLPGSLTNVK |
| | | SAS*EPSLNR |
| Megakaryocyte-associated tyrosine-protein kinase | MATK_HUMAN³ | SAGAPASVSGQDADGSTS*PR |
| Dual specificity mitogen-activated protein kinase kinase 2 | MPK2_HUMAN³ | LNQPGT*PTR |
| 3-phosphoinositide dependent protein kinase-1 | PDPK_HUMAN³ | ANS*FVGTAQYVSPELLTEK |
| Protein kinase C-like 1 | PKL1_HUMAN³ | TDVSNFDEEFTGEAPTLS*PPR |
| Protein kinase C-like 2 | PKL2_HUMAN³ | AS*SLGEIDESSELR |
| | | TST*FCGTPEFLAPEVLTETSYTR |
| Serine/threonine-protein kinase PRP4 homolog | PR4B_HUMAN³ | DAS*PINRWS*PTR |
| | | EQPEMEDANS*EKS*INEENGEVSEDQSQNK |
| | | S*LS*PKPR |
| | | S*PIINESR |
| | | S*PVDLR |
| | | S*RS*PLLNDR |
| | | SINEENGEVS*EDQS*QNK |
| | | TLS*PGR |
| | | TRS*PS*PDDILER |
| | | YLAEDSNMSVPSEPSS*PQSSTR |
| DNA-dependent protein kinase catalytic subunit | PRKD_HUMAN³ | LTPLPEDNS*MNVDQDGDPSDR |
| Serine/threonine protein kinase 10 | STKA_HUMAN³ | QVAEQGGDLS*PAANR |
| Wee1-like protein kinase | WEE1_HUMAN³ | SPAAPYFLGSSFS*PVR |
| Mitogen-activated protein kinase kinase kinase kinase 1 | M4K1_HUMAN³ | DLRS*SS*PR |
| Mitogen-activated protein kinase kinase kinase kinase 4 | M4K4_HUMAN³ | AASSLNLS*NGETESVK |
| | | TTS*RS*PVLSR |
| Mitogen-activated protein kinase kinase kinase kinase 6 | M4K6_HUMAN³ | LDSS*PVLSPGNK |
| Casein Kinase I, epsilon isoform | KC1E_HUMAN³ | IQPAGNTS*PR |
| Phosphorylase B kinase, beta regulatory chain | KPBB_HUMAN³ | QSST*PSAPELGQQPDVNISEWK |

¹Accession number derived from GenBank (NCBI).
²Accession number derived from the Protein Information Resource (PIR).
³Accession number derived from SwissProt human database.
⁴Site of phosphorylation noted by asterisk (*).

The computer algorithm, Scansite (Obenauer, J. C., et al., Nucleic Acids Res 31, 363541 (2003)), makes use of soluble peptide library phosphorylation data to create matrices useful for the prediction of a linear amino acid sequence as a substrate for recognition by a specific kinase. Table 3 shows the results of correlating the linear sequences surrounding the sites identified by this study against the known matrices at 10 the highest stringency level (0.002) and a lower stringency level (0.01).

TABLE 3
Scansite Prediction At Highest Stringency (0.2%) And Medium
Stringency (1.0%) For Kinase Phosphorylation And
Binding Motifs From This Dataset

| | | Hits | |
|---|---|---|---|
| Kinase | Type | (0.2%) | Hits (1.0%) |
| Casein Kinase 2 | Acidiphilic | 65 | 172 |
| GSK3 | Proline-directed | 64 | 206 |
| CDC2 | Proline-directed | 55 | 262 |
| AKT | Basophilic | 53 | 122 |
| Erk1 | Proline-directed | 51 | 235 |
| CDK5 | Proline-directed | 49 | 260 |
| P38 map kinase | Proline-directed | 33 | 160 |
| Protein Kinase A | Basophilic | 17 | 48 |
| Clk2 | Basophilic | 11 | 72 |
| DNA-PK | Glutamine-directed | 8 | 62 |
| Cam Kinase 2 | Basophilic | 7 | 21 |
| ATM | Glutamine-directed | 6 | 23 |
| PKC delta | Basophilic | 2 | 9 |
| PKC alpha/beta/gamma | Basophilic | 1 | 7 |
| Protein Kinase C epsilon | Basophilic | 1 | 8 |
| Casein Kinase 1 | Other | 0 | 23 |
| Protein Kinase D | Basophilic | 0 | 5 |
| 14-3-3 binding motif | Proline-directed | 31 | 85 |
| PDK1 binding motif | Proline-directed | 2 | 3 |

At the highest stringency, Scansite predicted a significant number of phosphorylation sites within our dataset from each of the proline-directed kinases, the basophilic kinases (AKT, PKA, and Clk2), the acidiphilic kinase Casein kinase 2, and the DNA damage activated kinases ATM and DNA-PK. It is also possible to use Scansite matrices to predict sites which require phosphorylation to become suitable binding domains. Our dataset included several known 14-3-3 binding sites, as well as two known PDK1 binding sites from protein kinase C delta and p90RSK. However, only a fraction of the total number of detected sites could be assigned with high confidence by Scansite suggesting that many more kinase motifs are present in our dataset.

With a dataset of this magnitude it is possible to begin to classify phosphorylation sites into specific motifs. To evaluate potential kinase motifs within such a large dataset, the relative occurrence of each amino acid (including pSer/pThr) flanking the site of phosphorylation was calculated and plotted using intensity maps. An examination of the entire dataset (FIG. 8C) revealed that a proline at the +1 position and/or a glutamic acid at position +3 were favored. To further elucidate significant flanking residues, the same maps were generating considering data which conformed to either pSer/pThr - Pro containing sites (FIG. 8D), pSer/pThr—Xxx—Xxx Glu/Asp/pSer containing sites (FIG. 8E), or the subset of all data which did not conform to either general classification (FIG. 8F).

Several further insights into kinase motifs can be made from the plots. For example, in FIG. 8E which shows the acidic residue at +3, it can be seen that an aspartic acid residue is highly favored at position +1 in this subset. Although this was not predicted by the soluble peptide libraries (Songyang, Z. et al., Mol Cell Biol 16, 6486-93 (1996)), a propensity for aspartic acid at the +1 position of Casein kinase 2 sites has been reported (Meggio, F., et al., Faseb J 17, 349-68 (2003)). In the proline-directed subset (FIG. 8D) additional prolines at the +2 and +3 position as well as serine at −3 and arginine at −2 are favored.

### Discussion

In eukaryotic cells, protein kinases add a phosphate moiety in an ATP-dependent manner to a serine, threonine, or tyrosine residue of a substrate protein. In addition to a critical role in normal cellular processes, malfunctions in protein phosphorylation have been implicated in the causation of many diseases such as diabetes, cancer, and Alzheimer's disease. With more than 500 members and thousands of potential substrates, human protein kinases remain attractive drug targets, yet the therapeutic promise of intervention in protein phosphorylation systems remains almost entirely unrealized.

The method described here exploits a differential solution state charge of most tryptic phosphopeptides when compared with their nonphosphorylated counterparts. Because SCX chromatography separates peptides primarily based-on charge, phosphopeptides containing a single basic group elute first and are highly enriched. The enriched phosphopeptides are then “sequenced” by reverse-phase LC-MS/MS with a new data-dependent acquisition of an MS³ scan whenever a phosphopeptide is suspected. In this way, large numbers of phosphopeptides can be isolated, separated, and sequence-analyzed in an automated fashion. The identification of 2,002 phosphorylation sites from a HeLa cell nuclear preparation is provided to demonstrate the technique. This is the largest dataset of post-translational modifications ever determined.

Multidimensional chromatography often plays a key role in proteome analysis strategies. SCX chromatography is the most common primary separation tool prior to analysis by reverse-phase LC-MS/MS. The strategy reported here utilized off-line SCX chromatography with fraction collection. Because tryptic phosphopeptides eluted early (FIG. 6C), it is unlikely that these peptides would be amenable to analysis by on-line SCX chromatography utilizing “salt bumps”.

This dataset provides new bioinformatic opportunities to study and predict kinase-substrate relationships. The intensity maps in FIG. 8 provide some insight into sequence specific trends surrounding each phosphorylation site. Proline-directed and acidiphilic kinases make up a large fraction of our dataset.

The SCX isolation method has the caveat that some sites are not amenable to analysis. Specifically, a histidine-containing phosphopeptide would elute as a 2+peptide. Similarly a doubly-phosphorylated tryptic peptide with only two basic sites would have a net charge state of zero. In essence, any phosphorylated peptide with a charge state other than 1⁺ would not be detected by the method as implemented in this example. Importantly, the majority of phosphopeptides are predicted to be amenable to isolation via SCX chromatography (FIG. 6B).

The methodology of this invention significantly enhances the ability to routinely discover large numbers of phosphorylated species within complex protein mixtures by exploiting peptide solution charge states generated by tryptic digests. Enrichment by offline SCX chromatography increases the likelihood of selecting phosphorylated peptides for sequencing in the mass spectrometer, while data-dependent MS³ software aids in confirming sequence and phosphorylation site location. Finally, the combination of stable isotope labeling with the methods described here would allow for a large-scale comparative phosphorylation analysis of different cell states where several hundred phosphorylation sites could be simultaneously profiled.

The methods of the present invention also are suitable for the identification of the N-terminal peptide of most proteins after trypsin digestion. This is because an acetylated N terminus will produce a peptide with a solution charge state of 1+ at pH 3 after trypsin digestion. These peptide are co-eluting with the phosphopeptides and can be detected in the same regions of the chromatogram. In the example below, the N-terminal peptide from more than 400 yeast proteins are sequenced. Because the N terminus is only acetylated about 50% of the time in vivo, the N termini were chemically modified by d3-acetylation. In this way, it can be determined i) whether or not the protein was present in a blocked (acetylated) state, and ii) whether or not the initiator methionine residue was cleaved. Tables 5A and 5B contain the list of proteins, their starting residues, and acetylation state.

### Example 3

### Determining N-terminal Sequences And N-terminal Modifications Of Proteins From Saccharomyces cerevisiae On A Large Scale

S. cerevisiae strain S288C was grown on YPD-medium (Becton and Dickinson) at 30° C. to midlog phase (OD₆₀₀ of 1). Approximately 3×10⁹ cells were harvested by centrifugation and the cell-pellet was resuspended in lysis buffer (50 mM Tris-HCl, pH 7.6, 0.1% SDS, 5 mM EDTA, and a protease inhibitor cocktail: 2 μg/ml aprotinin; 10 μg/ml leupeptin, soybean trypsin inhibitor, and pepstatin; 175 μg/ml phenylmethylsulfonyl fluoride) and lysed using a French press. About 1 mg proteins from the obtained yeast whole cell lysate were separated on a 12% SDS-PAGE gel. The gel was cut into 5 slices and the proteins were in-gel modified as described in the following: reduction with 10 mM DTT (pH 8.0) at 56° C., alkylation of Cys-residues with 55 mM iodoacetamide (pH 8.0) at RT in the dark, and d₃-acetylation of unblocked amino groups with 50 mM NH₄HCO₃ (pH 8.0)/MeOH/d₆-acetic anhydride (Sigma) 56:22:22 (v/v/v) at RT. Thevis, M. et al. (2003) J. Proteome Res. 2, 163-172.

The proteins were finally in-gel digested with modified trypsin (Promega), the peptides were extracted from the gel, and the peptides from each of the 5 gel slices were subjected individually to strong cation-exchange (SCX) chromatography on a 2.1×200 mm Polysulfoethyl A column (Poly LC) using a liquid phase from Buffer A (5 mM KH₂PO₄ pH 2.7, 33% ACN) and Buffer B (5 mM KH₂PO₄ pH 2.7, 33% ACN, 350 mM KCl). A gradient of 5 to 60% Buffer B in 50 min was applied and fractions were collected every 4 min. The fractions taken within the retention time range of 2 to 22 min were lyophilized, the residues were resuspended in H₂O/ACN/TFA 94.5:5:0.5 (v/v/v) and desalted using C18 solid-phase extraction (SPE) cartridges (BioSelect, Vydac).

The desalted samples were analyzed by reversed-phase nano-scale microcapillary high-performance liquid chromatography-tandem mass spectrometry (RP-LC-MS/MS) using a 150 μm×10 cm capillary column self-packed with C₁₈-bonded silica (Magic C₁₈ AQ, Michrom Bioresources), an Agilent 1100 binary pump (Buffer A, 2.5% ACN and 0.1% FA in water; Buffer B, 2.5% ACN and 0.1% FA in ACN; 60 min gradient from 5 to 35% Buffer B in 60 min; flow rate, 300 nl/min), a Famos autosampler (LC Packings), and an LTQ FT mass spectrometer (Thermo Electron). The mass spectra were obtained in an automated fashion by acquiring 1 FTICR-MS scan followed by 10 data-dependent LTQ-MS/MS scans in a cycle time of approximately 4 sec. MS/MS spectra were searched against the known yeast ORF database using the Sequest algorithm. Eng, J. et al. (1994) J. Am. Soc. Mass. Spectrom. 5, 976-989.

The Sequest results were filtered using in-house software. Minimum XCorr scores were set at 2, 2, and 3 for charge states 1+, 2+, and 3+, respectively. After searching using no enzyme specificity, only peptides that started with a Met or with a residue following a Met in the database entry, and ended with an Arg were considered for further manual validation. The resulting N-terminal peptides are listed in Table 5A and Table 5B.

Variations, modifications, and other implementations of what is described herein will occur to those of ordinary skill in the art without departing from the spirit and scope of the invention as described and claimed herein and such variations, modifications, and implementations are encompassed within the scope of the invention.

All of the references, patents and patent applications identified hereinabove are expressly incorporated herein by reference in their entireties.

TABLE 4
Hela Phosphorylation Peptides

| Peptide | Protein |
|---|---|
| SS*DGEEAEVDEER | GP: AB000516_1 |
| APS*LTDLVK | GP: AB002293_1 |
| LSGEGDTDLGALSNDGSDDGPSVMDETS*NDAFDSLER | GP: AB002293_1 |
| LVEPHS*PS*PSSK | GP: AB002293_1 |
| TNS*MGSATGPLPGTK | GP: AB002293_1 |
| TNS*PAYSDIS*DAGEDGEGKVDSVK | GP: AB002293_1 |
| GSVSQPST*PS*PPKPTGIFQTSANSSFEPVK | GP: AB002308_1 |
| VKS*PS*PK | GP: AB002330_1 |
| DTGSEVPSGSGHGPCT*PPPAPANFEDVAPTGSGEPGATR | GP: AB002337_1 |
| NGPLPIPSEGS*GFTK | GP: AB002366_1 |
| LIDLES*PTPESQK | GP: AB007900_1 |
| TLS*DESIYNSQR | GP: AB007900_1 |
| EASAS*PDPAK | GP: AB007947_1 |
| VPGPEEALVTQDQAWS*EAHAS*GEKR | GP: AB009265_1 |
| GGPEGVAAQAVASAASAGPADAEMEEIFDDAS*PGKQK | GP: AB010882_1 |
| RVS*PLNLSSVTP | GP: AB011472_1 |
| SQLQALHIGLDSSS*IGS*GPGDAEADDGFPESR | GP: AB014519_1 |
| GEQLRPWAPGDLS*VM | GP: AB014543_1 |
| KAS*VVDPSTESSPAPQEGSEQPASPAS*PLSSR | GP: AB014576_1 |
| TVFPGAVPVLPAS*PPPK | GP: AB015346_1 |
| AAFGIS*DSYVDGSSFDPQR | GP: AB016092_1 |
| AGMS*SNQSISSPVLDAVPR | GP: AB016092_1 |
| AGMSSNQSISS*PVLDAVPR | GP: AB016092_1 |
| APS*PSSR | GP: AB016092_1 |
| AQSGS*DSSPEPK | GP: AB016092_1 |
| AQSGSDSS*PEPK | GP: AB016092_1 |
| AQT*PPGPSLSGSK | GP: AB016092_1 |
| CLT*PQR | GP: AB016092_1 |
| DGSGT*PSR | GP: AB016092_1 |
| DQQSSS*SER | GP: AB016092_1 |
| ELSNS*PLR | GP: AB016092_1 |
| ENS*FGSPLEFR | GP: AB016092_1 |
| FQSDSSS*YPTVDSNSLLGQSR | GP: AB016092_1 |
| GEFSAS*PMLK | GP: AB016092_1 |
| LPQSSSSESSPPS*PQPTK | GP: AB016092_1 |
| MALPPQEDATAS*PPR | GP: AB016092_1 |
| MAPALSGANLTS*PR | GP: AB016092_1 |
| MGQAPSQSLLPPAQDQPRS*PVPSAFSDQSR | GP: AB016092_1 |
| QGSITS*PQANEQSVTPQR | GP: AB016092_1 |
| QGSITSPQANEQSVT*PQR | GP: AB016092_1 |
| QS*HSES*PSLQSK | GP: AB016092_1 |
| QS*HSGSIS*PYPK | GP: AB016092_1 |
| S*DTSSPEVR | GP: AB016092_1 |
| S*GAGSSPETK | GP: AB016092_1 |
| S*GMSPEQSRFQS*DSSSYPTVDSNSLLGQSR | GP: AB016092_1 |
| S*GSESSVDQK | GP: AB016092_1 |
| S*GSSPEVDSK | GP: AB016092_1 |
| S*GSSPGLR | GP: AB016092_1 |
| S*GTPPRQGS*ITSPQANEQSVTPQR | GP: AB016092_1 |
| S*PPAIR | GP: AB016092_1 |
| S*PSSPELNNK | GP: AB016092_1 |
| S*PVPSAFSDQSR | GP: AB016092_1 |
| S*RS*PLAIR | GP: AB016092_1 |
| S*RT*PPSAPSQSR | GP: AB016092_1 |
| S*SPELTR | GP: AB016092_1 |
| S*TSADSASSSDTSR | GP: AB016092_1 |
| S*TTPAPK | GP: AB016092_1 |
| S*VSPCSNVESR | GP: AB016092_1 |
| SAT*PPATR | GP: AB016092_1 |
| SATRPS*PS*PER | GP: AB016092_1 |
| SDTSS*PEVR | GP: AB016092_1 |
| SECDSS*PEPK | GP: AB016092_1 |
| SES*DSSPDSK | GP: AB016092_1 |
| SGAGSS*PETK | GP: AB016092_1 |
| SGMS*PEQSR | GP: AB016092_1 |
| SGS*ESSVDQK | GP: AB016092_1 |
| SGS*SPEVDSK | GP: AB016092_1 |
| SGS*SPEVK | GP: AB016092_1 |
| SGS*SPGLR | GP: AB016092_1 |
| SGSESS*VDQK | GP: AB016092_1 |
| SGSS*PEVDSK | GP: AB016092_1 |
| SGSS*PEVK | GP: AB016092_1 |
| SGSS*PGLR | GP: AB016092_1 |
| SGT*PPRQGSITS*PQANEQSVTPQR | GP: AB016092_1 |
| SLS*YSPVER | GP: AB016092_1 |
| SLSYS*PVER | GP: AB016092_1 |
| SPS*PASGR | GP: AB016092_1 |
| SPS*SPELNNK | GP: AB016092_1 |
| SPSS*PELNNK | GP: AB016092_1 |
| SRS*GSS*PEVDSK | GP: AB016092_1 |
| SRS*PSS*PELNNK | GP: AB016092_1 |
| SRS*TT*PAPK | GP: AB016092_1 |
| SRT*S*PVTR | GP: AB016092_1 |
| SS*PELTR | GP: AB016092_1 |
| SS*PEPK | GP: AB016092_1 |
| SS*TPPRQS*PSR | GP: AB016092_1 |
| SSS*ASSPEMK | GP: AB016092_1 |
| SSS*PQPK | GP: AB016092_1 |
| SSS*PVTELASR | GP: AB016092_1 |
| SSS*PVTELASRS*PIR | GP: AB016092_1 |
| SSSAS*SPEMK | GP: AB016092_1 |
| SSSS*PPPK | GP: AB016092_1 |
| SST*PPGESYFGVSSLQLK | GP: AB016092_1 |
| SST*PPRQS*PSR | GP: AB016092_1 |
| SSTGPEPPAPT*PLLAER | GP: AB016092_1 |
| ST*TPAPK | GP: AB016092_1 |
| STSADSASSSDT*SR | GP: AB016092_1 |
| STT*PAPK | GP: AB016092_1 |
| T*PLISR | GP: AB016092_1 |
| T*PPVALNSSR | GP: AB016092_1 |
| T*PPVTR | GP: AB016092_1 |
| TPAAAAAMNLAS*PR | GP: AB016092_1 |
| TPQAPAS*ANLVGPR | GP: AB016092_1 |
| TS*PPLLDR | GP: AB016092_1 |
| VKS*SP*PPR | GP: AB016092_1 |
| VPS*PTPAPK | GP: AB016092_1 |
| YSHSGSS*S*PDTK | GP: AB016092_1 |
| ETESAPGS*PR | GP: AB018274_1 |
| ST*PSLER | GP: AB018306_1 |
| AITSLLGGGS*PK | GP: AB019494_1 |
| NNTAAETEDDES*DGEDR | GP: AB019494_1 |
| GPSQATS*PIR | GP: AB020626_1 |
| EPVS*PMELTGPEDGAASSGAGR | GP: AB020683_1 |
| S*PLSWK | GP: AB020683_1 |
| ANS*QENR | GP: AB020689_1 |
| T*PTMPQEEAAEK | GP: AB020711_1 |
| STGS*ATSLASQGER | GP: AB022657_1 |
| STGSATSLAS*QGER | GP: AB022657_1 |
| RPASPPAGLALAPRS*PSAS*PEPREGETLS*PSMQR | GP: AB023163_1 |
| TNAVS*PK | GP: AB023227_1 |
| ST*SIHYADSVK | GP: AB027443_1 |
| YSVGSLS*PVSASVLK | GP: AB028069_1 |
| SATTTPSGS*PR | GP: AB028971_1 |
| SKS*ATTTPS*GSPR | GP: AB028971_1 |
| VQTT*PPPAVQGQK | GP: AB028971_1 |
| AAKPGPAEAPS*PTASPSGDAS*PPATAPYDPR | GP: AB028987_1 |
| TGSGS*PFAGNSPAR | GP: AB028987_1 |
| TGSGSPFAGNS*PAR | GP: AB028987_1 |
| SNGELSES*PGAGK | GP: AB032251_1 |
| IVPQSQVPNPES*PGK | GP: AB033023_1 |
| IVSGS*PISTPSPSPLPR | GP: AB033023_1 |
| QPGQVIGATTPSTGS*PTNK | GP: AB033023_1 |
| AGSSAAGASGWTSAGSLNSVPTNSAQQGHNS*PDS*PVTSAAK | GP: AB036090_1 |
| ANFDEENAYFEDEEEDSSNVDLPYIPAENS*PTR | GP: AB036090_1 |
| APDMSS*SEEFPSFGAQVAPK | GP: AB036090_1 |
| NS*PSAASTSSNDSK | GP: AB036737_1 |
| S*PTPALCDPPACSLPVASQPPQHLSEAGR | GP: AB036737_1 |
| VAS*DTEEADR | GP: AB036737_1 |
| ASDPQS*PPQVSR | GP: AB037782_1 |
| QVPHSS*R | GP: AB037813_1 |
| EFLPTSWS*PVGAGPTPSLYK | GP: AB037824_1 |
| SLDSEPSVPSAAKPPS*PEK | GP: AB037911_1 |
| GSS*PEAGAAAMAESIIIR | GP: AB040932_1 |
| DQS*PPPS*PPPSYHPPPPPTK | GP: AB040955_1 |
| GLAGPPAS*PGK | GP: AB040955_1 |
| GS*PSGGSTAEASDTLSIR | GP: AB040955_1 |
| S*PGASVSSSLTSLCSSSSDPAPSDR | GP: AB040955_1 |
| TLS*PSSGYSSQSGTPTLPPK | GP: AB040955_1 |
| EAS*PAPLAQGEPGR | GP: AB040975_1 |
| SEVYDPSDPTGSDSSAPGSS*PER | GP: AB040975_1 |
| GTEAS*PPQNNSGSSSPVFTFR | GP: AB040976_1 |
| S*PGPGPSQSPR | GP: AB040976_1 |
| YLLGNAPVS*PSSQK | GP: AB041557_1 |
| NALTTLAGPLT*PPVK | GP: AB044549_1 |
| SPTAPSVFS*PTGNR | GP: AB044549_1 |
| LQQTVPADAS*PDSK | GP: AB045733_1 |
| GPVGVCS*YTPTPVGRTMSLVSQNS*R | GP: AB046807_1 |
| APS*PPPTASNSSNSQ | GP: AB046830_1 |
| APSPPPTAS*NSSNSQ | GP: AB040830_1 |
| DCSYGAVTS*PTSTLESR | GP: AB046856_1 |
| LSS*LSSQTEPTSAGDQYDCSR | GP: AB051458_1 |
| LTQAEISEQPTMATVVPQVPTS*PK | GP: AB051468_1 |
| APS*PTGPALISGAS*PVHCAADGTVELK | GP: AB051472_1 |
| FQAPS*PSTLLR | GP: AB051485_1 |
| NSSLGSPSNLCGS*PPGSIR | GP: AB051540_1 |
| RAS*QSS*LESSTGPPCIR | GP: AB051866_1 |
| AFLASLS*PAMVVPEDQLTR | GP: AB053172_1 |
| NEEPIDSEQDENIDT*R | GP: AB055056_1 |
| SPS*PVQGK | GP: AB056107_1 |
| GPS*PPGAK | GP: AB056152_1 |
| S*PSVS*PSKQPVSTSSK | GP: AB058764_1 |
| EVS*PSDVR | GP: AB059277_1 |
| S*TPRSTPLASPSPS*PGR | GP: AB059277_1 |
| LSLS*PLR | GP: AB062430_1 |
| T*PS*PESHR | GP: AB062430_1 |
| GS*PQPQQEPR | GP: AB063357_1 |
| T*VPLPPS*SAM | GP: AB067519_1 |
| AES*PEEVACR | GP: AB071605_1 |
| AGSST*PGDAPPAVAEVQGR | GP: AB071605_1 |
| DGGS*GNSTIIVSR | GP: AB071605_1 |
| GSGTAS*DDEFENLR | GP: AB071605_1 |
| SDGSGESAQPPEDSS*PPASSESSSTR | GP: AB071605_1 |
| S*PSWMSK | GP: AB072355_1 |
| QQEEEAVELQPPPPAPLS*PPPPAPTAPQPPGDPLMSR | GP: AB075829_1 |
| QTSYEAS*PR | GP: AB082522_1 |
| SQS*CSDTAQER | GP: AB082522_1 |
| VLDTSSLTQSAPAS*PTNK | GP: AB082951_1 |
| QT*VPTPVR | GP: AB086011_1 |
| LSVPT*S*DEEDEVPAPKPR | GP: AB088096_1 |
| AQPFGFIDS*DTDAEEER | GP: AB088099_1 |
| DSDT*DVEEEELPVENR | GP: AB088099_1 |
| GQASS*PTPEPGVGAGDLPGPTSAPVPSGS*QSGGRGSPVSPR | GP: AB088099_1 |
| GQASS*PTPEPGVGAGDLPGPTSAPVPSGSQSGGRGS*PVSPR | GP: AB088099_1 |
| LEPSTSTDQPVT*PEPTSQATR | GP: AB088099_1 |
| LLLAEDS*EEEVDFLSER | GP: AB088099_1 |
| SQTTTERDS*DT*DVEEEELPVENR | GP: AB088099_1 |
| SSVKT*PETVVPTAPELQPSTSTDQPVTPEPTSQATR | GP: AB088099_1 |
| TPETVVPTAPELQPSTST*DQPVTPEPTSQATR | GP: AB088099_1 |
| LGYLVS*PPQQIR | GP: AB112075_1 |
| S*PPYPR | GP: AB112075_1 |
| S*PQAFR | GP: AB112075_1 |
| VTGTEGSSSTLVDYTSTSSTGGS*PVR | GP: AB112075_1 |
| MEEEGTEDNGLEDDS*R | GP: AC004611_1 |
| NTLETSS*LNFK | GP: AC004611_1 |
| VTPDIEES*LLEPENEK | GP: AC004611_1 |
| LGASNS*PGQPNSVK | GP: AC004858_3 |
| FAELPEFRPEEVLPSPT*LQSLATS*PR | GP: AC006486_3 |
| NSCQDS*EADEETSPGFDEQEDGSSSQTANKPSR | GP: AF005043_1 |
| GVS*MPNMLEPK | GP: AF005654_1 |
| STS*QGSINSPVYSR | GP: AF005654_1 |
| TAS*LPGYGR | GP: AF005654_1 |
| TLS*PTPSAEGYQDVR | GP: AF005654_1 |
| QEQINTEPLEDTVLS*PTK | GP: AF017633_1 |
| EVDGLLTSEPMGS*PVSSK | GP: AF034373_1 |
| GPPQS*PVFEGVYNNSR | GP: AF034373_1 |
| LQPSSS*PENSLDPFPPR | GP: AF034373_1 |
| AWGPGLHGGIVGRS*ADFVVESIGSEVGSLGFAIEGPSQAK | GP: AF042166_1 |
| SETDLSS*LTASIK | GP: AF042166_1 |
| SRSQSPS*PS*PAR | GP: AF042800_1 |
| TSSGAGSPAVAVPTHSQPSPT*PS*NESTDTASEIGSAFNSPLR | GP: AF045581_1 |
| S*FDYNYR | GP: AF047448_1 |
| AAS*PS*PQSVRR | GP: AF048977_1 |
| APQTSSS*PPPVR | GP: AF048977_1 |
| GTS*AEQDNR | GP: AF048977_1 |
| KAAS*PS*PQSVR | GP: AF048977_1 |
| KPPAPPS*PVQSQS*PSTNWSPAVPVK | GP: AF048977_1 |
| KPPAPPS*PVQSQSPSTNWS*PAVPVK | GP: AF048977_1 |
| LSPSAS*PPR | GP: AF048977_1 |
| MAAADS*VQQR | GP: AF048977_1 |
| QNQQSSSDSGSSS*SS*EDERPK | GP: AF048977_1 |
| RAS*PS*PPPK | GP: AF048977_1 |
| RLS*PSAS*PPR | GP: AF048977_1 |
| RLSPS*AS*PPR | GP: AF048977_1 |
| RS*PS*PAPPPR | GP: AF048977_1 |
| RT*PS*PPPR | GP: AF048977_1 |
| RYS*PS*PPPK | GP: AF048977_1 |
| S*PQPNK | GP: AF048977_1 |
| S*PS*PPPTRR | GP: AF048977_1 |
| S*PSPAPPPR | GP: AF048977_1 |
| S*PSPPPTR | GP: AF048977_1 |
| SASPS*PR | GP: AF048977_1 |
| SPS*PAPEK | GP: AF048977_1 |
| SPS*PAPPPR | GP: AF048977_1 |
| SPS*PPPTR | GP: AF048977_1 |
| SRVS*VS*PGR | GP: AF048977_1 |
| SVS*GSPEPAAK | GP: AF048977_1 |
| SVSGS*PEPAAK | GP: AF048977_1 |
| T*AS*PPPPPKR | GP: AF048977_1 |
| T*PELPEPSVK | GP: AF048977_1 |
| T*PT*PPPRR | GP: AF048977_1 |
| T*PTPPPR | GP: AF048977_1 |
| TAS*PPPPPK | GP: AF048977_1 |
| TPS*PPPR | GP: AF048977_1 |
| VSVS*PGRT*SGK | GP: AF048977_1 |
| YSPS*PPPK | GP: AF048977_1 |
| SFTSSSPSS*PSR | GP: AF049884_1 |
| YQT*QPVTLGEVEQVQSGK | GP: AF051850_1 |
| AGNALT*PELAPVQIK | GP: AF052052_1 |
| KGS*DDDGGDS*PVQDIDTPEVDLYQLQVNTLR | GP: AF055993_1 |
| LFDVCGS*QDFESDLDR | GP: AF057299_1 |
| VFQT*EAELQEVISDLQSK | GP: AF057299_1 |
| TTTPGPSLS*QGVSVDEK | GP: AF058696_1 |
| TIS*PPTLGTLR | GP: AF060479_1 |
| AYT*PVVVTLWYR | GP: AF067512_1 |
| EYGS*PLKAYT*PVVVTLWYR | GP: AF067512_1 |
| AES*PGPGSR | GP: AF075587_1 |
| GLS*VDSAQEVK | GP: AF076974_1 |
| KPVTVSPTTPTS*PTEGEAS | GP: AF078849_1 |
| LGSTAPQVLSTSS*PAQQAENEAK | GP: AF078856_1 |
| ENS*PAAFPDR | GP: AF081287_1 |
| EAASS*PAGEPLR | GP: AF083106_1 |
| S*PGEPGGAAPER | GP: AF083106_1 |
| YMAENPTAGVVQEEEEDNLEYDS*DGNPIAPTK | GP: AF083255_1 |
| AILGSYDSELTPAEYS*PQLTR | GP: AF083811_1 |
| DIS*PEKSELDLGEPGPPGVEPPPQLLDIQCK | GP: AF090114_1 |
| FGQDIIS*PLLSVK | GP: AF092139_1 |
| ETEEQDS*DSAEQGDPAGEGK | GP: AF096870_1 |
| GGAPDPSPGATATPGAPAQPSS*PDAR | GP: AF097916_1 |
| VRGGAPDPSPGAT*ATPGAPAQPSS*PDAR | GP: AF097916_1 |
| QLLDS*DEEQEEDEGR | GP: AF098162_1 |
| RT*VAAPS*KR | GP: AF103483_1 |
| S*VTPPPPPR | GP: AF104413_1 |
| AALGLQDS*DDEDAAVDIDEQIESMFNSK | GP: AF106680_1 |
| ICS*DEEEDEEK | GP: AF108459_1 |
| QQDS*QPEEVMDVLEMVENVK | GP: AF112222_1 |
| TFS*ATVR | GP: AF115345_1 |
| EDYFEPIS*PDR | GP: AF116724_1 |
| DGEQS*PNVSLMQR | GP: AF116725_1 |
| DSALQDTDDS*DDDPVLIPGAR | GP: AF116725_1 |
| MEVGPFSTGQES*PTAENAR | GP: AF116730_1 |
| QGS*PVAAGAPAK | GP: AF117106_1 |
| EEQEILS*TR | GP: AF119230_1 |
| IPS*PNILK | GP: AF121141_1 |
| NKSSS*PEDPGAEV | GP: AF125568_1 |
| LGAGGGS*PEKS*PSAQELK | GP: AF129085_1 |
| LQVPTS*QVR | GP: AF133820_1 |
| SDDES*PSTSSGSSDADQRDPAAPEPEEQEER | GP: AF136176_1 |
| ILLVDS*PGMGNADDEQQEEGTSSK | GP: AF142328_1 |
| EIPSATQS*PISK | GP: AF147709_1 |
| DSGNWDTSGSELS*EGELEK | GP: AF151059_1 |
| SDSPES*DAER | GP: AF151059_1 |
| DWDKESDGPDDSRPESASDS*DT | GP: AF151873_1 |
| GESAPTLSTSPSPSSPSPTSPS*PTLGR | GP: AF153415_1 |
| WLDES*DAEMELR | GP: AF161470_1 |
| SEGEGEAASADDGSLNTS*GAGPK | GP: AF161491_1 |
| S*RIPSPLQPEMQGTPDDEPSEPEPS*PSTLIYR | GP: AF162447_1 |
| ISDYFEYQGGNGSS*PVR | GP: AF162666_1 |
| ISDYFEFAGGSAPGTS*PGR | GP: AF162667_1 |
| QLS*LEGS*GLGVEDLKDNTPSGK | GP: AF169548_1 |
| TYS*QDCSFK | GP: AF177387_1 |
| GGNLPPVS*PNDSGAK | GP: AF180425_1 |
| S*PEDQLGK | GP: AF180425_1 |
| STDSEVSQS*PAK | GP: AF180474_1 |
| GLNPDGTPALSTLGGFSPAS*KPSS*PR | GP: AF180920_1 |
| LS*PTPSMQDGLDLPSETDLR | GP: AF180920_1 |
| SPIS*INVK | GP: AF180920_1 |
| EAYSGCSGPVDSECPPPPS*SPVHK | GP: AF188700_1 |
| SGTSSPQS*PVFR | GP: AF188700_1 |
| TGS*NAAQYK | GP: AF188700_1 |
| QAEFFLS*QQASLLK | GP: AF191339_1 |
| RSS*FSMEEES | GP: AF196779_1 |
| AVGMPSPVS*PKLSPGNS*GNYSSGASSASASGSSVTIPQK | GP: AF197927_1 |
| LS*PGNSGNYSSGASSASASGSSVTIPQK | GP: AF197927_1 |
| NSYNNSQAPS*PGLGSK | GP: AF197927_1 |
| HGGS*PQPLATTPLSQEPVNPPSEAS*PTR | GP: AF201422_1 |
| HGGSPQPLATT*PLSQEPVNPPSEAS*PTR | GP: AF201422_1 |
| HGGSPQPLATTPLS*QEPVNPPSEASPT*R | GP: AF201422_1 |
| S*LSGSSPCPK | GP: AF201422_1 |
| S*PSVSSPEPAEK | GP: AF201422_1 |
| SASSS*PETR | GP: AF201422_1 |
| SHS*GSSSPS*PSR | GP: AF201422_1 |
| SLS*GSSPCPK | GP: AF201422_1 |
| SLSGS*SPCPK | GP: AF201422_1 |
| SLSGSS*PCPK | GP: AF201422_1 |
| SNS*SPEMK | GP: AF201422_1 |
| SNSS*PEMK | GP: AF201422_1 |
| SPS*VSSPEPAEK | GP: AF201422_1 |
| SPSVS*SPEPAEK | GP: AF201422_1 |
| SRS*VS*PCSNVESR | GP: AF201422_1 |
| SRT*PPTS*R | GP: AF201422_1 |
| SVS*PCSNVESR | GP: AF201422_1 |
| LEPQELS*PLSATVFPK | GP: AF203474_1 |
| ATGDGSS*PELPSLER | GP: AF205632_1 |
| SLS*ESSVIMDR | GP: AF205632_1 |
| KAEFPSSGSNSVLNT*PPTTPES*PSSVTVTEGSR | GP: AF214114_1 |
| DGGPVTS*QESGQK | GP: AF230336_1 |
| S*ESPSLTQER | GP: AF230336_1 |
| SES*PSLTQER | GP: AF230336_1 |
| SQNSQESTADES*EDDMSSQASK | GP: AF230336_1 |
| MS*VTGGK | GP: AF230929_1 |
| ALS*PAELR | GP: AF240677_1 |
| LAEAPSPAPTPSPTPVEDLGPQTSTSPGRLS*PDFAEELR | GP: AF240677_1 |
| AEGEPQEES*PLK | GP: AF249273_1 |
| FNDS*EGDDTEETEDYR | GP: AF249273_1 |
| IDIS*PSTLR | GP: AF249273_1 |
| S*GSGSVGNGSSR | GP: AF249273_1 |
| S*VSSQR | GP: AF249273_1 |
| SGS*GSVGNGSSR | GP: AF249273_1 |
| SGSGSVGNGS*SR | GP: AF249273_1 |
| SSATSGDIWPGLS*AYDNSPR | GP: AF249273_1 |
| SSATSGDIWPGLSAYDNS*PR | GP: AF249273_1 |
| SSS*PYSKS*PVSK | GP: AF249273_1 |
| SSSPYS*KS*PVSK | GP: AF249273_1 |
| SSSSSASPSS*PSSR | GP: AF249273_1 |
| SLS*VPVDLSR | GP: AF251040_1 |
| TVNSGGSSEPS*PTEVDVSR | GP: AF251055_1 |
| AAPPPPALT*PDSQTVDSSCK | GP: AF254411_1 |
| GPSPAPASS*PK | GP: AF254411_1 |
| QRS*PS*PAPAPAPAAAAGPPTR | GP: AF254411_1 |
| VPST*PPPK | GP: AF254411_1 |
| FADQDDIGNVS*FDR | GP: AF264779_1 |
| IQQFDDGGS*DEEDIWEEK | GP: AF264779_1 |
| ALVVPEPEPDSDS*NQER | GP: AF265230_1 |
| VDEDSAEDTQS*NDGK | GP: AF273048_1 |
| SCSPS*PVSPQVQPQAADTISDSVAVPASLLGMR | GP: AF273437_1 |
| TPIS*PLK | GP: AF273437_1 |
| TQS*LPVTEK | GP: AF273437_1 |
| STEDLS*PQK | GP: AF276423_1 |
| ESLPPAAEPS*PVSK | GP: AF283303_1 |
| GIGLDESELDS*EAELMR | GP: AF286340_1 |
| AAVGQES*PGGLEAGNAK | GP: AF294791_1 |
| EQSSEAAETGVS*ENEENPVR | GP: AF294791_1 |
| IISVT*PVK | GP: AF294791_1 |
| AQPGS*PESSGQPK | GP: AF297872_1 |
| LENEGS*DEDIETDVLYSPQMALK | GP: AF307332_1 |
| ATVPVAAATAAEGEGS*PPAVAAVAGPPAAAEVGGGVGGSSR | GP: AF308285_1 |
| S*PSPVQGK | GP: AF310246_1 |
| GSESSDT*DDEELR | GP: AF314184_1 |
| S*PIALPVK | GP: AF314184_1 |
| S*PS*PVPQEEHS*DPEMTEEEKEYQMMLLTK | GP: AF314184_1 |
| QAS*PTEVVER | GP: AF315591_1 |
| DGSS*PPLLEK | GP: AF317391_1 |
| LPEEDAS*SQSSK | GP: AF319995_1 |
| LSSSGAPPADFPS*PR | GP: AF319995_1 |
| TCGVNDDES*PSK | GP: AF319995_1 |
| WQLSS*PDGVDTDDDLPK | GP: AF319995_1 |
| T*DELNK | GP: AF322916_1 |
| MNGVMFPGNS*PSYTER | GP: AF327345_1 |
| NHSDSSTSESEVSSVS*PLK | GP: AF327345_1 |
| AGPSAQEPGSQT*PLK | GP: AF327452_1 |
| SAS*QSS*LDKLDQELK | GP: AF327452_1 |
| ATLSSTSGLDLMSESGEGEIS*PQR | GP: AF330045_1 |
| EVAATEEDVTRLPSPT*SPFS*SLSQDQAATSK | GP: AF330045_1 |
| ISINQT*PGK | GP: AF330045_1 |
| LPS*PTSPFSSLSQDQAATSK | GP: AF330045_1 |
| LPSPTS*PFSSLSQDQAATSK | GP: AF330045_1 |
| TPNNVVSTPAPS*PDASQLASSLSSQK | GP: AF330045_1 |
| VSAS*LPR | GP: AF330045_1 |
| VTTEIQLPSQS*PVEEQSPASLSSLR | GP: AF330045_1 |
| GS*PEPSALPPQR | GP: AF334584_1 |
| SAS*DSGCDPASK | GP: AF338242_1 |
| ATEDGEEDEVS*AGEK | GP: AF340183_1 |
| ADQGDGPEGS*GR | GP: AF349313_1 |
| DLNES*PVK | GP: AF349313_1 |
| VPS*PGMEEAGCSR | GP: AF349313_1 |
| ESGVVAVS*PEK | GP: AF356524_1 |
| NVDAAVS*PR | GP: AF356524_1 |
| RPQS*PGAS*PSQAER | GP: AF356524_1 |
| TGGS*PSVR | GP: AF356524_1 |
| ATPELGSSENSASS*PPR | GP: AF360549_1 |
| AQS*VSPVQAPPPGGSAQLLPGK | GP: AF363689_1 |
| KNS*TDLDSAPEDPTS*PK | GP: AF363689_1 |
| EGNTTEDDFPSS*PGNGNK | GP: AF374416_1 |
| SLS*NPDIASETLTLLS*FLR | GP: AF378754_1 |
| FPGDQVVNGAGPELSTGPSPGS*PTLDIDQSIEQLNR | GP: AF378756_1 |
| DPS*PESNK | GP: AF380154_1 |
| MDRT*PPPPTLS*PAAITVGR | GP: AF380154_1 |
| GLSS*GWSSPLLPAPVCNPNK | GP: AF387103_1 |
| GRLT*PS*PDIIVLSDNEASSPR | GP: AF411836_1 |
| GRLT*PSPDIIVLS*DNEASSPR | GP: AF411836_1 |
| LTPSPDIIVLSDNEASS*PR | GP: AF411836_1 |
| SAS*ADNLTLPR | GP: AF413522_1 |
| VPAEDETQSIDS*EDSFVPGR | GP: AF434816_1 |
| SDES*STEETDK | GP: AF441770_1 |
| SES*PCESPYPNEK | GP: AF441770_1 |
| TPATT*PEAR | GP: AF441770_1 |
| LASVLLYSDYGIGEVPVEPLDVPLPSTIRPAS*PVAGSPK | GP: AF453478_1 |
| AET*PPLPIPPPPPDIQPLER | GP: AF463523_1 |
| KPS*PAQAAETPALELPLPSVPAPAPL | GP: AF464935_1 |
| SKENGAS*V | GP: AF465616_1 |
| VEEESTGDPFGFDS*DDESLPVSSK | GP: AF479418_1 |
| GSEGSQS*PGSSVDDAEDDPSR | GP: AF488691_1 |
| SDS*DSSTLSK | GP: AF506799_1 |
| LQLS*DEESVFEEALMSPDTR | GP: AF506820_1 |
| APSPPPT*ASNSSNSQSEKEDGTVSTANQNGVSSNGPGEILNK | GP: AF515446_1 |
| YFDTNSEVEEES*EEDEDYIPSEDWK | GP: AF515446_1 |
| DSS*GQEDETQSSN | GP: AF515447_1 |
| NTPS*PDVTLGTNPGTEDIQFPIQK | GP: AF518874_1 |
| T*PVPTVSLASR | GP: AF520569_1 |
| S*AFPSFLVSFILF | GP: AF523356_1 |
| ATS*LTLEGGR | GP: AF533230_1 |
| QSSVTQVTEQS*PK | GP: AF534078_1 |
| AGSNEDPILAPSGT*PPPTIPPDETFGGR | GP: AF547989_1 |
| LEAAYS*PR | GP: AJ006778_1 |
| SLSDNGQPGT*PDPADSGGTSAK | GP: AJ006778_1 |
| IDGATQSS*PAEPK | GP: AJ223075_1 |
| TEVPGS*PAGTEGNCQEATGPSTVDTQNEPLDMK | GP: AJ223075_1 |
| DPGGITAGS*TDEPPMLTK | GP: AJ223980_1 |
| GTEPSPGGT*PQPSRPVS*PAGPPEGVPEEAQPPR | GP: AJ223980_1 |
| QEIES*DSESDGELQDRK | GP: AJ238403_1 |
| SCDELSPVS*PTQGGYPSEPTR | GP: AJ278120_1 |
| NFDFEGSLS*PVIAPK | GP: AJ278357_1 |
| SLCLS*PSEASQMK | GP: AJ278357_1 |
| EPDPFEFS*SGSESEGDIFTSPK | GP: AJ292190_1 |
| IPPMLS*PVHVQDSTDLAPPS*PEPPMLAPVAK | GP: AJ292190_1 |
| IPPMLSPVHVQDS*TDLAPPS*PEPPMLAPVAK | GP: AJ292190_1 |
| TAQSPAMVGS*PIR | GP: AJ292190_1 |
| WIPLSSDAQAPLAQPES*PTASAGDEPR | GP: AJ293573_1 |
| GGDVS*PSPYSSSSWR | GP: AJ297709_1 |
| HSSIS*PST*LTLK | GP: AJ297709_1 |
| S*PSPAGGGSSPYSR | GP: AJ297709_1 |
| S*PSYSR | GP: AJ297709_1 |
| SLS*PLGGR | GP: AJ297709_1 |
| SPS*PAGGGSSPYSR | GP: AJ297709_1 |
| FSGSKS*ANTAS*LTISGLR | GP: AJ399983_1 |
| CSDNSS*YEEPLSPISASSSTSR | GP: AJ419231_1 |
| ESCSS*PSTVGSSLTTR | GP: AJ430203_1 |
| LTSPVTSIS*PIQASEK | GP: AJ430203_1 |
| TITVPVSGS*PK | GP: AJ430203_1 |
| TNS*SSSSPVVLK | GP: AJ430203_1 |
| AVPMAPAPAS*PGSSNDSSAR | GP: AJ440784_1 |
| TLS*NESEESVK | GP: AJ459424_1 |
| TPTGS*PATEVSAK | GP: AJ459424_1 |
| DGQDAIAQS*PEK | GP: AK000867_1 |
| DSGS*DGEDDVNEQHSGS*DTGSVER | GP: AK000868_1 |
| S*QSIEQESQEK | GP: AK001192_1 |
| EGDPVSLSTPLETEFGSPSELS*PR | GP: AK001247_1 |
| LSPDPVAGSAVSQELREGDPVSLSTPLETEFGSPSELS*PR | GP: AK001247_1 |
| VFPEPTES*GDEGEELGLPLLSTR | GP: AK001247_1 |
| VTS*PTTYVLDEDEPR | GP: AK001544_1 |
| AVAS*PEATVSQTDENK | GP: AK001686_1 |
| ALSSGGSITS*PPLSPALPK | GP: AK001739_1 |
| KASS*PS*PLTIGTPESQR | GP: AK001969_1 |
| TSDDGGDS*PEHDTDIPEVDLFQLQVNTLR | GP: AK021588_1 |
| TGS*PTFVR | GP: AK021696_1 |
| SILPYPVS*PK | GP: AK022696_1 |
| DAEPQPGS*PAAESLEEPDAAAGLSSTK | GP: AK022759_1 |
| DSALAEAPEGLS*PAPPAR | GP: AK022759_1 |
| SEDPPGQEAGS*EEEGSSASGLAK | GP: AK023003_1 |
| LAQTT*PVDSALGSSR | GP: AK023056_1 |
| NLS*GSTLYPVSNIPR | GP: AK023056_1 |
| AAGGAPS*PPPPVR | GP: AK023192_1 |
| FLES*PSR | GP: AK023370_1 |
| TVS*DNSLSNSR | GP: AK023681_1 |
| GS*PEEELPLPAFEK | GP: AK024269_1 |
| TPPT*PPSSIVAK | GP: AK024290_1 |
| EGS*ASTEVLR | GP: AK024391_1 |
| ES*DEDTEDASETDLAK | GP: AK024460_1 |
| STETSDFENIES*PLNER | GP: AK027362_1 |
| GDLS*DVEEEEEEEMDVDEATGAVK | GP: AK027559_1 |
| AAVLS*DSEDEEK | GP: AK027561_1 |
| DSDS*ESEER | GP: AK027561_1 |
| GPASDS*ETEDASR | GP: AK027561_1 |
| KAAVLS*DS*EDEEK | GP: AK027561_1 |
| MSDS*ESEELPKPQVSDSES*EEPPR | GP: AK027561_1 |
| SPAS*DSETEDALKPQIS*DSESEEPPR | GP: AK027561_1 |
| TIAS*DS*EEEAGKELSDK | GP: AK027561_1 |
| TIASDS*EEEAGK | GP: AK027561_1 |
| VVSDADDSDS*DAVSDK | GP: AK027561_1 |
| VVSDADDSDSDAVS*DK | GP: AK027561_1 |
| AAS*PPASASDLIEQQQK | GP: AK027649_1 |
| S*PGHHR | GP: AK027842_1 |
| TVFS*PTLPAAR | GP: AK055851_1 |
| SSPSLDSGDS*DSEELPTFAFLK | GP: AK055926_1 |
| GLFQDEDS*CSDCSYR | GP: AK055931_1 |
| DEASS*VTR | GP: AK056632_1 |
| DPHS*PEDEEQPQGLS*DDDILR | GP: AK056632_1 |
| SQDQDS*EVNELSR | GP: AK056632_1 |
| SQDQDSEVNELS*R | GP: AK056632_1 |
| TQS*PGGCSAEAVLAR | GP: AK056946_1 |
| TSGAPGS*PQTPPER | GP: AK056946_1 |
| GT*PPPVFTPPLPK | GP: AK074638_1 |
| GPEDYPEEGVEES*S*GEASKYTEEDPSGETLSSENK | GP: AK074719_1 |
| WLIS*PVK | GP: AK074809_1 |
| WVEENVPSSVTDVALPALLDS*DEER | GP: AK074870_1 |
| GGS*PDLWK | GP: AK074894_1 |
| GQESSS*DQEQVDVESIDFSK | GP: AK074894_1 |
| LAPVPS*PEPQKPAPVS*PESVK | GP: AK074894_1 |
| SPAGS*PELR | GP: AK074894_1 |
| SSSVSPSSWKS*PPAS*PESWK | GP: AK074894_1 |
| TAPPAS*PEAR | GP: AK074894_1 |
| TTS*PEPR | GP: AK074894_1 |
| HNGVGGS*PPK | GP: AK074903_1 |
| YMNSDTTS*PELR | GP: AK074903_1 |
| FPEFCSSPS*PPVEVK | GP: AK074979_1 |
| GQSS*PPPAPPICLR | GP: AK090617_1 |
| EEAS*DDDMEGDEAVVR | GP: AK090671_1 |
| RS*PPS*PR | GP: AK091273_1 |
| AVT*PVPTK | GP: AK091465_1 |
| GLS*ASLPDLDSENWIEVK | GP: AK091465_1 |
| GLSAS*LPDLDSENWIEVK | GP: AK091465_1 |
| NTFTAWS*DEESDYEIDDR | GP: AK091465_1 |
| SLPTTVPES*PNYR | GP: AK091465_1 |
| STFVQSPADACTPPDTSSAS*EDEGS*LRR | GP: AK091597_1 |
| NTS*PEENLR | GP: AK092570_1 |
| AAALQALQAQAPTS*PPPPPPPLK | GP: AK092772_1 |
| DGDLLS*PSLR | GP: AK092807_1 |
| AFVEDS*EDEDGAGEGGSSLLQK | GP: AK093879_1 |
| RS*TS*PIIGSPPVR | GP: AK094193_1 |
| STS*PIIGSPPVR | GP: AK094193_1 |
| SFNSDSPSIIGVPSETQTS*PVER | GP: AK096613_1 |
| GSGVAQSPQQPPPQQQQQQPPQQPT*PPK | GP: AK096644_1 |
| VNDAEPGS*PEAPQGK | GP: AK097078_1 |
| TLDSDISCPLLESDLAYS*DDDVPSVYENGLSQK | GP: AK097133_1 |
| MGGPRGSGGS*GGGGGR | GP: AK097337_1 |
| SFS*ADNFIGIQR | GP: AK097751_1 |
| GPVSQNS*EVGEEETSAGQGLSSR | GP: AK122582_1 |
| SGIETFS*PPPPPPK | GP: AK122582_1 |
| SSVASGPIS*PTNYR | GP: AL121829_7 |
| EPSPTT*PK | GP: AL133553_2 |
| NSAIS*PQK | GP: AL136109_1 |
| SASSEEASES*PTAR | GP: AL136450_1 |
| TS*PVPK | GP: AL136867_1 |
| AEFTS*PPSLFK | GP: AL136910_1 |
| AES*PESSAIESTQSTPQK | GP: AL137201_1 |
| METVSNASSSSNPSS*PGR | GP: AL137201_1 |
| AQQCVS*PSSSLCR | GP: AL713775_1 |
| GPRT*PS*PPPPIPEDIALGK | GP: AL831833_1 |
| TPS*PPPPIPEDIALGK | GP: AL831833_1 |
| TSAVSS*PLLDQQR | GP: AL831833_1 |
| TFLEGDWTS*PSK | GP: AL831838_1 |
| CS*PTVAFVEFPSSPQLK | GP: AL831962_1 |
| DDSFDSLDS*FGSR | GP: AL831962_1 |
| QQS*LPPPK | GP: AL831962_1 |
| QTPS*PDVVLR | GP: AL831962_1 |
| S*PEPEATLTFPFLDK | GP: AL831962_1 |
| SDSLS*PPR | GP: AL831962_1 |
| DLSTS*PKPSPIPS*PVLGR | GP: AL833968_1 |
| AAEAAPPT*QEAQGETEPTEQAPDALEQAADTSR | GP: AL834162_1 |
| ISDS*ESEDPPR | GP: AL834178_1 |
| NQAS*DS*ENEELPKPR | GP: AL834178_1 |
| VS*DSESEGPQK | GP: AL834178_1 |
| VSDS*ESEGPQK | GP: AL834178_1 |
| TGWDTSESELS*EGELER | GP: AL834216_1 |
| FSTYSQS*PPDTPSLR | GP: AL834312_1 |
| AAEEQGDDQDS*EK | GP: AL834470_1 |
| S*GDETPGSEVPGDK | GP: AL834470_1 |
| SGDET*PGSEVPGDK | GP: AL834470_1 |
| TVS*PSTIR | GP: AL834476_1 |
| SDS*GGSSSEPFDR | GP: AP000505_1 |
| SSVKT*PETVVPAAPELQPPTSTDQPVTPEPTSR | GP: AP000512_4 |
| HSVTAAT*PPPS*PTSGESGDLLSNLLQSPSSAK | GP: AY026388_1 |
| HSVTAAT*PPPSPTSGES*GDLLSNLLQSPSSAK | GP: AY026388_1 |
| ASSQVLSES*PSQDSLDAFMSEMK | GP: AY028435_1 |
| NWEDEDFYDS*DDDTFLDR | GP: AY028435_1 |
| FQSPQIQATIS*PPLQPK | GP: AY036974_1 |
| EAEALLQSMGLTPESPIVPPPMS*PSSK | GP: AY037160_1 |
| DSLGDFIEHYAQLGPSS*PEQLAAGAEEGGGPR | GP: AY039216_1 |
| RGGGSGGGEES*EGEEVDED | GP: AY039216_1 |
| ALS*PVTSR | GP: AY044869_1 |
| LPASPSGSEDLSSVSSS*PTSSP | GP: AY050169_1 |
| FLTDT*SHLLSAVR | GP: AY061759_1 |
| MEISAELPQT*PQR | GP: AY061886_1 |
| AFAAVPTSHPPEDAPAQPPTPGPAAS*PEQLSFR | GP: AY062238_1 |
| MAESPCSPSGQQPPSPPS*PDELPANVK | GP: AY062238_1 |
| NS*LESISSIDR | GP: AY062238_1 |
| QSPAS*PPPLGGGAPVR | GP: AY062238_1 |
| VQS*PEPPAPER | GP: AY062238_1 |
| VS*PTGAAGR | GP: AY062238_1 |
| AAVFIQS*K | GP: AY101367_1 |
| QGGSQPSSFS*PGQSQVTPQDQEK | GP: AY130299_1 |
| ATNES*EDEIPQLVPIGK | GP: AY154473_1 |
| LSSPAAFLPACNS*PSK | GP: AY166851_1 |
| ASS*LNVLNVGGK | GP: AY180166_1 |
| RPPS*PDVIVLS*DNEQPSSPR | GP: AY186731_1 |
| RPPS*PDVIVLSDNEQPSS*PR | GP: AY186731_1 |
| TLS*SSAQEDIIR | GP: AY190323_1 |
| VTETEDDS*DS*DS*DDDEDDVHVTIGDIK | GP: AY229892_1 |
| GDSDIS*DEEAAQQSK | GP: AY283618_1 |
| GNIETTSEDGQVFS*PK | GP: AY283618_1 |
| S*KGDSDIS*DEEAAQQSK | GP: AY283618_1 |
| S*LS*PSHLTEDR | GP: AY283618_1 |
| SAS*PYPSHSLSS*PQR | GP: AY283618_1 |
| TPS*PSYQR | GP: AY283618_1 |
| GPQPPTVS*PIR | GP: BC000656_1 |
| NNS*GEEFDCAFR | GP: BC001041_1 |
| TPAPPEPGS*PAPGEGPSGR | GP: BC001728_1 |
| GAFMLEPEGMSPMEPAGVS*PMPGTQK | GP: BC001937_1 |
| SSS*ESYTQSFQSR | GP: BC003167_1 |
| DLFSLDSEDPSPAS*PPLR | GP: BC003640_1 |
| GFSQYGVSGS*PTK | GP: BC005883_1 |
| WTVHTGEKS*FGCNEYGK | GP: BC006258_1 |
| ATDSDLSS*PR | GP: BC006350_1 |
| NSKYEYDPDIS*PPR | GP: BC006350_1 |
| SSDSDLS*PPR | GP: BC006350_1 |
| YEYDPDIS*PPR | GP: BC006350_1 |
| LYSILQGDS*PTK | GP: BC006474_1 |
| SAS*PDDDLGSSNWEAADLGNEER | GP: BC007103_1 |
| AAS*PESASSTPESLQAR | GP: BC007642_1 |
| NDQEPPPEALDFS*DDEKEK | GP: BC008207_1 |
| SRIPS*PLQPEMQGTPDDEPSEPEPS*PSTLIYR | GP: BC009071_1 |
| SPITSS*PPK | GP: BC009539_1 |
| EEVGAGYNS*EDEYEAAAAR | GP: BC009917_1 |
| SSYANVFGDGPYSTFLTSS*PIR | GP: BC010629_1 |
| STLS*PPEASPGPPAAPR | GP: BC011630_1 |
| ALS*IFVGLFNIEETNDNIQIVIK | GP: BC013576_1 |
| S*PPYEGK | GP: BC014394_1 |
| SVNEILGLAESS*PNEPK | GP: BC014658_1 |
| IGELGAPEVWGLS*PK | GP: BC015354_1 |
| FQSQADQDQQASGLQS*PPSR | GP: BC016029_1 |
| VSSPLSPLS*PGIKS*PTIPR | GP: BC016029_1 |
| SS*PQLDPLR | GP: BC016842_1 |
| S*VSPSPVPLSSNYIAQISNGQQLMSQPQLHR | GP: BC017705_1 |
| SNS*CSSISVASCISEWEQK | GP: BC017705_1 |
| VENSPQVDGS*PPGLEGLLGGIGEK | GP: BC018184_1 |
| FELEASLATLLLGLSNVTVIS*LAET*KDIPAAILHAFLR | GP: BC018426_1 |
| SGISTNHADYSSS*PAGS*PGAQVSLYNSPSVASPAR | GP: BC018775_1 |
| LVGLNLS*PPMSPVQLPLR | GP: BC019232_1 |
| NSNSPPS*PSSMNQR | GP: BC020516_1 |
| QELGS*PEER | GP: BC020567_1 |
| EPAFEDITLES*ER | GP: BC027178_1 |
| ELSDQATAS*PIVAR | GP: BC028697_1 |
| LTQTSST*EQLNVLETETEVLNK | GP: BC028697_1 |
| SSS*PVQVEEEPVR | GP: BC029266_1 |
| NDS*GEENVPLDLTR | GP: BC029608_1 |
| ACAS*PSAQVEGSPVAGSDGSQPAVK | GP: BC030547_1 |
| ACASPSAQVEGS*PVAGSDGSQPAVK | GP: BC030547_1 |
| S*PGLCSDSLEK | GP: BC030687_1 |
| LSS*EDEEEDEAEDDQSEASGKK | GP: BC030817_1 |
| ETAVQCDVGDLQPPPAKPAS*PAQVQSSQDGGCPK | GP: BC032244_1 |
| EVDFDS*DPMEECLR | GP: BC032244_1 |
| ASALGLGDGEEEAPPSRS*DPDGGDS*PLPASGGPLTCK | GP: BC032463_1 |
| ATDIPASAS*PPPVAGVPFFKQS*PGHQS*PLASPK | GP: BC032463_1 |
| AVVLPGGTATS*PK | GP: BC032463_1 |
| SDPDGGDS*PLPASGGPLTCK | GP: BC032463_1 |
| TASISSS*PSEGTPTVGSYGCTPQSLPK | GP: BC033856_1 |
| S*PEAVGPELEAEEK | GP: BC035076_1 |
| VTPLQSPIDKPSDSLSIGNGDNSQQISNSDTPS*PPPGLSK | GP: BC035590_1 |
| AKS*PTPS*PSPPRNS*DQEGGGK | GP: BC036187_1 |
| AKS*PTPSPS*PPR | GP: BC036187_1 |
| AKSPTPS*PS*PPR | GP: BC036187_1 |
| EPSVQEAT*STSDILK | GP: BC036187_1 |
| GASSS*PQR | GP: BC036187_1 |
| GSS*PSRS*TR | GP: BC036187_1 |
| SPTPSPS*PPRNS*DQEGGGK | GP: BC036187_1 |
| SATDGNTSTT*PPTSAK | GP: BC036216_1 |
| AVS*PLDPSK | GP: BC036831_1 |
| ALEEGDGSVSGSS*PR | GP: BC037404_1 |
| ATS*PESTSR | GP: BC037404_1 |
| IDENS*DKEMEVEES*PEK | GP: BC037404_1 |
| TGTDSNSTESSETST*GSLCK | GP: BC037404_I |
| ALSAAVADSLTNS*PR | GP: BC037556_1 |
| YSPDEMNNS*PNFEEK | GP: BC037556_1 |
| LLS*PLSSAR | GP: BC037565_1 |
| TVLPTVPES*PEEEVK | GP: BC038513_1 |
| VESSENVPSPTHPPVVINAADDDEDDDDQFS*EEGDETK | GP: BC038513_1 |
| TNLTSQSSTTNLPGSPGSPGSPGS*PGSPGSVPK | GP: BC038932_1 |
| VEVTPT*VPR | GP: BC039295_1 |
| AAS*DDGSLK | GP: BC039612_1 |
| GWAFGSNS*LPIAGSVGMGVAR | GP: BC039652_1 |
| SRS*PES*QVIGENTK | GP: BC039814_1 |
| SYSSSSSS*PER | GP: BC039814_1 |
| DS*ENTPVK | GP: BC039843_1 |
| EMDESLANLS*EDEYYSEEER | GP: BC040194_1 |
| EMDESLANLSEDEYYS*EEER | GP: BC040194_1 |
| ARPQPSGPAPSS* | GP: BC041166_1 |
| AEAPSS*PDVAPAGK | GP: BC041631_1 |
| TAVQYIESS*DSEEIETSELPQK | GP: BC044659_1 |
| ASIGQS*PGLPSTTFK | GP: BC045623_1 |
| DVEDMELS*DVEDDGSK | GP: BC045623_1 |
| IIS*PGSSTPSSTR | GP: BC045623_1 |
| LESESTS*PSLEMK | GP: BC045623_1 |
| SAT*PEPVTDNR | GP: BC045623_1 |
| SFNYS*PNSSTSEVSSTSASK | GP: BC045623_1 |
| SDS*APPTPVNR | GP: BC047482_1 |
| TSDDEVGS*PK | GP: BC047529_1 |
| LPPPPPQAPPEEENES*EPEEPSGVEGAAFQSR | GP: BC048134_1 |
| AS*DLEDEESAAR | GP: BC050463_1 |
| DSGS*DQDLDGAGVR | GP: BC050463_1 |
| DSGS*DQDLDGAGVRAS*DLEDEESAAR | GP: BC050463_1 |
| GPTSS*PCEEEGDEGEEDRT*SDLR | GP: BC050463_1 |
| KLGVS*VS*PSR | GP: BC050463_1 |
| KLGVS*VSPS*R | GP: BC050463_1 |
| LGVSVS*PSR | GP: BC050463_1 |
| S*PAPAQTR | GP: BC050463_1 |
| S*PQPPSR | GP: BC050463_1 |
| TLSGSGSGSGSSYSGSSS*R | GP: BC050463_1 |
| TSAS*SASASNSSR | GP: BC050463_1 |
| TSASSASAS*NSSR | GP: BC050463_1 |
| LFPS*PGLPTR | GP: BC050553_1 |
| SDS*DSSTLAK | GP: BC053873_1 |
| TLSLTSLGLS*MPADPCEGGAR | GP: BX248266_1 |
| SFLVASVLPGPDGNINS*PTR | GP: BX537838_1 |
| VTENGGS*PQGIK | GP: D49835_1 |
| CASSESDS*DENQNK | GP: D63875_1 |
| GGEFDEFVNDDT*DDDLPISK | GP: D63875_1 |
| GS*DNEGSGQGSGNESEPEGSNNEASDR | GP: D63875_1 |
| GS*GSEQEGEDEEGGER | GP: D63875_1 |
| GSDNEGSGQGS*GNESEPEGSNNEASDR | GP: D63875_1 |
| GSDNEGSGQGSGNESEPEGS*NNEASDR | GP: D63875_1 |
| KGS*GS*EQEGEDEEGGER | GP: D63875_1 |
| NS*NSNSDSDEDEQR | GP: D63875_1 |
| NSNS*NSDSDEDEQR | GP: D63875_1 |
| NSNSNSDS*DEDEQR | GP: D63875_1 |
| SGSEAGS*PR | GP: D63875_1 |
| GAPSS*PATGVLPSPQGK | GP: D79991_1 |
| AVIVSS*PK | GP: D83032_1 |
| SES*LSNCSIGK | GP: D86982_1 |
| VVIDSDTEDSGS*DENLDQELLSLAK | GP: D87440_1 |
| T*GGGGSGGGGSGGGGSDVK | GP: L43067_1 |
| GEGGILLSS*PGGPTTDK | GP: S74786_1 |
| S*AEDELAMR | GP: U07561_1 |
| CETS*PPSSPR | GP: U22815_1 |
| GVELCFPENET*PPEGK | GP: U49844_1 |
| IGGDAATT*GNNSTPDFGFGGQK | GP: U69126_1 |
| S*APTTPK | GP: U70136_1 |
| ADS*LLAVVK | GP: U72355_1 |
| NFWVSGLSST*TR | GP: U72355_1 |
| S*VVSFDK | GP: U72355_1 |
| SVVS*FDK | GP: U72355_1 |
| DLDEEGS*EK | GP: U76992_1 |
| LFDDS*DER | GP: U76992_1 |
| LFDEEEDS*S*EKLFDDSDER | GP: U76992_1 |
| LFEDDDS*NEK | GP: U76992_1 |
| LFEES*DDKEDEDADGK | GP: U76992_1 |
| VFDDES*DEKEDEEYADEK | GP: U76992_1 |
| VLDEEGS*ER | GP: U76992_1 |
| VLDEEGS*EREFDEDS*DEKEEEEDTYEK | GP: U76992_1 |
| S*ISESSR | GP: U77718_1 |
| VQIS*PDSGGLPER | GP: U94832_1 |
| TPS*PSQPK | GP: U95825_1 |
| RS*PQQTVPYVVPLS*PK | GP: Y18004_1 |
| SPQQTVPYVVPLS*PK | GP: Y18004_1 |
| QLEDIINTYGSAAS*TAGKEGS*AR | GPN: AB085905_1 |
| IES*DEEEDFENVGK | GPN: AF227948_1 |
| CSSSSGGGSS*GDEDGLELDGAPGGGK | GPN: AJ421269_1 |
| LEDLDTCMMT*PK | GPN: AK000055_1 |
| AVET*PPLSSVNLLEGLSR | GPN: AK000126_1 |
| LPSS*EPDAPRLLRS*PVTCTPK | GPN: AK000538_1 |
| TPSSS*PPITPPASETK | GPN: AK000742_1 |
| ISSSFFFFLRQS*LTLSPR | GPN: AK025116_1 |
| STDSSSYPSPCASPS*PPSSGK | GPN: AK025593_1 |
| VDGIPNDSSDS*EMEDK | GPN: AK025593_1 |
| LQQGAGLESPQGQPEPGAAS*PQR | GPN: AK025974_1 |
| QEVVST*AGPR | GPN: AK026010_1 |
| S*PGYESESSR | GPN: AK027089_1 |
| SPGLVPPS*PEFAPR | GPN: AK027089_1 |
| SPVQEASSATDTDTNS*QEDPADTASVSSLSLS*TGHTK | GPN: AK074370_1 |
| AIS*PSIK | GPN: AK093809_1 |
| LSST*PPLSALGR | GPN: AK093809_1 |
| S*LSSPTVTLSAPLEGAK | GPN: AY312514_1 |
| SS*PEQPIGQGR | GPN: AY358482_1 |
| GS*GGS*SGDELREDDEPVK | GPN: AY358600_1 |
| VEEEQEADEEDVS*EEEAESK | GPN: AY358640_1 |
| VPVLMES*R | GPN: AY358941_1 |
| GQPGNAYDGAGQPSAAYLSMSQGAVANANST*PPPYER | GPN: BC000488_1 |
| QPT*PPFFGR | GPN: BC000488_1 |
| AGEPNS*PDAEEANS*PDVTAGCDPAGVHPPR | GPN: BC001041_1 |
| ESTQLS*PADLTEGKPTDPSK | GPN: BC001041_1 |
| VDIPS*PPPR | GPN: BC001044_1 |
| SAS*SDTSEELNSQDSPPK | GPN: BC001443_1 |
| YLFNQLFGEEDADQEVS*PDR | GPN: BC003153_1 |
| ALPSLNTGSSS*PR | GPN: BC003553_1 |
| LDSQPQETS*PELPR | GPN: BC003553_1 |
| TLEEVVMAEEEDEGTDRPGS*PA | GPN: BC007448_1 |
| GDSES*EEDEQDSEEVR | GPN: BC007664_1 |
| QLEEPGAGTPS*PVR | GPN: BC008084_1 |
| TEDGGWEWS*DDEFDEESEEGK | GPN: BC008726_1 |
| AQPGAAPGIYQQSAEASSS*QGTAANSQSYTIMSPAVLK | GPN: BC008733_1 |
| AQVPGPLT*PEMEAR | GPN: BC008948_1 |
| LAAQLGAPTS*PIPDSAIVNTR | GPN: BC008948_1 |
| QS*PPIVK | GPN: BC009039_1 |
| ILDEDSWS*DGEQEPITVDQTWR | GPN: BC009746_1 |
| ESLPPAAAAEPS*PVSK | GPN: BC010907_1 |
| DTSATSQSVNGS*PQAEQPSLESTSK | GPN: BC011551_1 |
| VFVGGLS*PDTSEEQIK | GPN: BC011714_1 |
| S*GSLGSAR | PIR2: T00257 |
| SAPSS*PAPR | PIR2: T00257 |
| EPPS*PADVPEK | PIR2: T00262 |
| AGNS*DSEEDDANGR | PIR2: T00347 |
| AGNSDS*EEDDANGR | PIR2: T00347 |
| QLVLETLYALTSS*TKIIK | PIR2: T00361 |
| LSLTSDPEEGDPLALGPES*PGEPQPPQLK | PIR2: T00363 |
| SS*LSGDEEDELFK | PIR2: T00363 |
| SSLS*GDEEDELFK | PIR2: T00363 |
| LSVQSNPS*PQLR | PIR2: T00368 |
| DGGAAS*PATEGR | PIR2: T00387 |
| S*PTGSTTSR | PIR2: T00387 |
| SDIDVNAAAS*AK | PIR2: T00387 |
| SIS*LGDSEGPIVATLAQPLR | PIR2: T01437 |
| QEPQS*PSR | PIR2: T02672 |
| ALS*PVIPLIPR | PIR2: T03454 |
| EGAASPAPETPQPTS*PETSPK | PIR2: T08760 |
| TTHLAGALS*PGEAWPFESV | PIR2: T08760 |
| AETASQSQRS*PISDNSGCDAPGNSNPSLSVPSSAESEK | PIR2: T09073 |
| LESS*EGEIIQTVDR | PIR2: T09073 |
| QDQISGLS*QSEVK | PIR2: T09073 |
| S*PISDNSGCDAPGNSNPSLSVPSSAESEK | PIR2: T09073 |
| SSS*NDSVDEETAESDTSPVLEK | PIR2: T09073 |
| SSSNDS*VDEETAESDTSPVLEK | PIR2: T09073 |
| SSSNDSVDEETAES*DTSPVLEK | PIR2: T09073 |
| SSSNDSVDEETAESDTS*PVLEK | PIR2: T09073 |
| SSVAAPEKSS*S*NDSVDEETAESDTSPVLEK | PIR2: T09073 |
| VGSSSS*ESCAQDLPVLVGEEGEVK | PIR2: T09073 |
| GGAGAWLGGPAASLS*PPK | PIR2: T09219 |
| GTPGS*PSGTQEPR | PIR2: T09219 |
| SLS*PDEER | PIR2: T12518 |
| LFQGYS*FVAPSILFK | PIR2: T13149 |
| APQQQPPPQQPPPPQPPPQQPPPPPSYS*PAR | PIR2: T13159 |
| NYILDQTNVYGS*AQR | PIR2: T13159 |
| SFLSEPSS*PGR | PIR2: T17232 |
| RAAAS*PPS*R | PIR2: T41998 |
| CS*ATPSAQVKPIVSAS*PPSR | PIR2: T46375 |
| ETEAAPTS*PPIVPLK | PIR2: T46385 |
| TGDLGIPPNPEDRS*PS*PEPIYNSEGK | PIR2: G02919 |
| ASWAS*ENGETDAEGTQMTPAK | PIR2: I38414 |
| GYYS*PGIVSTR | PIR2: I38414 |
| KNS*STDQGS*DEEGSLQK | PIR2: I38414 |
| NSSTDQGS*DEEGSLQK | PIR2: I38414 |
| TSQPPVPQGEAEEDS*QGK | PIR2: I38414 |
| GPGQVPTATSALSLELQEVEPLGLPQAS*PSR | PIR2: I52882 |
| TRS*PDVISSASTALSQDIPEIASEALSR | PIR2: I52882 |
| S*PS*PKPTK | PIR2: JC4525 |
| SSSSSSSSGSPS*PSR | PIR2: JC4525 |
| EEAGETS*PADESGAPK | PIR2: J07079 |
| STTPCMVLASEQDPDLELISDLDEGPPVLT*PVENTR | PIR2: JC7079 |
| QSNASS*DVEVEEK | PIR2: JC7168 |
| SLS*PQEDALTGSR | PIR2: JC7680 |
| QPPGVPNGPSS*PTNESAPELPQR | PIR2: JC7807 |
| RGSS*S*DEEGGPK | PIR2: JW0057 |
| AVSTVVVTTAPS*PK | PIR2: S52863 |
| S*PSPAVPLR | PIR2: S52863 |
| SEAEDLAEPLSSTEGVAPLSQAPS*PLAIPAIK | PIR2: S52863 |
| SPS*PAVPLR | PIR2: S52863 |
| SMSSIPPYPASSLASSS*PPGSGR | PIR2: S55553 |
| AT*PPPSPLLSELLK | PIR2: S68142 |
| GSLLPTS*PR | PIR2: S68142 |
| S*PVGSGAPQAAAPAPAAHVAGNPGGDAAPAATGTAAAASLATAAGS | PIR2: S69501 |
| EDAEK | |
| LASEYLT*PEEMVTFK | PIR2: T00034 |
| SANGGS*ESDGEENIGWSTVNLDEEK | PIR2: T00034 |
| CGGVEQASSS*PR | PIR2: T00059 |
| GPLEPS*EPAVVAAAR | DNA-3-methyladenine glycosylase |
| SLS*PGK | ATP-binding cassette, sub-family B, member 9 precursor |
| TDEVPAGGS*RS*EAEDEDDEDYVPYVPLR | DEAD-box protein abstrakt homolog |
| ELS*QNTDESGLNDEAIAK | Activator 1 140 kDa subunit |
| IIYDS*DS*ESEETLQVK | Activator 1 140 kDa subunit |
| QDPVTYIS*ETDEEDDFMCK | Activator 1 140 kDa subunit |
| ASLVALPEQTASEEET*PPPLLTK | Apoptotic chromatin condensation inducer in the nucleus |
| DPSSGQEVAT*PPVPQLQVCEPK | Apoptotic chromatin condensation inducer in the nucleus |
| DS*STSYTETKDPSSGQEVATPPVPQLQVCEPK | Apoptotic chromatin condensation inducer in the nucleus |
| DSSTSYTETKDPSS*GQEVATPPVPQLQVCEPK | Apoptotic chromatin condensation inducer in the nucleus |
| DSSTSYTETKDPSSGQEVAT*PPVPQLQVCEPK | Apoptotic chromatin condensation inducer in the nucleus |
| LS*EGSQPAEEEEDQETPSR | Apoptotic chromatin condensation inducer in the nucleus |
| LSEGS*QPAEEEEDQETPSR | Apoptotic chromatin condensation inducer in the nucleus |
| SKS*PS*PPR | Apoptotic chromatin condensation inducer in the nucleus |
| SLS*PGVSR | Apoptotic chromatin condensation inducer in the nucleus |
| SLSPGVS*R | Apoptotic chromatin condensation inducer in the nucleus |
| SPS*PPR | Apoptotic chromatin condensation inducer in the nucleus |
| TAQVPS*PPR | Apoptotic chromatin condensation inducer in the nucleus |
| TTS*PLEEEER | Apoptotic chromatin condensation inducer in the nucleus |
| TAS*FSESR | ATP-citrate synthase |
| GDEASEEGQNGSS*PK | Alpha adducin |
| SPGS*PVGEGTGSPPK | Alpha adducin |
| IEEVLSPEGSPS*KS*PSK | Gamma adducin |
| ELSPLISLPS*PVPPLSPIHS*NQQTLPR | AF-4 protein |
| IT*LDLLSR | AF-4 protein |
| RPGS*VSST*DQER | AF-4 protein |
| S*PAQQEPPQR | AF-4 protein |
| ITSVS*TGNLCTEEQTPPPRPEAYPIPTQTYTR | AF-6 protein |
| SSPNVANQPPS*PGGK | AF-6 protein |
| AS*LGSLEGEAEAEASSPK | Neuroblast differentiation associated protein AHNAK |
| ASLGS*LEGEAEAEASSPK | Neuroblast differentiation associated protein AHNAK |
| GGVTGS*PEASISGSK | Neuroblast differentiation associated protein AHNAK |
| IS*APNVDFNLEGPK | Neuroblast differentiation associated protein AHNAK |
| ISMQDVDLSLGS*PK | Neuroblast differentiation associated protein AHNAK |
| LGS*PSGK | Neuroblast differentiation associated protein AHNAK |
| SNS*FSDER | Neuroblast differentiation associated protein AHNAK |
| VKGS*LGATGEIKGPTVGGGLPGIGVQGLEGNLQMPGIK | Neuroblast differentiation associated protein AHNAK |
| VDSEGDFS*ENDDAAGDFR | A-kinase anchor protein 8 |
| AIT*PPLPESTVPFSNGVLK | A kinase anchor protein 1, mitochondrial precursor |
| SNILSDNPDFS*DEADIIK | Acidic nucleoplasmic DNA-binding protein 1 |
| LAS*PELER | Transcription factor AP-1 |
| EWSLESSPAQNWT*PPQPR | ADP-ribosylation factor GTPase activating protein 1 |
| MS*GFIYQGK | Rho guanine nucleotide exchange factor 6 |
| TQLWASEPGT*PPLPTSLPSQNPILK | Arsenite-resistance protein 2 |
| SSGNSSSSGSGSGSTSAGSSS*PGAR | Aspartyl/asparaginyl beta-hydroxylase |
| EFDELNPS*AQR | Sarcoplasmic/endoplasmic reticulum calcium ATPase 2 |
| MPLDLS*PLATPIIR | Cyclic-AMP-dependent transcription factor ATF-2 |
| SLAFEEGS*QSTTISSLSEK | Serine-protein kinase ATM |
| TSS*PPR | Transcriptional regulator ATRX |
| CS*PSSSSINNS*SSKPT*K | Ataxin-7 |
| LAEDEGDS*EPEAVGQSR | Bromodomain adjacent to zinc finger domain protein 1B |
| SDVQEES*EGS*DTDDNKDSAAFEDNEVQDEFLEK | Bromodomain adjacent to zinc finger domain protein 1B |
| AS*PVTSPAAAFPTASPANK | Bromodomain adjacent to zinc finger domain 2A |
| AS*PPLQDSASQTYESMCLEK | Transcription regulator protein BACH1 |
| ISES*PEPGQR | Transcription regulator protein BACH1 |
| SQS*PAASDCSSSSSSASLPSSGR | BAG-family molecular chaperone regulator-3 |
| SSVQGASS*REGS*PAR | BAG-family molecular chaperone regulator-3 |
| VPPAPVPCPPPS*PGPSAVPSSPK | BAG-family molecular chaperone regulator-3 |
| VPPAPVPCPPPSPGPSAVPSS*PK | BAG-family molecular chaperone regulator-3 |
| EGPEPPEEVPPPTT*PPVPK | Large proline-rich protein BAT2 |
| GNS*PNSEPPTPK | Large proline-rich protein BAT2 |
| LIPGPLS*PVAR | Large proline-rich protein BAT2 |
| AS*PEPQRENAS*PAPGTTAEEAMSR | Large proline-rich protein BAT3 |
| ENAS*PAPGTTAEEAMSR | Large proline-rich protein BAT3 |
| LQEDPNYS*PQR | Large proline-rich protein BAT3 |
| T*PTAVQVK | BCE-1 protein |
| AVT*PVSQGSNSSSADPK | B-cell lymphoma 9 protein |
| IPVEGPLS*PSR | B-cell lymphoma 9 protein |
| LSVSSNDT*QESGNSSGPSPGAK | Brefeldin A-inhibited guanine nucleotide-exchange |
| | protein 1 |
| LDS*T*QVGDFLGDSAR | Brefeldin A-inhibited guanine nucleotide-exchange |
| | protein 2 |
| GNKS*PS*PPDGSPAATPEIR | Myc box dependent interacting protein 1 |
| GNKS*PSPPDGS*PAATPEIR | Myc box dependent interacting protein 1 |
| SPS*PPDGSPAATPEIR | Myc box dependent interacting protein 1 |
| YSEWTSPAEDSS*PGISLSSSR | Bloom's syndrome protein |
| ADTTTPTPTAILAPGS*PASPPGSLEPK | Bromodomain-containing protein 2 |
| KADTTTPTPTAILAPGS*PAS*PPGSLEPK | Bromodomain-containing protein 2 |
| QASASYDS*EEEEEGLPMSYDEK | Bromodomain-containing protein 3 |
| SES*PPPLSDPK | Bromodomain-containing protein 3 |
| MPDEPEEPVVAVSS*PAVPPPTK | Bromodomain-containing protein 4 |
| TEGVS*PIPQEIFEYLMDR | Peregrin |
| VAVEYLDPS*PEVQK | Mitotic checkpoint protein BUB3 |
| YNAS*SFAK | Cadherin-17 precursor |
| LNSEAS*PSR | Chromatin assembly factor 1 subunit A |
| S*CPELTSGPR | Chromatin assembly factor 1 subunit A |
| TDTPPSSVPTSVISTPSTEEIQSETPGDAQGS*PPELK | Chromatin assembly factor 1 subunit B |
| TQDPSS*PGTTPPQAR | Chromatin assembly factor 1 subunit B |
| S*PPSLR | Signal transduction protein CBL-C |
| SIS*PSALQDLLR | CREB-binding protein |
| QGQSQAASSSSVTS*PIK | Cyclin T2 |
| ES*EHDSDESS*DDDS*DSEEPSK | Leukocyte common antigen precursor |
| IGEGT*YGVVYK | Cell division protein kinase 2 |
| VSNGS*PSLER | Cyclin-dependent kinase inhibitor 1B |
| KSS*PSTGS*LDSGNESK | Centaurin beta 2 |
| ATPATAPGTS*PR | Centaurin gamma 3 |
| VQEHEDS*GDS*EVENEAK | WD-repeat protein CGI-48 |
| EVQAEQPSSSS*PR | Hypothetical protein CGI-79 |
| ELQGDGPPSS*PTNDPTVK | Chromodomain helicase-DNA-binding protein 3 |
| METEADAPS*PAPSLGER | Chromodomain helicase-DNA-binding protein 3 |
| MSQPGS*PSPK | Chromodomain helicase-DNA-binding protein 4 |
| MSQPGSPS*PK | Chromodomain helicase-DNA-binding protein 4 |
| S*DSEGSDYTPGK | Chromodomain helicase-DNA-binding protein 4 |
| STAPETAIECTQAPAPAS*EDEKVVVEPPEGEEK | Chromodomain helicase-DNA-binding protein 4 |
| NIPS*PGQLDPDTR | Probable chromodomain-helicase-DNA-binding protein |
| | KIAA1416 |
| T*PDTIR | Clathrin heavy chain 1 |
| TSIDAYDNFDNIS*LAQR | Clathrin heavy chain 1 |
| RFS*DS*EGEETVPEPR | CLN3 protein |
| SPSDLT*NPER | cAMP-specific 3′,5′-cyclic phosphodiesterase 4C |
| FIIGSVSEDNS*EDEISNLVK | Acetyl-CoA carboxylase 1 |
| DADS*QNPDAPEGK | Coatomer alpha subunit |
| NLS*PGAVESDVR | Coatomer alpha subunit |
| GS*FPVAEKVNK | Cytochrome P450 2C18 |
| SGPEAEGLGSETSPT*VDDEEEMLYGDSGSLFSPSK | Cleavage and polyadenylation specificity factor, 160 kDa |
| | subunit |
| VDTGVILEEGELKDDGEDS*EMQVEAPSDSSVIAQQK | Cleavage and polyadenylation specificity factor, 100 kDa |
| | subunit |
| AIT*PPQQPYK | Cell division cycle 2-related protein kinase 7 |
| DGSGGASGTLQPSSGGGSSNS*R | Cell division cycle 2-related protein kinase 7 |
| GS*PVFLPR | Cell division cycle 2-related protein kinase 7 |
| NSS*PAPPQPAPGK | Cell division cycle 2-related protein kinase 7 |
| QDDSPSGASYGQDYDLS*PSR | Cell division cycle 2-related protein kinase 7 |
| S*PGSTSR | Cell division cycle 2-related protein kinase 7 |
| SPS*PYSR | Cell division cycle 2-related protein kinase 7 |
| SVS*PYSR | Cell division cycle 2-related protein kinase 7 |
| TVDS*PK | Cell division cycle 2-related protein kinase 7 |
| SVNEDDNPPS*PIGGDMMDSLISQLQPPPQQQPFPK | Cofactor required for Sp1 transcriptional activation |
| | subunit 2 |
| FYDLS*DSDSNLSGEDSK | Hypothetical protein C20orf6 |
| IEIPVTPTGQSVPSS*PSIPGTPTLK | Protein C20orf67 |
| TFQQIQEEEDDDYPGSYS*PQDPSAGPLLTEELIK | Protein C20orf77 |
| TTPES*PPYSSGSYDSIK | Hypothetical protein C20orf112 |
| TPEELDDS*DFETEDFDVR | Alpha-1 catenin |
| MQGQS*PPAPTR | CH-TOG protein |
| MLQALS*PK | Cholinephosphate cytidylyltransferase B |
| FLPS*PVVIK | Cullin homolog 3 |
| TPQS*PTLPPAK | Coxsackievirus and adenovirus receptor precursor |
| DAEPPS*PTPAGPPR | Adenylate cyclase, type VI |
| KPS*PQPSS*PR | Cyclin K |
| YT*RNLVDQGNGK | Cysteine dioxygenase type I |
| IS*ATSAEER | Cytohesin 4 |
| ILQEKLDQPVS*APPS*PR | H4 protein |
| LDQPVSAPPS*PR | H4 protein |
| SGVDQMDLFGDMST*PPDLNSPTESK | Disabled homolog 2 |
| SGVDQMDLFGDMSTPPDLNS*PTESK | Disabled homolog 2 |
| SSPNPFVGS*PPK | Disabled homolog 2 |
| ICTLPSPPS*PLASLAPVADSSTR | Death domain-associated protein 6 |
| LLEDS*EESSEETVSR | Putative pre-mRNA splicing factor RNA helicase |
| ISLEQPPNGSDT*PNPEK | Probable ATP-dependent RNA helicase DDX20 |
| YQES*PGIQMK | Probable ATP-dependent RNA helicase DDX20 |
| NGFPHPEPDCNPSEAASEES*NSEIEQEIPVEQK | Nucleolar RNA helicase II |
| AQAVS*EEEEEEEGK | ATP-dependent RNA helicase DDX24 |
| SPGKAEAESDALPDDT*VIESEALPSDIAAEAR | ATP-dependent RNA helicase DDX24 |
| SEEVPAFGVAS*PPPLTDTPDTTANAEGDLPTTMGGPLPPHLALK | ATP-dependent RNA helicase A |
| GPAAPLTPGPQS*PPTPLAPGQEK | Deformed epidermal autoregulatory factor 1 homolog |
| GAGSIAGASAS*PK | Desmoplakin |
| GGGGYTCQS*GSGWDEFTK | Desmoplakin |
| GLPS*PYNMSSAPGSR | Desmoplakin |
| GLPSPYNMSSAPGS*R | Desmoplakin |
| SMS*FQGIR | Desmoplakin |
| SSSFS*DTLEESSPIAAIFDTENLEK | Desmoplakin |
| SSDQPLTVPVS*PK | Restricted expression proliferation associated protein 100 |
| AGLESGAEPGDGDS*DTTK | Dyskerin |
| AKEVELVS*E | Dyskerin |
| HVTS*NAS*DSESSYR | Presynaptic protein SAP97 |
| YHS*LGNISR | Dystrophia myotonica-containing WD repeat motif protein |
| AET*PTESVSEPEVATK | DNA ligase I |
| KQSQIQNQQGEDS*GSDPEDTY | DNA ligase I |
| TIQEVLEEQS*EDEDR | DNA ligase I |
| VLGS*EGEEEDEALSPAK | DNA ligase I |
| VLGSEGEEEDEALS*PAK | DNA ligase I |
| EADDDEEVDDNIPEMPS*PK | DNA (cytosine-5)-methyltransferase 1 |
| LSS*PVK | DNA (cytosine-5)-methyltransferase 1 |
| AIST*PETPLTK | DNA polymerase alpha 70 kDa subunit |
| S*PHQLLSPSSFS*PSATPSQK | DNA polymerase alpha 70 kDa subunit |
| IAS*PVSR | DNA polymerase alpha catalytic subunit |
| LS*S*PVLHR | Drebrin |
| AAAAGLGHPASPGGS*EDGPPGS*EEEDAAR | Dead ringer like-1 protein |
| APS*PGAYK | Atrophin-1 |
| AS*PGGVSTSSSDGK | Atrophin-1 |
| QEPAEEYETPESPVPPARS*PS*PPPK | Atrophin-1 |
| S*LNDDGSSDPR | Atrophin-1 |
| SEEIS*ESESEETNAPK | Atrophin-1 |
| SLNDDGSS*DPR | Atrophin-1 |
| TAS*PPGPPPYGK | Atrophin-1 |
| TAT*PPGYKPGS*PPSFR | Atrophin-1 |
| TEQELPRPQS*PSDLDS*LDGR | Atrophin-1 |
| TGT*PPGYR | Atrophin-1 |
| DFQDYMEPEEGCQGS*PQR | Dynein light intermediate chain 2, cytosolic |
| RS*PTSSPT*PQR | Dynamin-1 |
| EALNIIGDISTSTVSTPVPPPVDDTWLQSASSHSPT*PQR | Dynamin-2 |
| GGS*PQMDDIK | Translation initiation factor elF-2B epsilon subunit |
| EVAENQQNQSS*DPEEEK | Band 4.1-like protein 2 |
| LVS*PEQPPK | Band 4.1-like protein 2 |
| S*LDGAPIGVMDQSLMK | Band 4.1-like protein 2 |
| AAEDDSAS*PPGAASDAEPGDEERPGLQVDCVVCGDK | Orphan nuclear receptor EAR-2 |
| S*STPVPS*K | ECT2 protein |
| YGPADVEDTTGSGATDSKDDDDIDLFGS*DDEEESEEAK | Elongation factor 1-beta |
| FSVS*PVVR | Elongation factor 2 |
| ELVEPLT*PSGEAPNQALLR | Epidermal growth factor receptor precursor |
| GPDEAMEDGEEGS*DDEAEWVVTK | EH-domain containing protein 2 |
| TVDLLAGLGAERPETANTAQS*PYK | Epilepsy holoprosencephaly candidate-1 protein |
| YADSPGASS*PEQPK | ETS-related transcription factor Elf-1 |
| SPS*LSPK | ETS-domain protein Elk-3 |
| APVSSTESVIQSNTPT*PPPSQPLNETAEEESR | Echinoderm microtubule-associated protein-like 4 |
| SS*PELLPSGVTDENEVTTAVTEK | Epidermal growth factor receptor substrate 15 |
| ASSLSESS*PPK | Epithelial protein lost in neoplasm |
| NSPDECS*VAK | Transcriptional regulator ERG |
| AEPASPDS*PKGSS*ETETEPPVALAPGPAPTR | Steroid hormone receptor ERR1 |
| S*NS*VEKPVSSILSR | Ena/vasodilator stimulated phosphoprotein-like protein |
| SAS*PTVPR | Envoplakin |
| ESSIIAPAPAEDVDT*PPR | Enhancer of zeste homolog 2 |
| S*PILEEK | Fetal Alzheimer antigen |
| ADEASELACPT*PK | Fatty acid synthase |
| SGTNS*PPPPFSDWGR | F-box only protein 4 |
| S*LEGGGCPAR | FH1/FH2 domains-containing protein |
| NNEES*PTATVAEQGEDITSK | FK506-binding protein 5 |
| NAEAVLQS*PGLSGK | Flightless-I protein homolog |
| AFGPGLQGGSAGS*PAR | Filamin A |
| CSGPGLS*PGMVR | Filamin A |
| QEPLEEDS*PSSSSAGLDK | Fos-related antigen 2 |
| S*PPAPGLQPMR | Fos-related antigen 2 |
| HTLGDS*DNES | Ferritin heavy chain |
| MGAPESGLAEYLFDKHTLGDS*DNES | Ferritin heavy chain |
| LLSSEPLDLISVPFGNSSPSDIDVPKPGS*PEPQVSGLAANR | Forkhead box protein M1 |
| LEPAS*PPEDTSAEVSR | General transcription factor II-I repeat domain-containing |
| | protein 1 |
| SSS*PAPADIAQTVQEDLR | Ras-GTPase-activating protein binding protein 1 |
| AASSSSPGS*PVASSPSR | Golgi-specific brefeldin A-resistance guanine nucleotide |
| | exchange factor 1 |
| VLSGNCNHQEGTS*S*DDELPSAEMIDFQK | GC-rich sequence DNA-binding factor |
| APGGESLLGPGPS*PPSALTPGLGAEAGGGFPGGAEPGNGLKPR | GC-rich sequence DNA-binding factor homolog |
| MADHLEGLS*S*DDEETSTDITNFNLEK | GC-rich sequence DNA-binding factor homolog |
| ISVIFS*LEELK | Gamma-tubulin complex component 6 |
| SQSDLDDQHDYDSVAS*DEDTDQEPLR | ARF GTPase-activating protein GIT1 |
| VPS*VESLFR | Golgi autoantigen, golgin subfamily A member 4 |
| ALQS*PK | General transcription factor II-I |
| S*PGSNSK | General transcription factor II-I |
| SPS*WYGIPR | General transcription factor II-I |
| VPQALNFS*PEESDSTFSK | G2 and S phase expressed protein 1 |
| AGGSAALS*PSK | Histone H1x |
| QNPQS*PPQDSSVTSK | Histone deacetylase 6 |
| AGDLLEDS*PK | Hepatoma-derived growth factor |
| GNAEGS*S*DEEGKLVIDEPAK | Hepatoma-derived growth factor |
| NST*PSEPGSGR | Hepatoma-derived growth factor |
| NSTPS*EPGSGR | Hepatoma-derived growth factor |
| S*PSPVQR | Potential helicase with zinc-finger domain |
| EDLPAENGETKTEES*PASDEAGEK | Nonhistone chromosomal protein HMG-14 |
| QAEVANQET*KEDLPAENGETKTEESPAS*DEAGEK | Nonhistone chromosomal protein HMG-14 |
| EESEES*EAEPVQR | HIRA-interacting protein 3 |
| ESEQES*EEEILAQK | HIRA-interacting protein 3 |
| EVS*DSEAGGGPQGER | HIRA-interacting protein 3 |
| FNSESES*GSEASSPDYFGPPAK | HIRA-interacting protein 3 |
| NGVAAEVS*PAKEENPR | HIRA-interacting protein 3 |
| SLKES*EQES*EEEILAQK | HIRA-interacting protein 3 |
| KLEKEEEEGIS*QES*S*EEEQ | High mobility group protein HMG-I/HMG-Y |
| KSLDS*DES*EDEEDDYQQK | 28 kDa heat- and acid-stable phosphoprotein |
| SLDS*DESEDEEDDYQQK | 28 kDa heat- and acid-stable phosphoprotein |
| ALSSAVQASPTS*PGGSPSSPSSGQR | Zinc finger protein HRX |
| NSSTPGLQVPVS*PTVPIQNQK | Zinc finger protein HRX |
| NTPSMQALGES*PESSSSELLNLGEGLGLDSNR | Zinc finger protein HRX |
| SPT*VPSQNPSR | Zinc finger protein HRX |
| TPSYS*PTQR | Zinc finger protein HRX |
| QLSS*GVSEIR | Heat shock 27 kDa protein |
| FELTGIPPAPRGVPQIEVT*FDIDANGILNVSAVDK | Heat shock cognate 71 kDa protein |
| IEDVGS*DEEDDS*GKDK | Heat shock protein HSP 90-beta |
| VKEEPPS*PPQS*PR | Heat shock factor protein 1 |
| EGITGPPADSSKPIGPDDAIDALSSDFTCGS*PTAAGK | Calpain inhibitor |
| VSEEQTQPPS*PAGAGMSTAMGR | Gamma-interferon-inducible protein Ifi-16 |
| IEPIPGES*PK | Translation initiation factor IF-2 |
| INS*SGESGDESDEFLQSR | Translation initiation factor IF-2 |
| INSSGES*GDESDEFLQSR | Translation initiation factor IF-2 |
| INSSGESGDES*DEFLQSR | Translation initiation factor IF-2 |
| QS*FDDNDS*EELEDKDSK | Translation initiation factor IF-2 |
| VEMYS*GSDDDDDFNK | Translation initiation factor IF-2 |
| WDGS*EEDEDNSK | Translation initiation factor IF-2 |
| GIPLATGDTS*PEPELLPGAPLPPPKEVINGNIK | Eukaryotic translation initiation factor 3 subunit 4 |
| QLT*PPEGSSK | Eukaryotic translation initiation factor 3 subunit 8 |
| QNPEQS*ADEDAEK | Eukaryotic translation initiation factor 3 subunit 8 |
| AQAVS*EDAGGNEGR | Eukaryotic translation initiation factor 3 subunit 9 |
| TEPAAEAEAASGPSES*PS*PPAAEELPGSHAEPPVPAQGEAPGEQAR | Eukaryotic translation initiation factor 3 subunit 9 |
| TEPAAEAEAASGPSESPS*PPAAEELPGSHAEPPVPAQGEAPGEQAR | Eukaryotic translation initiation factor 3 subunit 9 |
| S*PPYTAFLGNLPYDVTEESIK | Eukaryotic translation initiation factor 4B |
| SQSS*DTEQQSPTSGGGK | Eukaryotic translation initiation factor 4B |
| SQSSDTEQQS*PTSGGGK | Eukaryotic translation initiation factor 4B |
| AAS*LTEDR | Eukaryotic translation initiation factor 4 gamma |
| EAALPPVS*PLK | Eukaryotic translation initiation factor 4 gamma |
| DSSKGEDS*AEETEAKPAVVAPAPVVEAVSTPSAAFPSDATAEQGPILTK | Interleukin enhancer-binding factor 3 |
| GSSEQAES*DNMDVPPEDDSK | Interleukin enhancer-binding factor 3 |
| LFPDT*PLALDANK | Interleukin enhancer-binding factor 3 |
| IQEQESS*GEEDSDLSPEER | Protein phosphatase inhibitor 2 |
| ALQS*PALGLR | Ras GTPase-activating-like protein IQGAP1 |
| S*PGEYINIDFGEPGAR | Insulin receptor substrate-2 |
| SNT*PESIAETPPAR | Insulin receptor substrate-2 |
| SSEGGVGVGPGGGDEPPTS*PR | Insulin receptor substrate-2 |
| VAS*PTSGVK | Insulin receptor substrate-2 |
| S*PGPLPGAR | Insulin gene enhancer protein ISL-2 |
| SAFTPATATGSSPS*PVLGQGEK | Intersectin 1 |
| LFSSSSS*PPPAK | C-jun-amino-terminal kinase interacting protein 3 |
| DATPPVS*PINMEDQER | Transcription factor jun-B |
| LAALKDEPQTVPDVPSFGES*PPLSPIDMDTQER | Transcription factor jun-D |
| SYTS*GPGSR | Keratin, type II cytoskeletal 8 |
| ASYDVSDSGQLEHVQPWS*V | 6-phosphofructokinase, type C |
| S*PPLPAVIR | Protein KIAA0852 |
| SVAVS*DEEEVEEEAER | Protein KIAA0852 |
| VYYS*PPVAR | Protein KIAA0889 |
| IQPAGNTS*PR | Casein kinase I, epsilon isoform |
| MSDTGS*PGMQR | Kinesin-like protein KIF1B |
| SGLS*LEELR | Kinesin-like protein KIF1B |
| SVS*PSPVPLLFQPDQNAPPIR | Kinesin-like protein KIF23 |
| IQAAAST*PTNATAASDANTGDR | Glycogen synthase kinase-3 beta |
| EDSGSSS*PPGVFLEK | Protein KIAA1688 |
| AQSLVIS*PPAPSPR | Antigen KI-67 |
| IPCES*PPLEVVDTTASTK | Antigen KI-67 |
| MPCESS*PPESADTPTSTR | Antigen KI-67 |
| TPVQYSQQQNS*PQK | Antigen KI-67 |
| ASS*LNFLNK | Kinesin light chain 2 |
| QSST*PSAPELGQQPDVNISEWK | Phosphorylase B kinase beta regulatory chain |
| NLIDSMDQSAFAGFS*FVNPK | Protein kinase C, delta type |
| GDGGSTTGLSAT*PPASLPGSLTNVK | B-Raf proto-oncogene serine/threonine-protein kinase |
| SAS*EPSLNR | B-Raf proto-oncogene serine/threonine-protein kinase |
| TEGDEEAEEEQEENLEAS*GDYK | ATP-dependent DNA helicase II, 70 kDa subunit |
| LRLS*PS*PTSQR | Lamin A/C |
| SGAQASSTPLS*PTR | Lamin A/C |
| SYLLGNSS*PR | Lamin A/C |
| SADGS*APAGEGEGVTLQR | Large neutral amino acids transporter small subunit 1 |
| LQAGEYVS*LGK | Long-chain-fatty-acid-CoA ligase 3 |
| SS*PPSIAPLALDSADLS*EEK | Ligatin |
| S*PPPR | LIM-only protein 6 |
| DGVLTLANNVT*PAK | Microtubule-associated protein 4 |
| DMES*PTK | Microtubule-associated protein 4 |
| DMS*PLSETEMALGKDVT*PPPETEVVLIK | Microtubule-associated protein 4 |
| DVT*PPPETEVVLIK | Microtubule-associated protein 4 |
| S*QESGYYDR | Matrin 3 |
| S*YSPDGK | Matrin 3 |
| SYS*PDGK | Matrin 3 |
| SYS*PDGKES*PSDK | Matrin 3 |
| SAGAPASVSGQDADGSTS*PR | Megakaryocyte-associated tyrosine-protein kinase |
| AIPELDAYEAEGLALDDEDVEELT*ASQR | DNA replication licensing factor MCM2 |
| GNDPLTSS*PGR | DNA replication licensing factor MCM2 |
| RTDALTS*S*PGR | DNA replication licensing factor MCM2 |
| TDALTSS*PGR | DNA replication licensing factor MCM2 |
| DGDSYDPYDFSDT*EEEMPQVHT*PK | DNA replication licensing factor MCM3 |
| IAEPS*VCGR | DNA replication licensing factor MCM4 |
| AEENTDQAS*PQEDYAGFER | Midasin |
| NGGEDT*DNEEGEEENPLEIK | Midasin |
| AETSEGSGSAPAVPEASAS*PK | Methyl-CpG-binding protein 2 |
| NSVSPGLPQRPASAGAMLGGDLNS*ANGACPSPVGNGYVSAR | Myocyte-specific enhancer factor 2D |
| IVEPEVVGES*DS*EVEGDAWR | Microfibrillar-associated protein 1 |
| IVEPEVVGESDS*EVEGDAWR | Microfibrillar-associated protein 1 |
| MEREDS*S*EEEEEEIDDEEIER | Microfibrillar-associated protein 1 |
| SLAALDALNT*DDENDEEEYEAWK | Microfibrillar-associated protein 1 |
| AQETEAAPSQAPADEPEPES*AAAQSQENQDTRPK | Melanoma-associated antigen D2 |
| LQSS*QEPEAPPPR | Melanoma-associated antigen D2 |
| GAGATSGS*PPAGRN | Methylated-DNA-protein-cysteine methyltransferase |
| SPLVTGS*PK | Probable tumor suppressor protein MN1 |
| LNQPGT*PTR | Dual specificity mitogen-activated protein kinase kinase 2 |
| GVDFES*S*EDDDDDPFMNTSSLR | Double-strand break repair protein MRE11A |
| GVDFES*SEDDDDDPFMNTSSLR | Double-strand break repair protein MRE11A |
| TLHT*CLELLR | Double-strand break repair protein MRE11A |
| IHNVGS*PLK | DNA mismatch repair protein MSH6 |
| SEEDNEIES*EEEVQPK | DNA mismatch repair protein MSH6 |
| VIS*DS*ES*DIGGSDVEFKPDTK | DNA mismatch repair protein MSH6 |
| VIS*DSESDIGGS*DVEFKPDTK | DNA mismatch repair protein MSH6 |
| VAPVINNGS*PTILGK | Metastasis-associated protein MTA1 |
| AES*FMFRT*WGADVINMTTVPEVVLAK | 5′-methylthioadenosine phosphorylase |
| MDS*ALTARDR | Myosin Ic |
| GELIPIS*PSTEVGGSGIGTPPSVLK | Myb-related protein B |
| KFELLPT*PPLS*PSR | N-myc proto-oncogene protein |
| GPVGTVS*EAQLAR | Myoferlin |
| FSS*PIVK | Nuclear pore complex protein Nup153 |
| S*PGSTPTTPTSSQAPQK | Nuclear pore complex protein Nup214 |
| SPGSTPTT*PTSSQAPQK | Nuclear pore complex protein Nup214 |
| QGGS*PDEPDSK | Neighbor of A-kinase anchoring protein 95 |
| DGAVNGPSVVGDQT*PIEPQTSIER | Nuclear autoantigenic sperm protein |
| LVPS*QEETK | Nuclear autoantigenic sperm protein |
| AVS*LDSPVSVGSSPPVK | Nuclear receptor coactivator 3 |
| QSNSGAT*K | Nuclear receptor coactivator 6 |
| HEAPSS*PISGQPCGDDQNAS*PSK | Nuclear receptor co-repressor 1 |
| S*PGSISYLPSFFTK | Nuclear receptor co-repressor 1 |
| VS*PENLVDK | Nuclear receptor co-repressor 1 |
| YETPSDAIEVIS*PASSPAPPQEK | Nuclear receptor co-repressor 1 |
| S*PGNTSQPPAFFSK | Nuclear receptor co-repressor 2 |
| SGLEPASS*PSK | Nuclear receptor co-repressor 2 |
| SRT*AS*GSSVTSLDGTR | NDRG1 protein |
| TAS*GSSVTSLDGTR | NDRG1 protein |
| TASGSSVTS*LDGTR | NDRG1 protein |
| YFVQGMGYMPSAS*MTR | NDRG1 protein |
| GSEGYLAATYPTVGQTS*PR | Neurofibromin |
| SNSGLATYS*PPMGPVSER | Neurofibromin |
| SVEDEMDS*PGEEPFYTGQGR | Nuclear factor 1 A-type |
| DAEQSGS*PR | Nuclear factor 1 C-type |
| SGSMEEDVDTSPGGDYYTSPSS*PTSSSR | Nuclear factor 1 C-type |
| SPFNSPS*PQDSPR | Nuclear factor 1 C-type |
| TEMDKS*PFNSPS*PQDSPR | Nuclear factor 1 C-type |
| AAPEASS*PPAS*PLQHLLPGK | Niban-like protein |
| GLLAQGLRPES*PPPAGPLLNGAPAGESPQPK | Niban-like protein |
| GGLS*PANDTGAK | Glycylpeptide N-tetradecanoyltransferase 1 |
| EAAAGIQWSEEETEDEEEEKEVT*PESGPPK | Proliferating-cell nucleolar antigen p120 |
| GGSISVQVNSIKFDS*E | Nucleolar phosphoprotein p130 |
| GSS*PSR | Orphan nuclear receptor NR1D1 |
| LLDEYNVTPS*PPGTVLTSALSPVICGPNR | Neurogenic locus notch homolog protein 2 precursor |
| TPSLALT*PPQAEQEVDVLDVNVR | Neurogenic locus notch homolog protein 2 precursor |
| DSENLAS*PSEYPENGER | Nuclear pore complex protein Nup98-Nup96 precursor |
| EVEEDS*EDEEMSEDEEDDSSGEEVVIPQKK | Nucleolin |
| KEDS*DEEEDDDSEEDEEDDEDEDEDEDEIEPAAM | Nucleolin |
| KEDSDEEEDDDS*EEDEEDDEDEDEDEDEIEPAAM | Nucleolin |
| VVVS*PTK | Nucleolin |
| ATVT*PS*PVKGK | Nuclear ubiquitous casein and cyclin-dependent kinases |
| | substrate |
| DSGSDEDFLMEDDDDS*DYGSSK | Nuclear ubiquitous casein and cyclin-dependent kinases |
| | substrate |
| NSQEDS*EDS*EDKDVK | Nuclear ubiquitous casein and cyclin-dependent kinases |
| | substrate |
| TPS*PKEEDEEPES*PPEK | Nuclear ubiquitous casein and cyclin-dependent kinases |
| | substrate |
| TS*TSPPPEKSGDEGSEDEAPSGED | Nuclear ubiquitous casein and cyclin-dependent kinases |
| | substrate |
| TSTS*PPPEK | Nuclear ubiquitous casein and cyclin-dependent kinases |
| | substrate |
| TSTSPPPEKS*GDEGSEDEAPSGED | Nuclear ubiquitous casein and cyclin-dependent kinases |
| | substrate |
| VVDYSQFQES*DDADEDYGR | Nuclear ubiquitous casein and cyclin-dependent kinases |
| | substrate |
| YGMGTS*VER | Pyruvate dehydrogenase E1 component alpha subunit, |
| | somatic form, mitochondrial precursor |
| SFSLASSSNS*PISQR | Oxysterol binding protein-related protein 11 |
| MLAES*DES*GDEESVSQTDKTELQNTLR | Oxysterol-binding protein 1 |
| SKELVSSSSSGSDS*DS*EVDK | Activated RNA polymerase II transcriptional |
| | coactivator p15 |
| EQLSAQELMESGLQIQKS*PEPEVLSTQEDLFDQSNK | Tumor suppressor p53-binding protein 1 |
| IDEDGENTQIEDTEPMS*PVLNSK | Tumor suppressor p53-binding protein 1 |
| LMLSTSEYSQS*PK | Tumor suppressor p53-binding protein 1 |
| MVIQGPSS*PQGEAMVTDVLEDQK | Tumor suppressor p53-binding protein 1 |
| NGSTAVAESVAS*PQK | Tumor suppressor p53-binding protein 1 |
| NS*PEDLGLSLTGDSCK | Tumor suppressor p53-binding protein 1 |
| S*PEPEVLSTQEDLFDQSNK | Tumor suppressor p53-binding protein 1 |
| SEDPPTT*PIR | Tumor suppressor p53-binding protein 1 |
| SGTAETEPVEQDSS*QPSLPLVR | Tumor suppressor p53-binding protein 1 |
| STPFIVPSS*PTEQEGR | Tumor suppressor p53-binding protein 1 |
| TVSS*DGCSTPSR | Tumor suppressor p53-binding protein 1 |
| VDVSCEPLEGVEKCS*DSQSWEDIAPEIEPCAENR | Tumor suppressor p53-binding protein 1 |
| LGFSLT*PSK | Coilin |
| CSVS*LSNVEAR | Cytosolic phospholipase A2 |
| TSPLNSSGSS*QGR | Poly(A) polymerase alpha |
| HYGITSPISLAS*PEEIDHIYTQK | Poly(A) polymerase gamma |
| VMTIPYQPMPASS*PVICAGGQDR | Poly(rC)-binding protein 1 |
| KVMDS*DEDDDY | Programmed cell death protein 5 |
| IDT*PPACTEESIATPSEIK | Pre-mRNA cleavage complex II protein Pcf11 |
| S*PSLSSK | Protocadherin 7 precursor |
| DGELPVEDDIDLS*DVELDDLGKDEL | Protein disulfide isomerase A6 precursor |
| ANS*FVGTAQYVSPELLTEK | 3-phosphoinositide dependent protein kinase-1 |
| AFT*PFSGPK | Xaa-Pro dipeptidase |
| AS*QEEQIAR | Periplakin |
| EGEEPTVYS*DEEEPKDESAR | Membrane associated progesterone receptor component 1 |
| GDQPAASGDS*DDDEPPPLPR | Membrane associated progesterone receptor component 1 |
| S*LGDEGLNR | 1-phosphatidylinositol-4,5-bisphosphate phosphodiesterase |
| | beta 3 |
| ELSESVQQQSTPVPLIS*PK | Protein kinase C binding protein 1 |
| STS*PASEK | Protein kinase C binding protein 1 |
| TGQAGS*LSGS*PKPFSPQLSAPITTK | Protein kinase C binding protein 1 |
| TDVSNFDEEFTGEAPTLS*PPR | Protein kinase C-like 1 |
| AS*SLGEIDESSELR | Protein kinase C-like 2 |
| TST*FCGTPEFLAPEVLTETSYTR | Protein kinase C-like 2 |
| AGGLDWPEATEVS*PSR | Plakophilin 3 |
| AQLEPVAS*PAK | Plectin 1 |
| GYYS*PYSVSGSGSTAGSR | Plectin 1 |
| GYYSPYSVSGSGST*AGSR | Plectin 1 |
| SDEGQLS*PATR | Plectin 1 |
| SSS*VGSSSSYPISPAVSR | Plectin 1 |
| T*QLASWSDPTEETGPVAGILDTETLEK | Plectin 1 |
| INPPSSGGTSSS*PIK | POU domain, class 2, transcription factor 1 |
| SAVCIADPLPTPS*QEK | Ribonucleases P/MRP protein subunit POP1 |
| VQAYEEPSVASS*PNGK | Ribonucleases P/MRP protein subunit POP1 |
| LTFDSSFS*PNTGK | Voltage-dependent anion-selective channel protein 1 |
| GLCIKS*REIFLS*QPILLELEAPLK | Serine/threonine protein phosphatase PP1-beta catalytic |
| | subunit |
| QVPDS*AATATAYLCGVK | Alkaline phosphatase, intestinal precursor |
| LPST*SDDCPAIGTPLR | Peroxisome proliferator-activated receptor binding protein |
| LPSTSDDCPAIGT*PLR | Peroxisome proliferator-activated receptor binding protein |
| MSS*LLER | Peroxisome proliferator-activated receptor binding protein |
| NSSQSGGKPGSS*PITK | Peroxisome proliferator-activated receptor binding protein |
| SQT*PPGVATPPIPK | Peroxisome proliferator-activated receptor binding protein |
| DAS*PINRWS*PTR | Serine/threonine-protein kinase PRP4 homolog |
| EQPEMEDANS*EKS*INEENGEVSEDQSQNK | Serine/threonine-protein kinase PRP4 homolog |
| S*LS*PKPR | Serine/threonine-protein kinase PRP4 homolog |
| S*PIINESR | Serine/threonine-protein kinase PRP4 homolog |
| S*PVDLR | Serine/threonine-protein kinase PRP4 homolog |
| S*RS*PLLNDR | Serine/threonine-protein kinase PRP4 homolog |
| SINEENGEVS*EDQSQNK | Serine/threonine-protein kinase PRP4 homolog |
| SINEENGEVSEDQS*QNK | Serine/threonine-protein kinase PRP4 homolog |
| SPS*PDDILER | Serine/threonine-protein kinase PRP4 homolog |
| TLS*PGR | Serine/threonine-protein kinase PRP4 homolog |
| TRS*PS*PDDILER | Serine/threonine-protein kinase PRP4 homolog |
| YLAEDSNMSVPSEPSS*PQSSTR | Serine/threonine-protein kinase PRP4 homolog |
| YLAEDSNMSVPSEPSSPQSST*R | Serine/threonine-protein kinase PRP4 homolog |
| TAS*PPALPK | PR-domain zinc finger protein 2 |
| T*SQLLPCSPSK | PR-domain zinc finger protein 14 |
| LTPLPEDNS*MNVDQDGDPSDR | DNA-dependent protein kinase catalytic subunit |
| ESLKEEDES*DDDNM | Proteasome subunit alpha type 3 |
| ITS*PLMEPSSIEK | Proteasome subunit alpha type 5 |
| TPEAS*PEPK | 26S proteasome non-ATPase regulatory subunit 1 |
| TSSAFVGKT*PEAS*PEPK | 26S proteasome non-ATPase regulatory subunit 1 |
| TVGT*PIASVPGSTNTGTVPGSEK | 26S proteasome non-ATPase regulatory subunit 1 |
| EKLQEEGGGS*DEEETGS*PSEDGMQSAR | Periodic tryptophan protein 1 homolog |
| SGS*S*SPDSEITELKFPSINHD | CTP synthase |
| SGSSS*PDSEITELK | CTP synthase |
| NDLQDTEIS*PR | Postreplication repair protein RAD18 |
| NHLLQFALES*PAK | Postreplication repair protein RAD18 |
| GFGSEEGS*R | RNA-binding protein 8A |
| NGTGQSS*DSEDLPVLDNSSK | Retinoblastoma-binding protein 1 |
| QGPVS*PGPAPPPSFIMSYK | Retinoblastoma-binding protein 2 |
| VVSSVSSS*PR | Retinoblastoma-binding protein 2 |
| VSS*PVFGATSSIK | Retinoblastoma-binding protein 8 |
| VVNPLIGLLGEYGGDSDYEEEEEEEQT*PPPQPR | RNA-binding protein 6 |
| SFS*SPENFQR | Putative RNA-binding protein 7 |
| GLVAAYSGES*DSEEEQER | RNA-binding protein 10 |
| GLVAAYSGESDS*EEEQER | RNA-binding protein 10 |
| LGGSGGSNGS*SSGK | Putative RNA-binding protein 15 |
| LHS*YSS*PSTK | Putative RNA-binding protein 15 |
| SLS*PGGAALGYR | Putative RNA-binding protein 15 |
| AVVS*PPK | Ran-binding protein 2 |
| LNQSGTS*VGTDEESDVTQEEER | Ran-binding protein 2 |
| SALS*PSKS*PAK | Ran-binding protein 2 |
| T*SPENVQDR | Ran-binding protein 2 |
| YIASVQGSTPS*PR | Ran-binding protein 2 |
| YSLS*PSK | Ran-binding protein 2 |
| S*PPADAIPK | Regulator of chromosome condensation |
| SIS*ADDDLQESSR | RD protein |
| NLDNVS*PK | Double-stranded RNA-specific editase 1 |
| VDDDS*LGEFPVTNSR | Zinc-finger protein ubi-d4 |
| ATS*PLCTSTASMVSSS*PSTPSNIPQKPSQPAAK | Restin |
| TASESISNLSEAGS*IK | Restin |
| ESVS*PEDSEK | Activator 1 140 kDa subunit |
| ASETVSEAS*PGSTASQTGVPTQVVQQVQGTQQR | MHC class II regulatory factor RFX1 |
| ILDPNTGEPAPVLSSPPPADVST*FLAFPSPEKLLR | Ran GTPase-activating protein 1 |
| KILDPNTGEPAPVLSS*PPPADVSTFLAFPS*PEK | Ran GTPase-activating protein 1 |
| VEAKEESEES*DEDMGFGLFD | 60S acidic ribosomal protein P0 |
| NMGGPYGGGNYGPGGSGGS*GGYGGR | Heterogeneous nuclear ribonucleoproteins A2/B1 |
| DDEKEAEEGEDDRDS*ANGEDDS | Heterogeneous nuclear ribonucleoproteins C1/C2 |
| EAEEGEDDRDS*ANGEDDS | Heterogeneous nuclear ribonucleoproteins C1/C2 |
| MESEGGADDS*AEEGDLLDDDDNEDRGDDQLELIK | Heterogeneous nuclear ribonucleoproteins C1/C2 |
| NEEDEGHSNSS*PR | Heterogeneous nuclear ribonucleoprotein D0 |
| ATENDIYNFFS*PLNPVR | Heterogeneous nuclear ribonucleoprotein F |
| GFAFVTFES*PADAK | Heterogeneous nuclear ribonucleoprotein G |
| GLPWSCS*ADEVQR | Heterogeneous nuclear ribonucleoprotein H |
| DYDDMS*PR | Heterogeneous nuclear ribonucleoprotein K |
| IIPTLEEGLQLPS*PTATSQLPLESDAVECLNYQHYK | Heterogeneous nuclear ribonucleoprotein K |
| MET*EQPEETFPNTETNGEFGK | Heterogeneous nuclear ribonucleoprotein K |
| IFVGGLS*PDTPEEK | Heterogeneous nuclear ribonucleoprotein UP2 |
| IFVGGLSPDT*PEEK | Heterogeneous nuclear ribonucleoprotein UP2 |
| YSPTSPTYS*PTSPVYTPTSPK | DNA-directed RNA polymerase II largest subunit |
| YSPTSPTYSPTS*PK | DNA-directed RNA polymerase II largest subunit |
| AEGS*PNQGK | Ribosome-binding protein 1 |
| NTDVAQS*PEAPK | Ribosome-binding protein 1 |
| ANS*GGVDLDSSGEFASIEK | RAS-responsive element binding protein 1 |
| DEILPTT*PISEQK | 40S ribosomal protein S3 |
| RFT*PPSTALS*PGK | Runt-related transcription factor 1 |
| ISS*PTETER | S100 calcium-binding protein A14 |
| LIHEQEQQSSS* | Putative S100 calcium-binding protein MGC17528 |
| ASPGTPLS*PGSLR | Solute carrier family 21 member 12 |
| NCAS*PSSAGQLILPECMK | Protein transport protein Sec24C |
| AEEPPSQLDQDTQVQDMDEGS*DDEEEGQK | Splicing factor 3 subunit 1 |
| GGDSIGETPT*PGASK | Splicing factor 3B subunit 1 |
| WDETPAS*QMGGSTPVLT*PGK | Splicing factor 3B subunit 1 |
| WDETPASQMGGST*PVLTPGK | Splicing factor 3B subunit 1 |
| SS*LGQSASETEEDTVSVSK | Splicing factor 3B subunit 2 |
| SSLGQS*ASETEEDTVSVSK | Splicing factor 3B subunit 2 |
| SSLGQSAS*ETEEDTVSVSK | Splicing factor 3B subunit 2 |
| AKS*PT*PDGSER | Putative splicing factor YT521 |
| GIS*PIVFDR | Putative splicing factor YT521 |
| SEASDSGS*ESVSFTDGSVR | Putative splicing factor YT521 |
| SGS*SASESYAGSEK | Putative splicing factor YT521 |
| SGSSAS*ESYAGSEK | Putative splicing factor YT521 |
| SGSSASESYAGS*EK | Putative splicing factor YT521 |
| SPT*PDGSER | Putative splicing factor YT521 |
| GSS*FQSGR | Exocyst complex component Sec5 |
| ESIS*PQPADSACSSPAPSTGK | Sentrin-specific protease 6 |
| LNYSDES*PEAGK | Sentrin-specific protease 6 |
| S*RS*PPPVSK | Splicing factor, arginine/serine-rich 2 |
| SPPKS*PEEEGAVSS | Splicing factor, arginine/serine-rich 2 |
| TS*PDTLR | Splicing factor, arginine/serine-rich 2 |
| SPAS*VDR | Splicing factor, arginine/serine-rich 5 |
| SVS*RS*PVPEK | Splicing factor, arginine/serine-rich 5 |
| ARS*VS*PPPK | Splicing factor, arginine/serine-rich 6 |
| S*NSPLPVPPSK | Splicing factor, arginine/serine-rich 6 |
| S*VS*PPPKR | Splicing factor, arginine/serine-rich 6 |
| SVS*PPPK | Splicing factor, arginine/serine-rich 6 |
| S*RSPSGS*PR | Splicing factor, arginine/serine-rich 7 |
| SAS*PERMD | Splicing factor, arginine/serine-rich 7 |
| SPS*GSPR | Splicing factor, arginine/serine-rich 7 |
| SPS*PK | Splicing factor, arginine/serine-rich 7 |
| YFQS*PSR | Splicing factor, arginine/serine-rich 7 |
| ARS*QSVS*PSK | Splicing factor, arginine/serine-rich 8 |
| S*PGASR | Splicing factor, arginine/serine-rich 8 |
| SQSVS*PSK | Splicing factor, arginine/serine-rich 8 |
| STS*YGYSR | Splicing factor, arginine/serine-rich 9 |
| SRT*PSASNDDQQE | Small glutamine-rich tetratricopeptide repeat-containing |
| | protein |
| ASS*LEDLVLK | Helicase SKI2W |
| GDTVSAS*PCSAPLAR | Helicase SKI2W |
| KACYS*K | Semaphorin 5A precursor |
| VQGLLENGDSVTS*PEK | SmcX protein |
| GPS*PSPVGSPASVAQSR | SWI/SNF-related, matrix-associated, actin-dependent |
| | regulator of chromatin subfamily F member 1 |
| NPQMPQYSSPQPGSALS*PR | SWI/SNF-related, matrix-associated, actin-dependent |
| | regulator of chromatin subfamily F member 1 |
| VSS*PAPMEGGEEEEELLGPK | SWI/SNF-related, matrix-associated, actin-dependent |
| | regulator of chromatin subfamily F member 1 |
| TTS*PEPQESPTLPSTEGQVVNK | Smoothelin |
| AEENAEGGESALGPDGEPIDESSQMS*DLPVK | Possible global transcription activator SNF2L2 |
| EVDYSDS*LTEK | Possible global transcription activator SNF2L4 |
| IPDPDS*DDVSEVDAR | Possible global transcription activator SNF2L4 |
| VAELTSLS*DEDSGK | Zinc finger protein SNAI1 |
| AVNTQALS*GAGILR | Sorting nexin 2 |
| ESDQTLAALLS*PK | SON protein |
| S*AASPVVSSMPER | SON protein |
| S*FSISPVR | SON protein |
| S*PDPYR | SON protein |
| SAAS*PVVSSMPER | SON protein |
| SFSIS*PVR | SON protein |
| SVESTS*PEPSK | SON protein |
| YDVDLSLTTQDTEHDMVISTSPSGGS*EADIEGPLPAK | SON protein |
| IPESETESTASAPNS*PR | Son of sevenless protein homolog 1 |
| TSISDPPES*PPLLPPR | Son of sevenless protein homolog 1 |
| SSSTGSSSSTGGGGQESQPS*PLALLAATCSR | Transcription factor Sp1 |
| ENNVSQPASSSSSSSSSNNGSASPT*K | Transcription factor Sp4 |
| SGS*DAGEARPPTPAS*PR | Signal-induced proliferation-associated protein 1 |
| CTELNQAWSS*LGK | Spectrin alpha chain, brain |
| GEQVS*QNGLPAEQGSPR | Spectrin beta chain, brain 1 |
| GEQVSQNGLPAEQGS*PR | Spectrin beta chain, brain 1 |
| TSSKESS*PIPS*PTSDR | Spectrin beta chain, brain 1 |
| S*PQTLAPVGEDAMK | Symplekin |
| IEIIQPLLDMAAGTSNAAPVAENVTNNEGS*PPPPVK | CTD-binding SR-like protein RA4 |
| TT*PTQPSEQK | CTD-binding SR-like protein RA4 |
| AKTQT*PPVS*PAPQPTEER | Src substrate cortactin |
| LPSS*PVYEDAASFK | Src substrate cortactin |
| TQT*PPVSPAPQPTEER | Src substrate cortactin |
| VGGS*DEEASGIPSR | Suppressor of SWI4 1 homolog |
| EGMNPSYDEYADS*DEDQHDAYLER | Structure-specific recognition protein 1 |
| SKEFVSS*DESSS*GENK | Structure-specific recognition protein 1 |
| GTDAT*NPPEGPQDR | Stanniocalcin 2 precursor |
| QVAEQGGDLS*PAANR | serine/threonine protein kinase 10 |
| NLEQILNGGES*PK | Striatin 3 |
| EYIPGQPPLSQSS*DSS*PTRNSEPAGLETPEAK | Bifunctional aminoacyl-tRNA synthetase |
| NQGGGLSSS*GAGEGQGPK | Bifunctional aminoacyl-tRNA synthetase |
| NSEPAGLET*PEAK | Bifunctional aminoacyl-tRNA synthetase |
| LLS*SNEDDANILSSPTDR | Thyroid hormone receptor-associated protein |
| | complex 100 kDa component |
| LLSS*NEDDANILSSPTDR | Thyroid hormone receptor-associated protein |
| | complex 100 kDa component |
| AS*AVSELSPR | Thyroid hormone receptor-associated protein complex |
| | 150 kDa component |
| ASAVSELS*PR | Thyroid hormone receptor-associated protein complex |
| | 150 kDa component |
| AVQEKSS*S*PPPR | Thyroid hormone receptor-associated protein complex |
| | 150 kDa component |
| EQTFSGGTS*QDTK | Thyroid hormone receptor-associated protein |
| | complex 150 kDa component |
| FSGEEGEIEDDES*GTENR | Thyroid hormone receptor-associated protein |
| | complex 150 kDa component |
| GSFS*DTGLGDGK | Thyroid hormone receptor-associated protein |
| | complex 150 kDa component |
| IDIS*PSTFR | Thyroid hormone receptor-associated protein complex |
| | 150 kDa component |
| S*PPSTGSTYGSSQK | Thyroid hormone receptor-associated protein complex |
| | 150 kDa component |
| SPPST*GSTYGSSQK | Thyroid hormone receptor-associated protein complex |
| | 150 kDa component |
| SSS*PPPR | Thyroid hormone receptor-associated protein complex |
| | 150 kDa component |
| SSSS*SS*QSSHSYK | Thyroid hormone receptor-associated protein complex |
| | 150 kDa component |
| SNDS*TDGEPEEK | TBP-associated factor 172 |
| GAGGPAS*AQGSVK | Thyroid hormone receptor-associated protein complex |
| | 240 kDa component |
| LLEPPVLTLDPNDENLILEIPDEKEEATSNS*PSK | Transcription initiation factor TFIID 250 kDa subunit |
| QEAGDS*PPPAPGTPK | Transcription initiation factor TFIID 70 kDa subunit |
| AS*PEPPGPESSSR | 182 kDa tankyrase 1-binding protein |
| HNGS*LS*PGLEAR | 182 kDa tankyrase 1-binding protein |
| VPSS*DEEVVEEPQSR | 182 kDa tankyrase 1-binding protein |
| VSGAGFS*PSSK | 182 kDa tankyrase 1-binding protein |
| WLDDLLAS*PPPSGGGAR | 182 kDa tankyrase 1-binding protein |
| YESQEPLAGQES*PLPLATR | 182 kDa tankyrase 1-binding protein |
| SGCSEAQPPES*PETR | Transforming acidic coiled-coil-containing protein 3 |
| FIQELSGSS*PK | Transcription factor AP-4 |
| SGYSSPGS*PGTPGSR | Microtubule-associated protein tau |
| SPVVSGDTS*PR | Microtubule-associated protein tau |
| RAVSEGCAS*EDEVEGEA | TBC1 domain family member 2 |
| TSSTCS*NESLSVGGTSVTPR | TBC1 domain family member 4 |
| EPAITSQNS*PEAR | Transcription elongation factor A protein 1 |
| NNDQPQSANANEPQDSTVNLQS*PLK | Transcription factor 8 |
| DSES*PSQK | Treacle protein |
| LDSS*PSVSSTLAAK | Treacle protein |
| LGAGEGGEAS*VSPEK | Treacle protein |
| LGAGEGGEASVS*PEK | Treacle protein |
| S*PAGPAATPAQAQAASTPR | Treacle protein |
| SSSS*ESEDEDVIPATQCLTPGIR | Treacle protein |
| TQPSSGVDSAVGTLPATS*PQSTSVQAK | Treacle protein |
| S*PSSVTGNALWK | Telomeric repeat binding factor 2 interacting protein 1 |
| S*GEGEVSGLMR | Transcription intermediary factor 1-beta |
| AGSS*PAQGAQNEPPR | Transcription factor 20 |
| LNAS*PAAREEATS*PGAK | Transcription factor 20 |
| QLS*GQSTSSDTTYK | Transcription factor 20 |
| SLT*PPPSSTESK | Transcription factor 20 |
| GPPDFS*S*DEEREPTPVLGSGAAAAGR | Thymopoietin, isoform alpha |
| SSTPLPTISSS*AENTR | Thymopoietin, isoform alpha |
| VPEASSEPFDTSS*PQAGR | Triple homeobox 1 protein |
| ILAT*PPQEDAPSVDIANIR | Transketolase |
| DAPTS*PASVASSSSTPSSK | Transducin-like enhancer protein 3 |
| ESSANNSVS*PSESLR | Transducin-like enhancer protein 3 |
| VS*PAHS*PPENGLDK | Transducin-like enhancer protein 3 |
| YDS*DGDKSDDLVVDVSNEDPATPR | Transducin-like enhancer protein 3 |
| YDSDGDKS*DDLVVDVSNEDPATPR | Transducin-like enhancer protein 3 |
| LDEGT*PPEPK | Talin 2 |
| TTQSMQDFPVVDS*EEEAEEEFQK | Tuftelin-interacting protein 11 |
| FTMDLDS*DEDFSDFDEKT*DDEDFVPSDASPPK | DNA topoisomerase II, alpha isozyme |
| GSVPLS*SS*PPATHFPDETEITNPVPK | DNA topoisomerase II, alpha isozyme |
| KPS*TSDDS*DSNFEK | DNA topoisomerase II, alpha isozyme |
| NENTEGS*PQEDGVELEGLK | DNA topoisomerase II, alpha isozyme |
| SVVS*DLEADDVK | DNA topoisomerase II, alpha isozyme |
| TDDEDFVPSDAS*PPK | DNA topoisomerase II, alpha isozyme |
| TQMAEVLPS*PR | DNA topoisomerase II, alpha isozyme |
| VPDEEENEES*DNEK | DNA topoisomerase II, alpha isozyme |
| AS*GSENEGDYNPGR | DNA topoisomerase II, beta isozyme |
| FDS*NEEDSASVFSPSFGLK | DNA topoisomerase II, beta isozyme |
| VVEAVNS*DSDSEFGIPK | DNA topoisomerase II, beta isozyme |
| T*IDDLEDELYAQK | Tropomyosin alpha 3 chain |
| AADSQNS*GEGNTGAAESSFSQEVSR | Nucleoprotein TPR |
| RS*PS*PYYSR | Arginine/serine-rich splicing factor 10 |
| SPS*PYYSR | Arginine/serine-rich splicing factor 10 |
| DLVLPTQALPAS*PALK | Telomeric repeat binding factor 2 |
| TS*PLVSQNNEQGSTLR | Thyroid receptor interacting protein 8 |
| SES*PPAELPSLR | Thyroid receptor interacting protein 12 |
| TT*PLPPPR | Myeloid/lymphoid or mixed-lineage leukemia protein 4 |
| DIDHETVVEEQIIGENS*PPDYSEYMTGK | Transcriptional repressor protein YY1 |
| YYPTAEEVYGPEVETIVQEEDT*QPLTEPIIKPVK | 116 kDa U5 small nuclear ribonucleoprotein component |
| SQS*MDIDGVSCEK | Ubiquitin conjugation factor E4 B |
| NGS*EADIDEGLYSR | Ubiquitin-activating enzyme E1 |
| AGEQQLS*EPEDMEMEAGDTDDPPR | Ubiquitin carboxyl-terminal hydrolase 7 |
| NHSVNEEEQEEQGEGS*EDEWEQVGPR | Ubiquitin carboxyl-terminal hydrolase 10 |
| TCNS*PQNSTDSVSDIVPDSPFPGALGSDTR | Ubiquitin carboxyl-terminal hydrolase 10 |
| NINMDNDLEVLTSS*PTR | Ubiquitin carboxyl-terminal hydrolase 16 |
| AVPPGNDPVS*PAMVR | Ubiquitin carboxyl-terminal hydrolase 19 |
| SVDQGGGGS*PR | Ubiquitin carboxyl-terminal hydrolase 24 |
| T*ISAQDTLAYATALLNEK | Ubiquitin carboxyl-terminal hydrolase 24 |
| VSDQNS*PVLPK | Ubiquitin carboxyl-terminal hydrolase 24 |
| APAGQEEPGT*PPSSPLSAEQLDR | Uracil-DNA glycosylase |
| TDNSVASS*PSSAISTATPSPK | Ubiquitously transcribed X chromosome tetratricopeptide |
| | repeat protein |
| DCDPGS*PR | Vigilin |
| VATLNS*EEESDPPTYK | Vigilin |
| LCDDGPQLPTS*PR | Vinexin |
| SPADPTDLGGQTS*PR | Vinexin |
| SS*SLQGMDMASLPPR | WD-repeat protein WDC146 |
| SPAAPYFLGSSFS*PVR | Wee1-like protein kinase |
| SEAAAPHTDAGGGLS*S*DEEEGTSSQAEAAR | DNA-repair protein complementing XP-C cells |
| ELTPAS*PTCTNSVSK | DNA-repair protein complementing XP-G cells |
| FDSSLLSS*DDETK | DNA-repair protein complementing XP-G cells |
| INSSTENS*DEGLK | DNA-repair protein complementing XP-G cells |
| NAPAAVDEGSIS*PR | DNA-repair protein complementing XP-G cells |
| TEKEPDAT*PPS*PR | DNA-repair protein complementing XP-G cells |
| TLLAMQAALLGS*S*S*EEELESENRR | DNA-repair protein complementing XP-G cells |
| NEMGIPQQTTS*PENAGPQNTK | Hypothetical protein KIAA0008 |
| SEPSGEINIDSS*GETVGSGER | Hypothetical protein KIAA0056 |
| SLGVLPFTLNSGS*PEK | Hypothetical protein KIAA0056 |
| SPAVATSTAAPPPPSS*PLPSK | Hypothetical protein KIAA0144 |
| STSAPQMS*PGSSDNQSSSPQPAQQK | Hypothetical protein KIAA0144 |
| YPSSISSS*PQK | Hypothetical protein KIAA0144 |
| ASDSSS*PSCSSGPR | Hypothetical zinc finger protein KIAA0211 |
| GSPSVAASS*PPAIPK | Hypothetical zinc finger protein KIAA0211 |
| MSDYS*PNSTGSVQNTSR | Putative deoxyribonuclease KIAA0218 |
| ASEGLDACAS*PTK | Hypothetical zinc finger protein KIAA0222 |
| ADSGPTQPPLSLS*PAPETK | Hypothetical protein KIAA0310 |
| QEPGGS*HGSET*EDTGR | Hypothetical protein KIAA0553 |
| QAS*T*DAGTAGALTPQHVR | 65 kDa Yes-associated protein |
| GGLLTSEEDSGFSTS*PK | Zinc finger protein 148 |
| GPLEQNQTIS*PLSTYEESK | Zinc finger protein 148 |
| LSS*FSHK | Zinc finger protein 198 |
| MTGSAPPPS*PTPNK | Zinc finger protein 198 |
| AGAES*PTMSVDGR | Zinc finger protein 217 |
| DVTGS*PPAK | Zinc finger protein 217 |
| QS*PPGPGK | Zinc finger protein 217 |
| TSVS*PAPDK | Zinc finger protein 217 |
| S*ALNVHHK | Zinc finger protein 255 |
| SAPTAPT*PPPPPPPATPR | Zinc finger protein 261 |
| LDEDEDEDDADLSKYNLDAS*EEEDSNK | Zinc finger protein 265 |
| YNLDAS*EEEDSNK | Zinc finger protein 265 |
| EGAS*PVTEVR | Zinc finger protein 295 |
| ESEVCPVPTNSPS*PPPLPPPPPLPK | Zinc finger protein 295 |
| IQPLEPDS*PTGLSENPTPATEK | Zinc finger protein 295 |
| SFS*ASQSTDR | Zinc finger protein 295 |
| SLS*MDSQVPVYSPSIDLK | Zinc finger protein 295 |
| TEPSS*PLSDPSDIIR | Zinc finger protein 295 |
| DGPEPPS*PAK | Zinc finger protein 335 |
| GPASQFYITPSTSLS*PR | Nuclear protein ZAP3 |
| SVGDDEELQQNESGTS*PK | Zinc finger protein 40 |
| ADPGEDDLGGTVDIVES*EPENDHGVELLDQNSSIR | Zinc finger X-chromosomal protein |
| AYS*PEYR | Tight junction protein ZO-2 |
| GSYGS*DAEEEEYR | Tight junction protein ZO-2 |
| SPS*PEPR | Tight junction protein ZO-2 |
| GPPASS*PAPAPKFS*PVTPK | Zyxin |
| S*PGAPGPLTLK | Zyxin |
| S*PILLPK | Cytoskeleton-like bicaudal D protein homolog 2 |
| KTSS*DDES*EEDEDDLLQR | WD-repeat protein CGI-48 |
| NSSS*PVSPASVPGQR | Protein C14orf4 |
| RNS*SS*PVSPASVPGQR | Protein C14orf4 |
| RNS*SSPVS*PASVPGQR | Protein C14orf4 |
| QEAIPDLEDSPPVS*DSEEQQESAR | Death associated transcription factor 1 |
| S*PPEGDTTLFLSR | Death associated transcription factor 1 |
| TAAPS*PSLLYK | Death associated transcription factor 1 |
| SLSNS*NPDISGTPTSPDDEVR | Dedicator of cytokinesis protein 7 |
| SLSNSNPDISGTPTS*PDDEVR | Dedicator of cytokinesis protein 7 |
| LGAS*QER | Transcription elongation factor B polypeptide 3 |
| GS*DGEDSASGGK | Separin |
| SSSLGS*YDDEQEDLTPAQLTR | Protein FAM13A1 |
| SASEHSSS*AES*ER | Formin binding protein 3 |
| ENSGPVENGVS*DQEGEEQAR | Gem-associated protein 5 |
| AQSNGSGNGS*DSEMDTSSLER | Glucocorticoid receptor DNA binding factor 1 |
| TSFSVGS*DDELGPIR | Glucocorticoid receptor DNA binding factor 1 |
| AQS*SPAAPASLSAPEPASQAR | Histone deacetylase 7a |
| AQSS*PAAPASLSAPEPASQAR | Histone deacetylase 7a |
| TQT*PPLGQTPQLGLK | Eukaryotic translation initiation factor 4 gamma 2 |
| ASMSEFLES*EDGEVEQQR | Polycomb protein SUZ12 |
| SSS*PIPLTPSK | Male-specific lethal 3-like 1 |
| DLRS*SS*PR | Mitogen-activated protein kinase kinase kinase kinase 1 |
| AASSLNLS*NGETESVK | Mitogen-activated protein kinase kinase kinase kinase 4 |
| TTS*RS*PVLSR | Mitogen-activated protein kinase kinase kinase kinase 4 |
| EETEYEYS*GS*EEEDDSHGEEGEPSSIMNVPGESTLR | Mitogen-activated protein kinase kinase kinase kinase 6 |
| LDSS*PVLSPGNK | Mitogen-activated protein kinase kinase kinase kinase 6 |
| SPVPSPGSSS*PQLQVK | Molecule interacting with Rab13 |
| VEQMPQAS*PGLAPR | Molecule interacting with Rab13 |
| VPAMPGS*PVEVK | Protein CBFA2T2 |
| FS*PDSQYIDNR | Partitioning-defective 3 homolog |
| GLIVYCVTS*PK | PDZ domain containing guanine nucleotide exchange |
| | factor 2 |
| MAPPVDDLS*PK | PHD finger protein 3 |
| QLQEDQENNLQDNQTSNSS*PCR | PHD finger protein 3 |
| NSADDEELTNDS*LTLSQSK | PHD finger protein 14 |
| GVQVPAS*PDTVPQPSLR | PHD finger protein 16 |
| ETVQTTQS*PTPVEK | Putative RNA-binding protein 16 |
| NSLLAGGDDDTMSVIS*GISSR | Cohesin subunit SA-2 |
| NSLLAGGDDDTMSVISGISS*R | Cohesin subunit SA-2 |
| LFQLGPPS*PVK | Securin |
| AAEKPEEEESAAEEESNS*DEDEVIPDIDVEVDVDELNQEQVADLNK | Splicing factor, arginine/serine-rich 16 |
| ITFITSFGGS*DEEAAAAAAAAAASGVTTGKPPAPPQPGGPAPGR | Splicing factor, arginine/serine-rich 16 |
| SQS*PSPS*PAREK | Splicing factor, arginine/serine-rich 16 |
| SQSPS*PSPAR | Splicing factor, arginine/serine-rich 16 |
| SRS*PT*PGR | Splicing factor, arginine/serine-rich 16 |
| GTMDDISQEEGSS*QGEDSVSGSQR | Structural maintenance of chromosome 1-like 1 protein |
| GTMDDISQEEGSSQGEDS*VSGSQR | Structural maintenance of chromosome 1-like 1 protein |
| MEEESQS*QGR | Structural maintenance of chromosome 1-like 1 protein |
| GDVEGSQSQDEGEGS*GESER | Structural maintenance of chromosome 3 |
| GSGS*QSSVPSVDQFTGVGIR | Structural maintenance of chromosome 3 |
| KGDVEGS*QS*QDEGEGSGESER | Structural maintenance of chromosome 3 |
| EEGPPPPS*PDGASSDAEPEPPSGR | Structural maintenance of chromosomes 4-like 1 protein |
| REEGPPPPS*PDGASS*DAEPEPPSGR | Structural maintenance of chromosomes 4-like 1 protein |
| TES*PATAAETASEELDNR | Structural maintenance of chromosomes 4-like 1 protein |
| ANT*PDS*DITEKTEDSSVPETPDNER | SWI/SNF-related, actin-dependent regulator of chromatin |
| | subfamily A containing DEAD/H box 1 |
| IEEAPEATPQPSQPGPSS*PISLSAEEENAEGEVSR | SWI/SNF-related, actin-dependent regulator of chromatin |
| | subfamily A containing DEAD/H box 1 |
| NKIEEAPEATPQPSQPGPSS*PIS*LS*AEEENAEGEVSR | SWI/SNF-related, actin-dependent regulator of chromatin |
| | subfamily A containing DEAD/H box 1 |
| TEDSS*VPETPDNER | SWI/SNF-related, actin-dependent regulator of chromatin |
| | subfamily A containing DEAD/H box 1 |
| T*PPVVIK | Synapse associated protein 1 |
| KAEDS*DS*EPEPEDNVR | 5′-3′ exoribonuclease 2 |
| NS*PGSQVASNPR | 5′-3′ exoribonuclease 2 |
| EES*DEEEEDDEESGR | GPN: BC0119231 |
| GDSIEEILADS*EDEEDNEEEER | GPN: BC012745_1 |
| EPTPSIASDIS*LPIATQELR | GPN: BC013957_1 |
| SSFYSGGWQEGSSS*PR | GPN: BC015239_1 |
| YNAVLGFGALTPTS*PQSSHPDS*PENEK | GPN: BC015714_1 |
| LLSS*ESEDEEEFIPLAQR | GPN: BC016470_1 |
| MAGNEALS*PTSPFR | GPN: BC017269_1 |
| DSDSGSDSDS*DQENAASGSNASGSESDQDERGDSGQPSNK | GPN: BC018147_1 |
| GS*DSEDEVLR | GPN: BC018147_1 |
| GSDS*EDEVLR | GPN: BC018147_1 |
| KNAIAS*DSEADS*DTEVPK | GPN: BC018147_1 |
| LTS*DEEGEPSGK | GPN: BC018147_1 |
| NAIAS*DSEADSDTEVPK | GPN: BC018147_1 |
| NAIASDSEADS*DTEVPK | GPN: BC018147_1 |
| LEDSEVRS*VAS*NQSEMEFSSLQDMPK | GPN: BC018269_1 |
| S*VASNQSEMEFSSLQDMPK | GPN: BC018269_1 |
| SVAS*NQSEMEFSSLQDMPK | GPN: BC018269_1 |
| YLPLNTALYEPPLDPELPALDS*DGDS*DDGEDGRGDEK | GPN: BC020954_1 |
| S*FEVEEVETPNSTPPR | GPN: BC021192_1 |
| FLNILLLIPTLQS*EGHIR | GPN: BC021969_1 |
| ISNLS*PEEEQGLWK | GPN: BC026013_1 |
| DMDEPS*PVPNVEEVTLPK | GPN: BC026222_1 |
| S*PSPSPTPEAK | GPN: BC026222_1 |
| SPS*PSPTPEAK | GPN: BC026222_1 |
| TLTDEVNS*PDSDR | GPN: BC026222_1 |
| VNQSALEAVTPS*PSFQQR | GPN: BC028599_1 |
| ASVLSQS*PR | GPN: BC031107_1 |
| QMS*VPGIFNPHEIPEEMCD | GPN: BC032847_1 |
| AEQGS*EEEGEGEEEEEEGGESK | GPN: BC034488_1 |
| KSS*VTEE | GPN: BC036379_1 |
| EALGLGPPAAQLT*PPPAPVGLR | GPN: BC037428_1 |
| AGVNSDS*PNNCSGK | GPN: BC038297_1 |
| SS*ENNGTLVSK | GPN: BC038297_1 |
| LTAS*PSDPK | GPN: BC042999_1 |
| LYGS*PTQIGPSYR | GPN: BC042999_1 |
| EGSCIFPEELS*PK | GPN: BC044254_1 |
| ASS*PPDR | GPN: BC050434_1 |
| SSDEENGPPSS*PDLDR | GPN: BC051844_1 |
| SQS*LPTTLLSPVR | GPN: BC052581_1 |
| APS*PPS*RR | GPN: BC052950_1 |
| SPS*GAGEGASCSDGPR | GPN: BC052950_1 |
| SPS*PAPAPAPAAAAGPPTR | GPN: BC053992_1 |
| TSPGTSSAYTSDS*PGSYHNEEDEEEDGGEEGMDEQYR | GPN: BC055396_1 |
| EESS*EDENEVSNILR | GPN: BC057242_1 |
| TAADVVS*PGANSVDSR | GPN: BC057242_1 |
| S*DLLANQSQEVLEER | GPN: BC058039_1 |
| S*GTPTQDEMMDKPTSSSVDTMSLLSK | GPN: BX641025_1 |
| LVT*STTAPNPVR | PIR1: A49724 |
| LVS*PDLQLDAS*VR | PIR1: I38344 |
| NVSES*PNR | PIR1: JC5314 |
| S*ET*PPHWR | PIR1: JC5314 |
| SASS*ES*EAENLEAQPQSTVRPEEIPPIPENR | PIR1: JC5314 |
| ATSS*TQSLAR | PIR2: A42184 |
| TQPDGTSVPGEPAS*PISQR | PIR2: A42184 |
| QQAAYYAQTS*PQGMPQHPPAPQGQ | PIR2: A53184 |
| SCMLTGT*PESVQSAK | PIR2: A53184 |
| TGEDEDEEDNDALLKENES*PDVR | PIR2: A53545 |
| VTNDIS*PESSPGVGR | PIR2: A54103 |
| ESVSTEDLSPPS*PPLPK | PIR2: A56138 |
| RISAS*LSCDSPK | PIR2: A61382 |
| GEDS*AEETEAKPAVVAPAPVVEAVSTPSAAFPSDATAENVK | PIR2: B54857 |
| DLLSDLQDIS*DSER | PIR2: E54024 |
| VPAS*PLPGLER | PIR2: G01025 |
| S*DLPGSDK | PIR2: G01158 |
| TQQSPISNGS*PELGIK | PIR2: G02318 |

TABLE 5A
N-Terminal Peptides - Saccharomyces cerevisiae
N-Terminal a-Amino Group Unblocked

| Protein | Peptide |
|---|---|
| GP: Z75238_1 | MDYERTVLKKRSR |
| PIR1: S69731 | VVVGKSEVR |
| PIR2: S48569 | VFGFTKR |
| PIR2: S50385 | PALLKR |
| PIR2: S52504 | PITIKSR |
| PIR2: S52698 | VAISEVKENPGVNSSNSGAVTR |
| PIR2: S57377 | MQLVPLELNR |
| PIR2: S59436 | PDNNTEQLQGSPSSDQR |
| PIR2: S59832 | GIQEKTLGIR |
| PIR2: S61156 | VQAIKLNDLKNR |
| PIR2: S61160 | AGENPKKEGVDAR |
| PIR2: S61668 | VVNTIYIAR |
| PIR2: S64842 | VNKVVDEVQR |
| PIR2: S65155 | MLVKTISR |
| PIR2: S65218 | MKGTGGVVVGTQNPVR |
| PIR2: S66925 | AKRPLGLGKQSR |
| PIR2: S66937 | TNKSSLKNNR |
| PIR2: S67033 | VAPTALKKATVTPVSGQDGGSSR |
| PIR2: S67052 | VPAESNAVQAKLAKTLQR |
| PIR2: S67059 | VVQKKLR |
| PIR2: S67185 | TKEVPYYCDNDDNNIIR |
| PIR2: S67655 | VGGALICKYLPR |
| PIR2: S67696 | AGSQLKNLKAALKAR |
| PIR2: S67704 | PELTEFQKKR |
| PIR2: S67772 | GSEEDKKLTKKQLKAQQFR |
| PIR2: S78735 | MIEVVVNDR |
| SW: ACH1_YEAST | TISNLLKQR |
| SW: AGM1_YEAST | MKVDYEQLCKLYDDTCR |
| SW: AKR1_YEAST | VNELENVPR |
| SW: ALF_YEAST | GVEQILKR |
| SW: APG8_YEAST | MKSTFKSEYPFEKR |
| SW: ARO8_YEAST | TLPESKDFSYLFSDETNAR |
| SW: ASN1_YEAST | CGIFAAFR |
| SW: ATC6_YEAST | TKKSFVSSPIVR |
| SW: C1TC_YEAST | AGQVLDGKACAQQFR |
| SW: CAJ1_YEAST | VKETEYYDILGIKPEATPTEIKKAYR |
| SW: CAP_YEAST | PDSKYTMQGYNLVKLLKR |
| SW: CB34_YEAST | VTSNVVLVSGEGER |
| SW: CBS_YEAST | TKSEQQADSR |
| SW: CHD1_YEAST | AAKDISTEVLQNPELYGLR |
| SW: COPA_YEAST | MKMLTKFESKSTR |
| SW: COPP_YEAST | MKLDIKKTFSNR |
| SW: CYC1_YEAST | TEFKAGSAKKGATLFKTR |
| SW: CYC7_YEAST | AKESTGFKPGSAKKGATLFKTR |
| SW: CYP6_YEAST | TRPKTFFDISIGGKPQGR |
| SW: DBP3_YEAST | TKEEIADKKR |
| SW: DCUP_YEAST | GNFPAPKNDLILR |
| SW: DHAS_YEAST | AGKKIAGVLGATGSVGQR |
| SW: DHE2_YEAST | MLFDNKNR |
| SW: E2BE_YEAST | AGKKGQKKSGLGNHGKNSDMDVEDR |
| SW: EF2_YEAST | VAFTVDQMR |
| SW: EGD1_YEAST | PIDQEKLAKLQKLSANNKVGGTR |
| SW: ELO1_YEAST | VSDWKNFCLEKASR |
| SW: ENO1_YEAST | AVSKVYAR |
| SW: ERV2_YEAST | MKQIVKR |
| SW: FHP_YEAST | MLAEKTR |
| SW: GLO2_YEAST | MQVKSIKMR |
| SW: GLO3_YEAST | SNDEGETFATEQTTQQVFQKLGSNMENR |
| SW: GLY1_YEAST | TEFELPPKYITAANDLR |
| SW: HIS7_YEAST | TEQKALVKR |
| SW: HIS8_YEAST | VFDLKR |
| SW: HMD1_YEAST | PPLFKGLKQMAKPIAYVSR |
| SW: HOSC_YEAST | TAAKPNPYAAKPGDYLSNVNNFQLIDSTLR |
| SW: IF1A_YEAST | GKKNTKGGKKGR |
| SW: ILV3_YEAST | GLLTKVATSR |
| SW: KEL3_YEAST | AKKNKKDKEAKKAR |
| SW: KIN2_YEAST | PNPNTADYLVNPNFR |
| SW: KRE2_YEAST | ALFLSKR |
| SW: LA17_YEAST | GLLNSSDKEIIKR |
| SW: LAG1_YEAST | TSATDKSIDR |
| SW: LEO1_YEAST | SSESPQDQPQKEQISNNVGVTTNSTSNEETSR |
| SW: METE_YEAST | VQSAVLGFPR |
| SW: MFT1_YEAST | PLSQKQIDQVR |
| SW: MPG1_YEAST | MKGLILVGGYGTR |
| SW: MYS3_YEAST | AVIKKGAR |
| SW: NCE2_YEAST | MLALADNILR |
| SW: NHPB_YEAST | AATKEAKQPKEPKKR |
| SW: NOG1_YEAST | MQLSWKDIPTVAPANDLLDIVLNR |
| SW: OM22_YEAST | VELTEIKDDVVQLDEPQFSR |
| SW: OM70_YEAST | MKSFITR |
| SW: ORM1_YEAST | TELDYQGTAEAASTSYSR |
| SW: PCNA_YEAST | MLEAKFEEASLFKR |
| SW: PDR3_YEAST | MKVKKSTR |
| SW: PH81_YEAST | MKFGKYLEAR |
| SW: PH88_YEAST | MNPQVSNIIIMLVMMQLSR |
| SW: PMG1_YEAST | PKLVLVR |
| SW: POR1_YEAST | SPPVYSDISR |
| SW: PUF6_YEAST | APLTKKTNGKR |
| SW: PUR2_YEAST | MLNILVLGNGAR |
| SW: PUR8_YEAST | PDYDNYTTPLSSR |
| SW: PWP1_YEAST | MISATNWVPR |
| SW: PWP2_YEAST | MKSDFKFSNLLGTVYR |
| SW: R142_YEAST | ANDLVQAR |
| SW: R15A_YEAST | GAYKYLEELQR |
| SW: R15B_YEAST | GAYKYLEELER |
| SW: R24A_YEAST | MKVEIDSFSGAKIYPGR |
| SW: R24B_YEAST | MKVEVDSFSGAKIYPGR |
| SW: R261_YEAST | AKQSLDVSSDR |
| SW: R37A_YEAST | GKGTPSFGKR |
| SW: RAS2_YEAST | PLNKSNIR |
| SW: RIB4_YEAST | AVKGLGKPDQVYDGSKIR |
| SW: RL25_YEAST | APSAKATAAKKAVVKGTNGKKALKVR |
| SW: RL27_YEAST | AKFLKAGKVAVVVR |
| SW: RL31_YEAST | AGLKDVVTR |
| SW: RL35_YEAST | AGVKAYELR |
| SW: RL39_YEAST | AAQKSFR |
| SW: RL44_YEAST | VNVPKTR |
| SW: RL5_YEAST | AFQKDAKSSAYSSR |
| SW: RL6A_YEAST | SAQKAPKWYPSEDVAALKKTR |
| SW: RL6B_YEAST | TAQQAPKWYPSEDVAAPKKTR |
| SW: RL7A_YEAST | AAEKILTPESQLKKSKAQQKTAEQVAAER |
| SW: RL7B_YEAST | STEKILTPESQLKKTKAQQKTAEQIAAER |
| SW: RL8A_YEAST | APGKKVAPAPFGAKSTKSNKTR |
| SW: RL9A_YEAST | MKYIQTEQQIEVPEGVTVSIKSR |
| SW: RNT1_YEAST | GSKVAGKKKTQNDNKLDNENGSQQR |
| SW: RPB1_YEAST | VGQQYSSAPLR |
| SW: RPC1_YEAST | MKEVVVSETPKR |
| SW: RPD3_YEAST | VYEATPFDPITVKPSDKR |
| SW: RPF1_YEAST | ALGNEINITNKLKR |
| SW: RPN7_YEAST | VDVEEKSQEVEYVDPTVNR |
| SW: RS1B_YEAST | MLMPKQER |
| SW: RS3_YEAST | VALISKKR |
| SW: RS3A_YEAST | AVGKNKR |
| SW: SDS3_YEAST | AIQKVSNKDLSR |
| SW: SIS1_YEAST | VKETKLYDLLGVSPSANEQELKKGYR |
| SW: SLA1_YEAST | TVFLGIYR |
| SW: SMD1_YEAST | MKLVNFLKKLR |
| SW: SOF1_YEAST | MKIKTIKR |
| SW: SOK2_YEAST | PIGNPINTNDIKSNR |
| SW: SPB1_YEAST | GKTQKKNSKGR |
| SW: SPC3_YEAST | MFSFVQR |
| SW: SR54_YEAST | VLADLGKR |
| SW: SR68_YEAST | VAYSPIIATYGNR |
| SW: SRB2_YEAST | GKSAVIFVER |
| SW: ST12_YEAST | MKVQITNSR |
| SW: STL1_YEAST | MKDLKLSNFKGKFISR |
| SW: SWI6_YEAST | ALEEVVR |
| SW: SYAC_YEAST | TIGDKQKWTATNVR |
| SW: SYSC_YEAST | MLDINQFIEDKGGNPELIR |
| SW: T2FC_YEAST | VATVKR |
| SW: TCPG_YEAST | MQAPVVFMNASQER |
| SW: THRC_YEAST | PNASQVYR |
| SW: TKT1_YEAST | TQFTDIDKLAVSTIR |
| SW: TRF4_YEAST | GAKSVTASSSKKIKNR |
| SW: TRM8_YEAST | MKAKPLSQDPGSKR |
| SW: TTP1_YEAST | MLLTKR |
| SW: TYSY_YEAST | TMDGKNKEEEQYLDLCKR |
| SW: UFD2_YEAST | TAIEDILQITTDPSDTR |
| SW: UGA2_YEAST | TLSKYSKPTLNDPNLFR |
| SW: VAN1_YEAST | GMFFNLR |
| SW: VATB_YEAST | VLSDKELFAINKKAVEQGFNVKPR |
| SW: VP35_YEAST | AYADSPENAIAVIKQR |
| SW: YAD1_YEAST | VDVQKR |
| SW: YB01_YEAST | AFLNIFKQKR |
| SW: YB09_YEAST | TFMQQLQEAGER |
| SW: YBV2_YEAST | VEFSLKKAR |
| SW: YBY7_YEAST | VVLDKKLLER |
| SW: YCY4_YEAST | VSLFKR |
| SW: YEJ4_YEAST | MNGLVLGATGLCGGGFLR |
| SW: YEM6_YEAST | PPVSASKAKR |
| SW: YEV6_YEAST | PQNDYIER |
| SW: YFA7_YEAST | TANNDDDIKSPIPITNKTLSQLKR |
| SW: YG1I_YEAST | AKTIKVIR |
| SW: YG38_YEAST | PSLSQPFR |
| SW: YG3A_YEAST | MLFNINR |
| SW: YG3C_YEAST | TKKKAATNYAER |
| SW: YG3J_YEAST | VLKSTSANDVSVYQVSGTNVSR |
| SW: YGC9_YEAST | VNETGESQKAAKGTPVSGKVWKAEKTPLR |
| SW: YGF0_YEAST | AAQNAFEQKKR |
| SW: YGK1_YEAST | TAVNIWKPEDNIPR |
| SW: YGZ6_YEAST | GVSANLFVKQR |
| SW: YHD0_YEAST | SISSDEAKEKQLVEKAELR |
| SW: YIK8_YEAST | VGSKDIDLFNLR |
| SW: YIN0_YEAST | PEQAQQGEQSVKR |
| SW: YIV6_YEAST | GKVILITGASR |
| SW: YJ58_YEAST | MLKDLVR |
| SW: YJG8_YEAST | MKVVKEFSVCGGR |
| SW: YKV5_YEAST | MQKGNIR |
| SW: YL22_YEAST | PINQPSGQIKLTNVSLVR |
| SW: YMJ3_YEAST | AKKKSKSR |
| SW: YMY0_YEAST | SPMKVAVVGASGKVGR |
| SW: YN63_YEAST | VNFDLGQVGEVFR |
| SW: YN8U_YEAST | GTGKKEKSR |
| SW: YNK8_YEAST | AIENIYIAR |
| SW: YNM3_YEAST | TISLSNIKKR |
| SW: YNN2_YEAST | AKKAIDSR |
| SW: YNQ6_YEAST | GLDQDKIKKR |
| SW: YP46_YEAST | APTNLTKKPSQYKQSSR |
| SW: ZRC1_YEAST | MITGKELR |

TABLE 5B
N-Terminal Peptides - Saccharomyces cerevisiae
N-Terminal a-Amino Group Acetylated

| Protein | Peptide |
|---|---|
| GP: AB017593_1 | SDWDTNTIIGSR |
| GP: L01880_1 | SQGTLYLNR |
| PIR1: R3BY33 | MDNKTPVTLAKVIKVLGR |
| PIR1: R5BY16 | STKAQNPMR |
| PIR1: S53543 | MFKKFTR |
| PIR2: S51406 | SQLPTDFASLIKR |
| PIR2: S54047 | SNLYKIGTETR |
| PIR2: S57985 | SELEATIR |
| PIR2: S61039 | ATFNPQNEMENQAR |
| PIR2: S61625 | MDQSVEDLFGALR |
| PIR2: S65214 | TSLYAPGAEDIR |
| PIR2: S65214 | TSLYAPGAEDIR |
| PIR2: S67177 | SELLAIPLKR |
| PIR2: S67177 | SELLAIPLKR |
| PIR2: S70126 | SESVKENVTPTR |
| SW: ACT_YEAST | MDSEVAALVIDNGSGMCKAGFAGDDAPR |
| SW: AIP1_YEAST | SSISLKEIIPPQPSTQR |
| SW: ALG3_YEAST | MEGEQSPQGEKSLQR |
| SW: AR20_YEAST | SQSLRPYLTAVR |
| SW: ARE2_YEAST | MDKKKDLLENEQFLR |
| SW: AROG_YEAST | SESPMFAANGMPKVNQGAEEDVR |
| SW: ATC1_YEAST | SDNPFNASLLDEDSNR |
| SW: ATP7_YEAST | SLAKSAANKLDWAKVISSLR |
| SW: BAS1_YEAST | SNISTKDIR |
| SW: BEM1_YEAST | MLKNFKLSKR |
| SW: CAPB_YEAST | SDAQFDAALDLLR |
| SW: CC11_YEAST | SGIIDASSALR |
| SW: CC12_YEAST | SAATATAAPVPPPVGISNLPNQR |
| SW: CC28_YEAST | SGELANYKR |
| SW: CDC3_YEAST | SLKEEQVSIKQDPEQEER |
| SW: CET1_YEAST | SYTDNPPQTKR |
| SW: CH10_YEAST | STLLKSAKSIVPLMDR |
| SW: CHMU_YEAST | MDFTKPETVLNLQNIR |
| SW: CISY_YEAST | SAILSTTSKSFLSR |
| SW: CK12_YEAST | SQVQSPLTATNSGLAVNNNTMNSQMPNR |
| SW: CLC1_YEAST | SEKFPPLEDQNIDFTPNDKKDDDTDFLKR |
| SW: COAC_YEAST | SEESLFESSPQKMEYEITNYSER |
| SW: CYAA_YEAST | SSKPDTGSEISGPQR |
| SW: CYPH_YEAST | SQVYFDVEADGQPIGR |
| SW: DCP1_YEAST | SEITLGKYLFER |
| SW: DEC1_YEAST | SDKIQEEILGLVSR |
| SW: DHH1_YEAST | GSINNNFNTNNNSNTDLDR |
| SW: DPD2_YEAST | MDALLTKFNEDR |
| SW: DPOA_YEAST | SSKSEKLEKLR |
| SW: E2BA_YEAST | SEFNITETYLR |
| SW: EF1G_YEAST | SQGTLYANFR |
| SW: EF1H_YEAST | SQGTLYINR |
| SW: EGD2_YEAST | SAIPENANVTVLNKNEKKAR |
| SW: ERF2_YEAST | SDSNQGNNQQNYQQYSQNGNQQQGNNR |
| SW: FAS1_YEAST | MDAYSTR |
| SW: FKBP_YEAST | SEVIEGNVKIDR |
| SW: FOLD_YEAST | AIELGLSR |
| SW: FPPS_YEAST | ASEKEIR |
| SW: GALY_YEAST | SAAPVQDKDTLSNAER |
| SW: GBLP_YEAST | ASNEVLVLR |
| SW: GC20_YEAST | ASIGSQVR |
| SW: GCN1_YEAST | TAILNWEDISPVLEKGTR |
| SW: GCS1_YEAST | SDWKVDPDTR |
| SW: GLNA_YEAST | AEASIEKTQILQKYLELDQR |
| SW: GLO3_YEAST | SNDEGETFATEQTTQQVFQKLGSNMENR |
| SW: GLY1_YEAST | TEFELPPKYITAANDLR |
| SW: GNA1_YEAST | SLPDGFYIR |
| SW: GSHR_YEAST | MLSATKQTFR |
| SW: GSP1_YEAST | SAPAANGEVPTFKLVLVGDGGTGKTTFVKR |
| SW: GUP1_YEAST | SLISILSPLITSEGLDSR |
| SW: H2A1_YEAST | SGGKGGKAGSAAKASQSR |
| SW: H2B2_YEAST | SSAAEKKPASKAPAEKKPAAKKTSTSVDGKKR |
| SW: HS77_YEAST | MLAAKNILNR |
| SW: HS78_YEAST | STPFGLDLGNNNSVLAVAR |
| SW: HXT2_YEAST | SEFATSR |
| SW: IF34_YEAST | SEVAPEEIIENADGSR |
| SW: IM09_YEAST | MDALNSKEQQEFQKVVEQKQMKDFMR |
| SW: IMA1_YEAST | MDNGTDSSTSKFVPEYR |
| SW: IMB1_YEAST | STAEFAQLLENSILSPDQNIR |
| SW: KM8S_YEAST | TTASSSASQLQQR |
| SW: LAG1_YEAST | TSATDKSIDR |
| SW: LAH1_YEAST | SEKPQQEEQEKPQSR |
| SW: LSM3_YEAST | METPLDLLKLNLDER |
| SW: LTV1_YEAST | SKKFSSKNSQR |
| SW: MAD2_YEAST | SQSISLKGSTR |
| SW: MP10_YEAST | SELFGVLKSNAGR |
| SW: MS16_YEAST | MLTSILIKGR |
| SW: MYS2_YEAST | SFEVGTR |
| SW: N157_YEAST | MYSTPLKKR |
| SW: NHPX_YEAST | SAPNPKAFPLADAALTQQILDVVQQAANLR |
| SW: NOP8_YEAST | MDSVIQKR |
| SW: NTF2_YEAST | SLDFNTLAQNFTQFYYNQFDTDR |
| SW: NU84_YEAST | MELSPTYQTER |
| SW: NUT1_YEAST | MEKESVYNLALKCAER |
| SW: OM06_YEAST | MDGMFAMPGAAAGAASPQQPKSR |
| SW: PAT1_YEAST | SFFGLENSGNAR |
| SW: PEXE_YEAST | SDVVSKDR |
| SW: PFD1_YEAST | SQIAQEMTVSLR |
| SW: PFD3_YEAST | MDTLFNSTEKNAR |
| SW: PGK_YEAST | SLSSKLSVQDLDLKDKR |
| SW: PGM1_YEAST | SLLIDSVPTVAYKDQKPGTSGLR |
| SW: PMT1_YEAST | SEEKTYKR |
| SW: PNPH_YEAST | SDILNVSQQR |
| SW: PP12_YEAST | MDSQPVDVDNIIDR |
| SW: PROA_YEAST | SSSQQIAKNAR |
| SW: PROF_YEAST | SWQAYTDNLIGTGKVDKAVIYSR |
| SW: PRP2_YEAST | SSITSETGKR |
| SW: PRP5_YEAST | METIDSKQNINR |
| SW: PSA3_YEAST | TSIGTGYDLSNSVFSPDGR |
| SW: PSA6_YEAST | SGAAAASAAGYDR |
| SW: PSB2_YEAST | MDIILGIR |
| SW: PUR4_YEAST | TDYILPGPKALSQFR |
| SW: PUR7_YEAST | SITKTELDGILPLVAR |
| SW: PUS1_YEAST | SEENLRPAYDDQVNEDVYKR |
| SW: PYR1_YEAST | ATIAPTAPITPPMESTGDR |
| SW: PYRF_YEAST | SKATYKER |
| SW: R10A_YEAST | SKITSSQVR |
| SW: R141_YEAST | SNVVQAR |
| SW: R142_YEAST | ANDLVQAR |
| SW: R14A_YEAST | STDSIVKASNWR |
| SW: R161_YEAST | SWEGFKKAINR |
| SW: R167_YEAST | SFKGFTKAVSR |
| SW: RCL1_YEAST | SSSAPKYTTFQGSQNFR |
| SW: REP2_YEAST | MDDIETAKNLTVKAR |
| SW: RFC2_YEAST | MFEGFGPNKKR |
| SW: RHO1_YEAST | SQQVGNSIR |
| SW: RHO3_YEAST | SFLCGSASTSNKPIER |
| SW: RIR1_YEAST | MYVYKR |
| SW: RIR4_YEAST | MEAHNQFLKTFQKER |
| SW: RL11_YEAST | SAKAQNPMR |
| SW: RL23_YEAST | SGNGAQGTKFR |
| SW: RL6A_YEAST | SAQKAPKVVYPSEDVAALKKTR |
| SW: RL73_YEAST | SSTQDSKAQTLNSNPEILLR |
| SW: RL7A_YEAST | AAEKILTPESQLKKSKAQQKTAEQVAAER |
| SW: RL7B_YEAST | STEKILTPESQLKKTKAQQKTAEQIAAER |
| SW: RPA2_YEAST | SKVIKPPGQAR |
| SW: RPB3_YEAST | SEEGPQVKIR |
| SW: RPB8_YEAST | SNTLFDDIFQVSEVDPGR |
| SW: RPC5_YEAST | SNIVGIEYNR |
| SW: RPN2_YEAST | SLTTAAPLLALLR |
| SW: RPN6_YEAST | SLPGSKLEEAR |
| SW: RR44_YEAST | SVPAIAPR |
| SW: RRP1_YEAST | METSNFVKQLSSNNR |
| SW: RRP4_YEAST | SEVITITKR |
| SW: RRP6_YEAST | TSENPDVLLSR |
| SW: RS11_YEAST | STELTVQSER |
| SW: RS15_YEAST | SQAVNAKKR |
| SW: RS2_YEAST | SAPEAQQQKR |
| SW: RS20_YEAST | SDFQKEKVEEQEQQQQQIIKIR |
| SW: RS21_YEAST | MENDKGQLVELYVPR |
| SW: RS24_YEAST | SDAVTIR |
| SW: RS28_YEAST | MDSKTPVTLAKVIKVLGR |
| SW: SAHH_YEAST | SAPAQNYKIADISLAAFGR |
| SW: SC17_YEAST | SDPVELLKR |
| SW: SC23_YEAST | MDFETNEDINGVR |
| SW: SE33_YEAST | SYSAADNLQDSFQR |
| SW: SEC1_YEAST | SDLIELQR |
| SW: SEC2_YEAST | MDASEEAKR |
| SW: SEC8_YEAST | MDYLKPAQKGR |
| SW: SFT2_YEAST | SEEPPSDQVNSLR |
| SW: SMI1_YEAST | MDLFKR |
| SW: SNC2_YEAST | SSSVPYDPYVPPEESNSGANPNSQNKTAALR |
| SW: SPK1_YEAST | MENITQPTQQSTQATQR |
| SW: SPT6_YEAST | MEETGDSKLVPR |
| SW: SR21_YEAST | SVKPIDNYITNSVR |
| SW: SSB1_YEAST | SAEIEEATNAVNNLSINDSEQQPR |
| SW: STDH_YEAST | SIVYNKTPLLR |
| SW: SUM1_YEAST | SENTTAPSDNITNEQR |
| SW: SYG_YEAST | SVEDIKKAR |
| SW: SYLC_YEAST | SSGLVLENTAR |
| SW: TBF1_YEAST | MDSQVPNNNESLNR |
| SW: TCPA_YEAST | SQLFNNSR |
| SW: TCPB_YEAST | SVQIFGDQVTEER |
| SW: TCPD_YEAST | SAKVPSNATFKNKEKPQEVR |
| SW: TCPZ_YEAST | SLQLLNPKAESLR |
| SW: TFC5_YEAST | SSIVNKSGTR |
| SW: THI7_YEAST | SFGSKVSR |
| SW: THIL_YEAST | SQNVYIVSTAR |
| SW: TKT1_YEAST | TQFTDIDKLAVSTIR |
| SW: TPS2_YEAST | TTTAQDNSPKKR |
| SW: TREA_YEAST | SQVNTSQGPVAQGR |
| SW: UBA1_YEAST | SSNNSGLSAAGEIDESLYSR |
| SW: UBP6_YEAST | SGETFEFNIR |
| SW: VATA_YEAST | AGAIENAR |
| SW: VATE_YEAST | SSAITALTPNQVNDELNKMQAFIR |
| SW: VTC1_YEAST | SSAPLLQR |
| SW: YAD6_YEAST | STTVEKIKAIEDEMAR |
| SW: YBD6_YEAST | STGITYDEDR |
| SW: YBM6_YEAST | SANDYYGGTAGEKSQYSR |
| SW: YBN2_YEAST | SNITYVKGNILKPKSYAR |
| SW: YBV1_YEAST | MEKLLQWSIANSQGDKEAMAR |
| SW: YFL8_YEAST | SYKANQPSPGEMPKR |
| SW: YG1G_YEAST | ANSKFGYVR |
| SW: YG5U_YEAST | STATIQDEDIKFQR |
| SW: YGK1_YEAST | TAVNIWKPEDNIPR |
| SW: YHD1_YEAST | SSQPSFVTIR |
| SW: YHP9_YEAST | SLTEQIEQFASR |
| SW: YIE4_YEAST | STSVPVKKALSALLR |
| SW: YIK3_YEAST | SGSTESKKQPR |
| SW: YJA7_YEAST | CSRGGSNSR |
| SW: YJF4_YEAST | SSESGKPIAKPIR |
| SW: YJK9_YEAST | SSLSDQLAQVASNNATVALDR |
| SW: YK10_YEAST | SYLPTYSNDLPAGPQGQR |
| SW: YKA8_YEAST | STIKPSPSNNNLKVR |
| SW: YKL7_YEAST | SDKVINPQVAWAQR |
| SW: YL09_YEAST | SIDLKKR |
| SW: YL86_YEAST | MEKSIAKGLSDKLYEKR |
| SW: YM11_YEAST | MDAGLSTMATR |
| SW: YM28_YEAST | ADLQKQENSSR |
| SW: YM8W_YEAST | SQPTPIITTKSAAKPKPKIFNLFR |
| SW: YME8_YEAST | MEIYIR |
| SW: YML7_YEAST | SNSNSKKPVANYAYR |
| SW: YMS1_YEAST | SLISAVEDR |
| SW: YNJ9_YEAST | TSKVGEYEDVPEDESR |
| SW: YNU8_YEAST | SANEFYSSGQQGQYNQQNNQER |
| SW: YNZ8_YEAST | MESLFPNKGEIIR |
| SW: YP18_YEAST | SLEAIVFDR |
| SW: YRA1_YEAST | SANLDKSLDEIIGSNKAGSNR |

1. A method for characterizing phosphorylated polypeptides in a sample comprising:
providing a biological sample comprising plurality of polypeptides;
digesting the polypeptides with a protease, thereby generating a plurality of test peptides;
collecting a fraction of test peptides which are enriched for positively charged peptides; and
determining an identifying characteristic of a positively charged peptide in the fraction.

2. The method according to claim 1, wherein collecting the fraction comprises exposing the plurality of test peptides to a strong cation exchanger.

3. The method according to claim 2, further comprising eluting peptides from the strong cation exchanger at pH 3 and collecting eluted peptides which are enriched for phosphorylated peptides.

4. The method according to claim 3, wherein the phosphorylated peptides comprise greater than about 50% of peptides in the initial fraction.

5. The method of claim 1, wherein the identifying characteristic is mass-to-charge ratio.

6. The method of claim 1, wherein the identifying characteristic is a peptide fragmentation pattern.

7. The method of claim 1 wherein the identifying characteristic is the amino acid sequence of the peptide.

8. The method of claim 1, further comprising sequencing substantially all of the positively charged peptides in the enriched subset.

9. The method of claim 1, further comprising determining the mass of substantially all of the positively charged peptides in the enriched subset.

10. The method of claim 1, further comprising separating the plurality of polypeptides prior to protease digestion according to at least one biological characteristic to obtain subsets of polypeptides.

11. The method of claim 10, wherein the at least one biological characteristic is molecular weight.

12. The method of claim 9, wherein separation is performed by gel electrophoresis and slicing a gel into a plurality of pieces each piece comprising a subset of polypeptides.

13. The method of claim 1, wherein the identifying characteristic is determined by performing multistage mass spectrometry.

14. A method comprising determining the presence, absence or level of one or more phosphorylated peptides identified using the method of claim 1 in a plurality of cells having a cell state and determining the degree of correlation between the presence, absence or level of the phosphorylated polypeptide with the cell state.

15. An isolated peptide of about 5-50 amino acids comprising an amino acid sequence which is a subsequence of a sequence according to any of the proteins listed in Table 4 and which comprise a phosphorylation site within said subsequence.

16. The isolated peptide of claim 15, wherein the peptide comprises an amino acid sequence selected from the group of amino acid sequences shown in Table 4.

17. The isolated peptide of claim 16, wherein the peptide comprises an amino acid sequence selected from the group of amino acid sequences shown in Table 4.

18. An isolated polypeptide selected from a polypeptide listed in Table 4 or a subsequence thereof and which is modified at a modification site as shown in the table.

19. The isolated polypeptide of claim 19 wherein the modification is acetylation or phosphorylation.

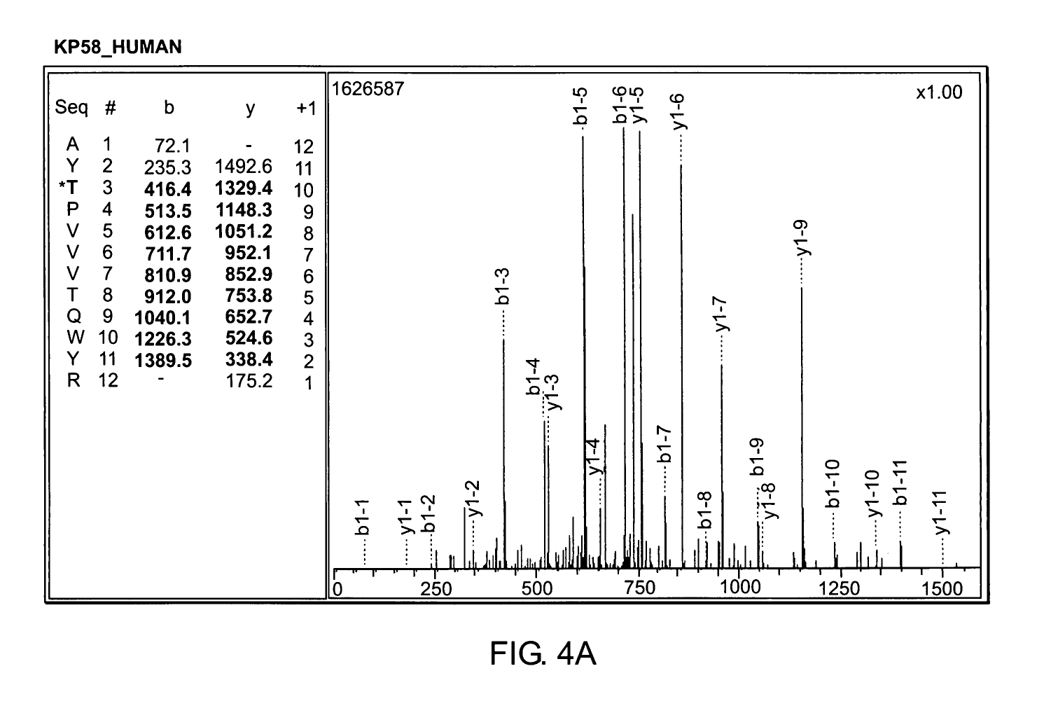

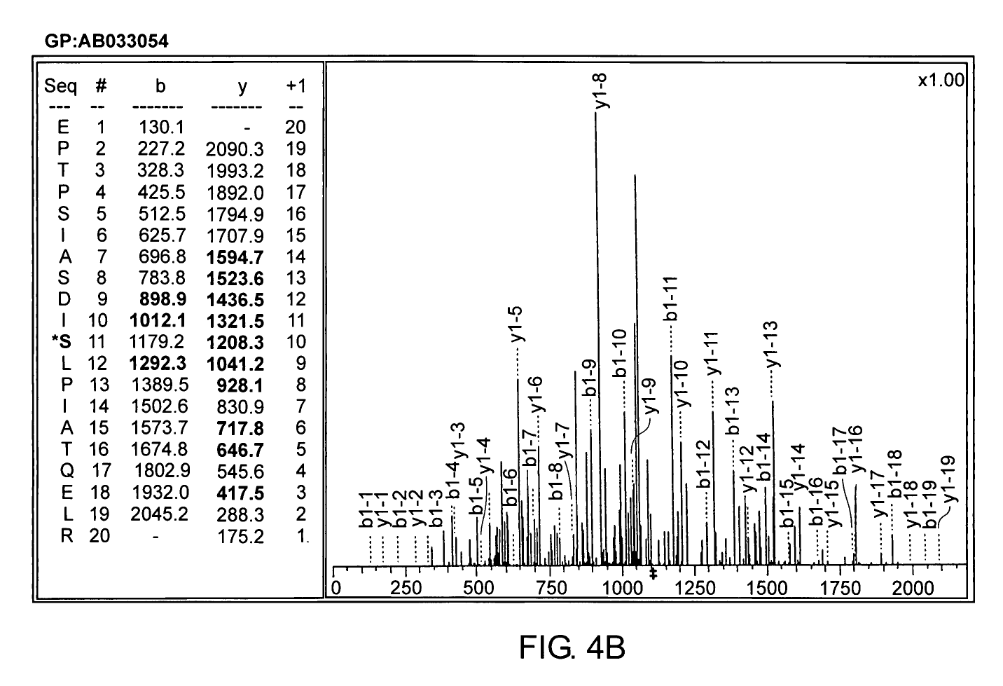

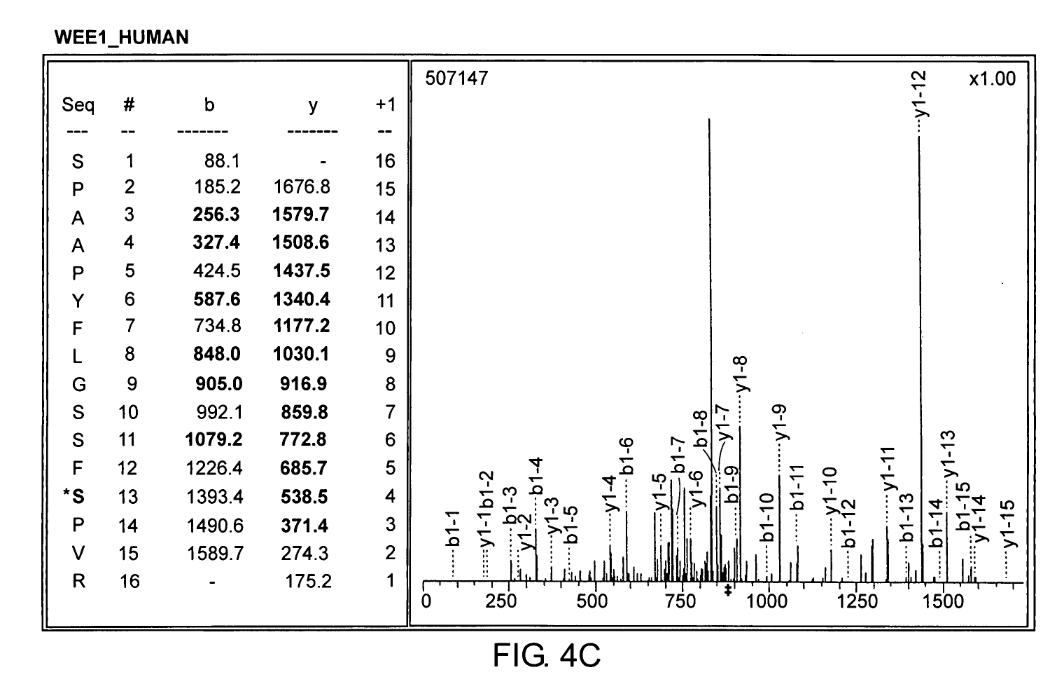

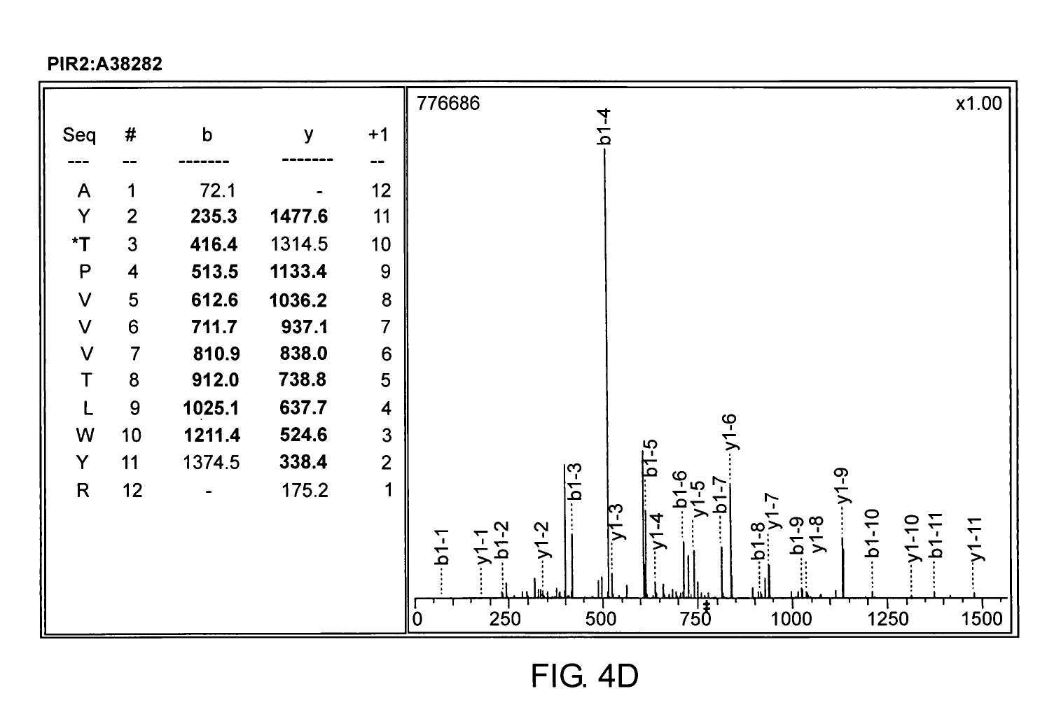

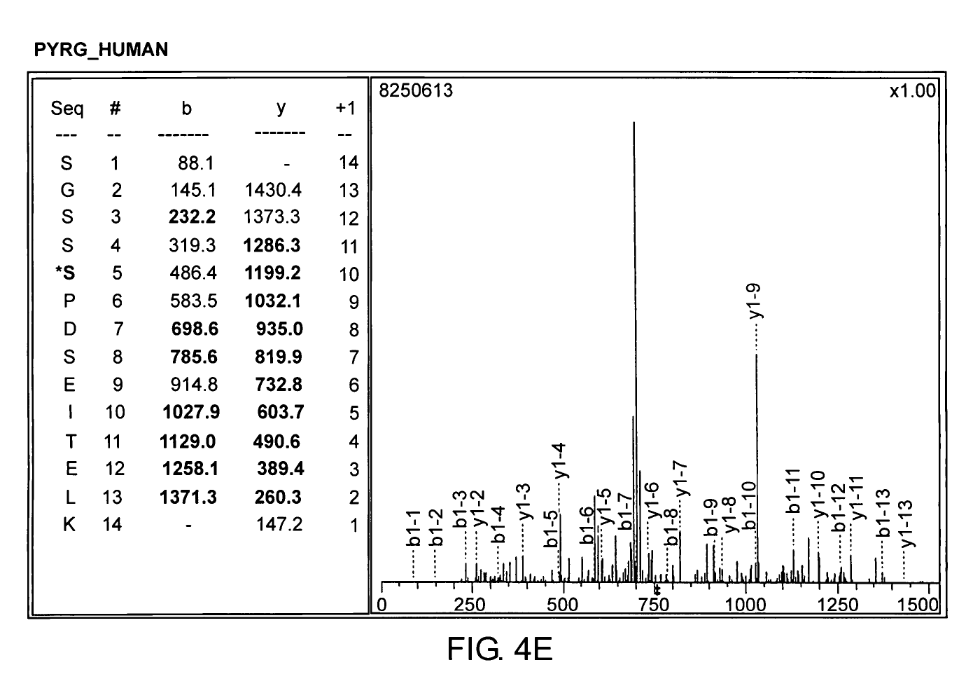

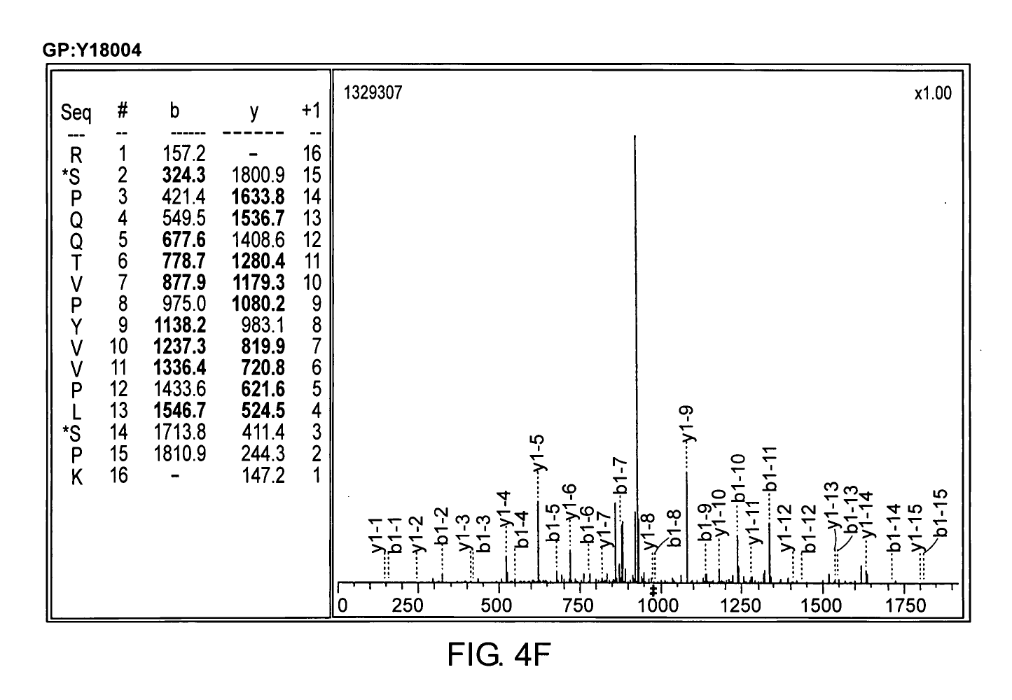

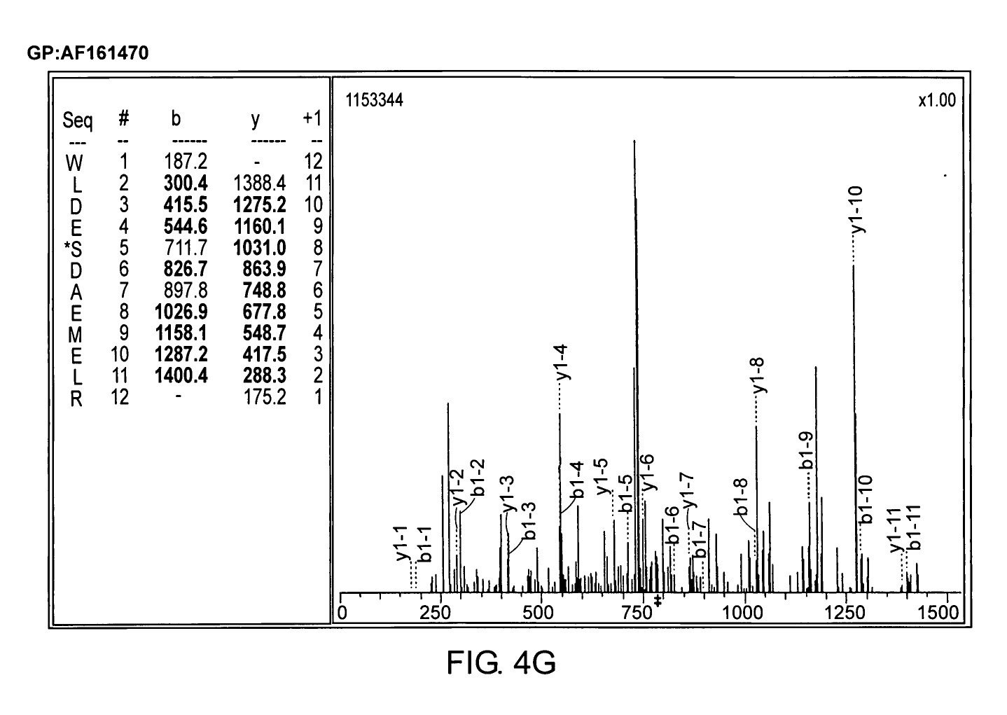

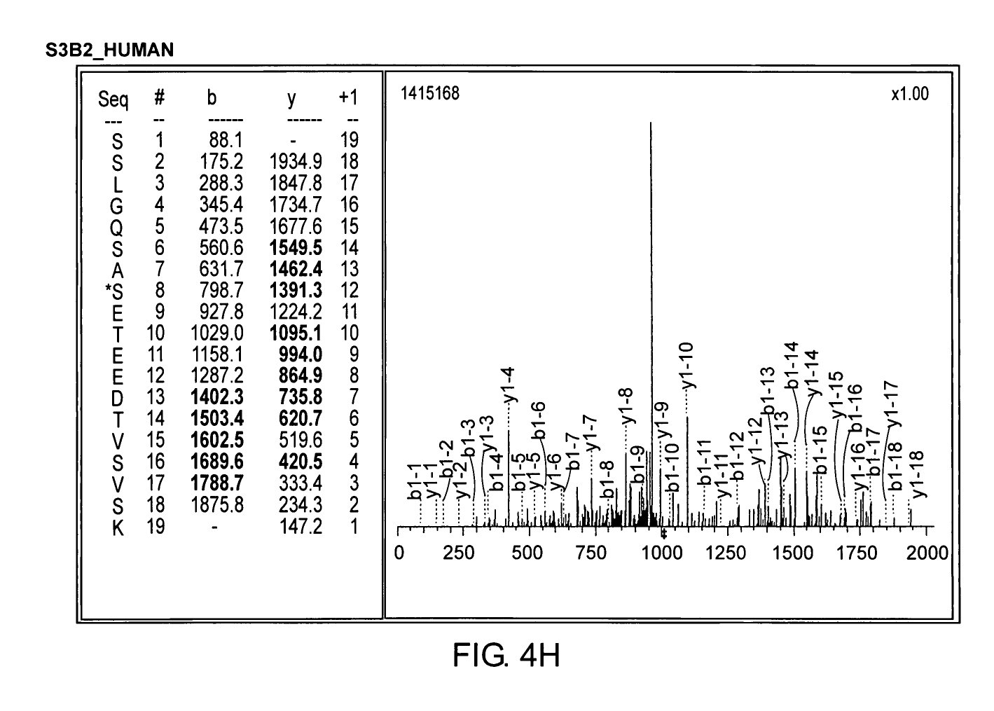

20. An isolated peptide comprising a mass spectral peak signature selected from the group of mass spectral peak signatures as shown in FIGS. 4A-I.

21. An isolated peptide comprising an amino acid sequence selected from the group of sequences shown in FIGS. 4A-I.

22. A method for identifying a treatment that modulates phosphorylation of an amino acid in a target polypeptide, comprising:
subjecting a sample comprising the target polypeptide to a treatment;
determining the level of phosphorylation of one or more amino acids in the target polypeptide before and after treatment;
identifying a treatment that results in a change of the level of modification of the one or more amino acids after treatment;
wherein the level of phosphorlyation is determined by digesting the target polypeptide with a protease and identifying the presence and/or level of a peptide identified according to the method of claim 1.

23. A method for generating a peptide standard comprising labeling a peptide obtained by the method of claim 1 with a mass altering label.

24. A pair of peptide standards comprising a peptide obtained by the method of claim 22, wherein the peptide is phosphorylated and a corresponding peptide comprising an identical amino acid sequence but which is not phosphorylated.

25. The method of claim 22, wherein the treatment comprises exposing the sample to a modulator of kinase activity.

26. The method of claim 22, wherein the treatment comprises exposing the sample to a modulator of phosphatase activity.

27. The method of claim 25, wherein the modulator is an agonist.

28. The method of claim 26, wherein the modulator is an agonist.

29. The method of claim 25, where the modulator is an antagonist.

30. The method of claim 26, where the modulator is an antagonist.

31. A system comprising a computer memory comprising data files storing information relating to the identifying characteristics of positively charged peptides identified in claim 1 and a data analysis module capable of executing instructions for organizing and/or searching the data files.

32. The system according to claim 29, wherein the information comprises the amino acid sequences of phosphorylated and acetylated proteins.

33. The system according to claim 29, wherein the information comprises the sites of phosphorylation of a plurality of polypeptides.

34. The system according to claim 30, wherein the information comprises the sites of phosphorylation of a plurality of polypeptides.

35. The system according to claim 29, wherein the information comprises the sites of phosphorylation of a plurality of polypeptides in a cell having a cell state.

36. The system according to claim 33, wherein the cell is from a patient having a disease.

37. The system according to claim 33, wherein the information comprises the sites of phosphorylation of a plurality of polypeptides in an organelle from a cell having a cell state.

38. The system according to claim 34, wherein the information comprises the sites of phosphorylation of a plurality of polypeptides in an organelle from a cell having a cell state.

39. The method according to claim 1, wherein the sample comprises one or more isolated organelles.

40. The method according to claim 1, wherein the sample comprises one or more isolated nuclei.

41. The method according to claim 1 wherein the plurality comprises at least bout 100,000 different peptides.

42. The method according to claim 1, wherein the identifying characteristic is determined for at least about 10 of the peptides.

43. The method according to claim 1, wherein the identifing characteristic is determined for at least about 100 of the peptides.

44. The method according to claim 1, wherein the identifying characteristic is determined for at least about 1000 of the peptides.

45. A computer program product comprising data relating to the identifying characteristics of positively charged peptides identified in claim 1 and comprising instructions for organizing and/or searching the data.

46. A method for identifying N-terminal peptides in a sample comprising:
providing a biological sample comprising plurality of proteins;
digesting the polypeptides with trypsin, thereby generating a plurality of peptides;
subjecting the peptides to SCX chromatography; and
collecting a fraction of test peptides which are enriched for positively charged peptides having a solution charge state of 1+.
